(19) United States
(12) Patent Application Publication
(10) Pub. No.: US 2024/0342555 A1
(43) Pub. Date: Oct. 17, 2024

(54) SENSING SYSTEMS AND METHODS FOR PROVIDING OPTIMIZED EXERCISE GUIDANCE TO METABOLICALLY UNFIT HOSTS USING CONTINUOUSLY MONITORED ANALYTE DATA

(71) Applicant: Dexcom, Inc., San Diego, CA (US)

(72) Inventors: Matthew L. JOHNSON, San Diego, CA (US); Rush BARTLETT, Austin, TX (US); John PADERI, San Francisco, CA (US); Qi AN, San Diego, CA (US)

(21) Appl. No.: 18/636,210

(22) Filed: Apr. 15, 2024

Related U.S. Application Data
(60) Provisional application No. 63/496,369, filed on Apr. 14, 2023.

(51) Int. Cl. A63B 24/00; A61B 5/00; A61B 5/0205; A61B 5/145; A61B 5/1486

(52) U.S. Cl. CPC A63B 24/0075; A61B 5/0205; A61B 5/14546; A61B 5/14865; A61B 5/4866; A61B 5/7246; A61B 5/7267; A63B 2024/0078

(57) ABSTRACT

Certain aspects of the present disclosure provide a monitoring system comprising a continuous analyte sensor configured to generate a first set of analyte measurements associated with analyte levels of a host, and a sensor electronics module coupled to the continuous analyte sensor and configured to receive and process the first set of analyte measurements.

### CROSS-REFERENCE TO RELATED APPLICATIONS

This application claims priority to and benefit of U.S. Provisional Application No. 63/496,369, filed Apr. 14, 2023, which is hereby assigned to the assignee hereof and hereby expressly incorporated by reference in their entirety as if fully set forth below and for all applicable purposes.

### INTRODUCTION

Exercising and living an active lifestyle is important for managing weight, reducing the risk of disease, improving disease stage, and strengthening bones and muscles. As a person ages, the ability for the body to improve overall fitness and improve or prevent disease is directly correlated with exercise. When a person fails to exercise over long periods of time, there may be negative repercussions such as weight gain, decreased metabolic fitness, decline in liver health and weight gain, to name a few.

Decreased metabolic fitness may be measured by the ability of a person's body (e.g., liver, kidneys and skeletal muscle) to metabolize lactate at rest, during or after an exercise session. The more resilient the liver, muscles and bones of the host, the better the host will be able to process blood lactate at a normal rate at rest and following exercise, which directly contributes to maintenance of a healthy weight and improved liver health and function.

A decline in liver health may lead to common liver diseases such as cirrhosis, non-alcoholic fatty liver disease (NAFLD) or non-alcoholic steatohepatitis (NASH), for example. The American Liver Foundation estimates that more than 20% of the population has NAFLD. It is suggested that a sedentary lifestyle, along with obesity and unhealthy diets may contribute to the high prevalence of NAFLD. When left untreated, NAFLD may progress to NASH, causing serious adverse effects to the body. Once a person develops NASH, the person may experience liver swelling and scarring (i.e., cirrhosis) over time.

Both metabolic fitness and liver health may be tracked using lactate, among other analytes. Lactate is measured and analyzed using various approaches including central laboratory methods, near patient blood gas analysis, and analysis using portable point of care (POC) handheld devices. The central laboratory approach involves transportation of blood samples of a patient to a laboratory via porters or air-tube systems. Unfortunately, the central laboratory approach often suffers from prolonged times between when blood is drawn to when the clinician becomes aware of the test results, resulting in a potential delay in clinical decision-making.

As POC technology has advanced, near-patient, benchtop blood gas analyzers have been made available for lactate testing. However, these devices are not portable and their availability is usually restricted to individual specialized units, e.g., emergency departments (EDs) and intensive care units (ICUs). Further, sample turnaround time of test results may be affected by delays in transportation to the ED or ICU, when the sample was drawn outside these major units.

For this reason, small hand-held devices, much like glucose meters, have been made available for lactate measuring and analysis. A host may carry a self-monitoring lactate monitor which typically requires the host to prick his or her finger to measure his or her lactate levels. However, given the inconvenience associated with traditional finger pricking methods, it is unlikely that a host will take a timely lactate measurement. Consequently, the host's lack of engagement with the device can have devastating results. In particular, a host who forgoes engaging with the device may also fail to monitor their metabolic fitness or liver heath outside of the device's use. Without proper management, the host's metabolic fitness and liver condition may significantly deteriorate, additional health issues may arise, and, in some cases, lead to an increased risk or likelihood of mortality.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example therapy management system used in connection with implementing embodiments of the present disclosure.

FIG. 2 is a diagram conceptually illustrating an example continuous analyte monitoring system including example continuous analyte sensor(s) with sensor electronics, according to certain embodiments of the present disclosure.

FIG. 3 illustrates example inputs and example metrics that are calculated based on the inputs for use by the therapy management system of FIG. 1, according to certain embodiments of the present disclosure.

FIG. 4 is a flow diagram depicting an example method for classifying a host as a healthy host, an athlete, or a metabolically unfit host, according to certain embodiments of the present disclosure.

FIGS. 5A-5C describe an example method for optimizing exercise sessions and providing feedback to a metabolically unfit host, according to certain embodiments of the present disclosure.

FIG. 6 illustrates a flow diagram of an example method for providing therapy management support using a continuous analyte monitoring system configured to continuously measure at least lactate levels, according to certain embodiments of the present disclosure.

FIG. 7 is a flow diagram depicting a method for training machine learning models to optimize exercise for a host, according to certain embodiments of the present disclosure.

FIG. 8 is a block diagram depicting a computing device configured to perform the operations of FIGS. 4-6, according to certain embodiments of the present disclosure.

FIG. 9 is a graph illustrating three lactate signal traces of three different hosts throughout three corresponding exercise sessions with increasing workload, according to certain embodiments of the present disclosure.

FIGS. 10A-10B depict exemplary enzyme domain configurations for a continuous multi-analyte sensor, according to certain embodiments of the present disclosure.

FIGS. 10C-10D depict exemplary enzyme domain configurations for a continuous multi-analyte sensor, according to certain embodiments of the present disclosure.

FIG. 10E depicts an exemplary enzyme domain configuration for a continuous multi-analyte sensor, according to certain embodiments of the present disclosure.

FIGS. 11A-11B depict alternative views of an exemplary dual electrode enzyme domain configuration for a continuous multi-analyte sensor, according to certain embodiments of the present disclosure.

FIGS. 11C-11D depict alternative views of an exemplary dual electrode enzyme domain configuration for a continuous multi-analyte sensor, according to certain embodiments of the present disclosure.

FIG. 11E depicts an exemplary dual electrode configuration for a continuous multi-analyte sensor, according to certain embodiments of the present disclosure.

FIG. 12A depicts an exemplary enzyme domain configuration for a continuous multi-analyte sensor, according to certain embodiments of the present disclosure.

FIGS. 12B-12C depict alternative exemplary enzyme domain configurations for a continuous multi-analyte sensor, according to certain embodiments of the present disclosure.

FIG. 13 depicts an exemplary enzyme domain configuration for a continuous multi-analyte sensor, according to certain embodiments of the present disclosure.

FIGS. 14A-14D depict alternative views of exemplary dual electrode enzyme domain configurations G1-G4 for a continuous multi-analyte sensor, according to certain embodiments of the present disclosure.

FIG. 15A depicts an exemplary lactate sensor, according to certain embodiments of the present disclosure.

FIG. 15B depicts a cross-sectional view of the electroactive section of the example lactate sensor of FIG. 13A, according to certain embodiments of the present disclosure.

FIGS. 16A-16C depict an exemplary embodiment of a continuous analyte sensor system implemented as a wearable lactate sensor, according to certain embodiments of the present disclosure.

FIGS. 17A-17G depict a single sided, co-planar analyte sensor assembly, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Effective exercise routines may be linked to improvement in metabolic fitness and disease state, specifically liver disease. Exercise routines and physical activity may be recommended by a doctor for management of liver disease and overall improvement in metabolic fitness. However, a single exercise type is not effective for all hosts, and often it is not even practical to determine the effectiveness of the type of exercise performed by a host. For example, non-analyte parameters (e.g. heart rate, respiratory rate) are not sufficient to determine host's metabolic response to exercise. Hosts may have a variety of different goals for exercising, whether it be a healthy host with a weight loss goal or a goal of improving athletic performance over time, or a metabolically unfit host with a goal of improving liver function or generally improving metabolic fitness. As a result, certain exercise types may be more effective for specific hosts depending on their physiological/fitness state and goals. However, a host may be unaware of the types of exercise most suited for their corresponding physiological state and/or goal(s), and/or other exercise parameters to more effectively attain the host's goals (e.g., duration, frequency, etc.). Optimizing an exercise session may not only be beneficial to athletes and healthy hosts, but to help improve metabolic fitness and liver function in metabolically unfit hosts and prevent metabolically unfit hosts from encountering dangerous situations such as hyperglycemia and hypoglycemia.

As an example, a metabolically unfit host may benefit from Zone 2 training (“Zone 2”), also referred to as aerobic exercise, to achieve improved metabolic fitness. Zone 2 exercises may include walking, rowing, swimming or light jogging, for example. Generally, Zone 2 is the maximal exercise rate at which lactate generation and clearance are equal before, during, and after an exercise session and may demonstrate the host's metabolic fitness and liver function. As described herein, an exercise session is a period of elevated physiologic activity related to physical activity, which may include a time period during exercise, a time period post-exercise when the host's lactate levels are still elevated, or a time period (e.g., 24-48 hours) post-exercise when the host experiences an increase in metabolic rate. In particular, regular Zone 2 exercise may help improve metabolic fitness by decreasing baseline lactate over time, increasing lactate clearance rate, increasing fat loss, decreasing resting heart rate, lowering blood pressure, improving insulin resistance, improving muscle mitochondrial function, and increasing endurance, for example.

In another example, a healthy host or athlete may benefit from high intensity interval training (HIIT) or anaerobic exercise to promote a long-term increase in energy expenditure. HIIT exercise may include high-intensity plyometric movements, or running sprints followed by low intensity rest periods, for example. HIIT exercises help to improve metabolic fitness by promoting energy expenditure over time, improving oxygen consumption, decreasing resting heart rate, lowering blood pressure, and improving resting metabolic rate.

In another example, a metabolically unfit host may benefit from resistance training or an activity that involves exercising a muscle or muscle group against external resistance (e.g., weights or a resistance band, etc.) to increase muscle mass or increase long term energy expenditure. Similar to HIIT exercise, resistance training results in an increase in long term fat oxidation and energy expenditure as the body expends energy repairing muscles in the days following a resistance training session. Therefore, metabolically unfit hosts experiencing decreasing muscle mass (e.g., sarcopenia) or hosts with a goal of improving muscle mass and/or strength may benefit from a resistance training exercise session.

In yet another example, a metabolically unfit host may benefit from HIIT exercise to increase fat burning over time following the HIIT exercise. In addition, as described above, HIIT exercise may help improve metabolic fitness, improve oxygen consumption, and improve resting metabolic rate, even in the time period following the exercise session, typically up to one to two days post-exercise. In certain embodiments, the metabolically unfit host may be instructed to avoid consuming foods containing glucose or fructose to increase the benefit of the HIIT exercise session.

As discussed herein, regular physical activity, specifically for metabolically unfit hosts (e.g. hosts with metabolic disorders or liver disease), is crucial for helping such hosts with managing their disease. However, management of liver disease and metabolic fitness presents many challenges for hosts and clinicians, as a confluence of various factors can impact a host's metabolic fitness and ability to exercise, thus affecting the accuracy of exercise guidance provided.

Accordingly, embodiments described herein provide systems and methods for providing optimized exercise predictions and/or recommendations to increase an overall health of a host. The optimized exercise predictions and/or recommendations are accomplished by classifying hosts based on their physiological state and goals, and for optimizing exercise sessions for hosts based on such classifications. Optimizing exercise sessions may include providing individualized exercise recommendations and/or automatically controlling exercise machines (e.g., by sending data including instructions to such exercise machines) to achieve a certain set of optimized exercise parameters and/or providing real-time guidance to a host to achieve such parameters in an effort to improve metabolic fitness and an overall health of the host based on analyte and/or non-analyte parameters measured for the host. Further, the embodiments described herein also enable measuring and providing feedback relating to improvement or deterioration of the hosts' disease state over time. These optimized exercise predictions and/or recommendations may be followed by the host, resulting in a improvement of various aspects of the host's physiological state, including the host's blood lactate and/or glucose levels. Optimized exercise predictions and/or recommendations may also be determined in real-time.

In certain embodiments, over time the system may identify the results of earlier predictions and/or recommendations and may continually refine future predictions and/or recommendations based at least in part on earlier predictions and/or recommendations. The refinement of predictions and/or recommendations may be performed in real-time. The refinement over time may further improve the accuracy of such predictions and/or recommendations, which may in turn further improve the health of hosts who adjust their diet, exercise, and other aspects of their life in response to such predictions and/or recommendations.

Further, by maximizing the accuracy of an initial classification of a host, based on a host profile, host input or a trial exercise session, and subsequently correlating this classification to one or more optimized exercise predictions and/or recommendations for the host, future adjustments to such recommendations may be minimized. The decrease in future adjustments may in turn minimize the computation and/or network load requirements for hardware computing devices tasked with determining and presenting such recommendations. In view of a large corpus of hosts all requesting predictions and/or recommendations, this will significantly reduce network and/or computation requirements of associated hardware systems, thereby improving a performance of such systems. It should also be noted that manually monitoring the analyte and/or non-analyte data of a host and adjusting exercise predictions and/or recommendations in real-time based on such manual monitoring is not feasible given the high frequency of analyte sampling, the large quantity of physiological and goal data involved, and the complexity of calculations being performed.

In addition, as described above, existing methods of measuring lactate may, at least in certain cases, not enable or be conducive to optimizing exercise sessions for hosts in real-time. For example, manually monitoring the physiological state of a host (including lactate levels of the host) and adjusting exercise predictions and/or recommendations in real-time based on such manual monitoring is not feasible given the high frequency of analyte sampling, the large quantity of physiological and goal data involved, and the complexity of calculations being performed. As such, the systems described herein include a continuous analyte monitoring system configured to measure lactate levels in a continuous manner, thereby, providing real-time lactate measurements that can be used to optimize exercise sessions for hosts in real-time. Note that, as used herein, real-time may also include near real-time measurements and/or optimization recommendations (e.g., to account for a biological delay in lactate levels reaching the blood and/or interstitial fluid). Further, optimization of exercise sessions and feedback related to exercise sessions may be provided retrospectively, as described further herein.

As used herein, the term “continuous” analyte monitoring refers to monitoring one or more analytes in a fully continuous, semi-continuous, periodic manner, which results in a data stream of analyte values over time. A data stream of analyte values over time is what allows for meaningful data and insight to be derived using the algorithms described herein for classifying a host, optimizing exercise sessions, and providing feedback related to exercise sessions. In other words, single point-in-time measurements collected as a result of a patient visiting their health care professional every few months results in sporadic data points (e.g., that are, at best, months apart in timing) that cannot form the basis of any meaningful data or insight to be derived. As such, without the continuous analyte monitoring system of the embodiments herein, it is simply impossible to continuously classify a host and optimize one or more exercise sessions over time, as well as continuously provide feedback related to exercise sessions, as described herein.

Further, the data stream of analyte values collected over time, with the continuous analyte monitoring system presented herein, include real-time analyte values, which allows for deriving meaningful data and insight in real-time using the systems and algorithms described herein. The derived real-time data and insight in turn allows for providing real-time classification of a host and exercise optimization feedback, as well as real-time feedback related to exercise sessions. Real time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) calibrating the count values to generate at least lactate and/or other analyte concentration values using calibration techniques described herein to account for the sensitivity of the continuous analyte sensor(s), and (3) transmitting measured lactate and/or other analyte concentration data, including lactate and/or other analyte concentration values, to a display device via wireless connection.

For example, the at least one sensor electronics module may be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured lactate and/or other analyte concentration data to a display device at a particular transmission period (or rate), which may be the same as (or longer than) the sampling period, such as every 1 minute (0.016 Hz), 5 minutes, 10 minutes, etc.

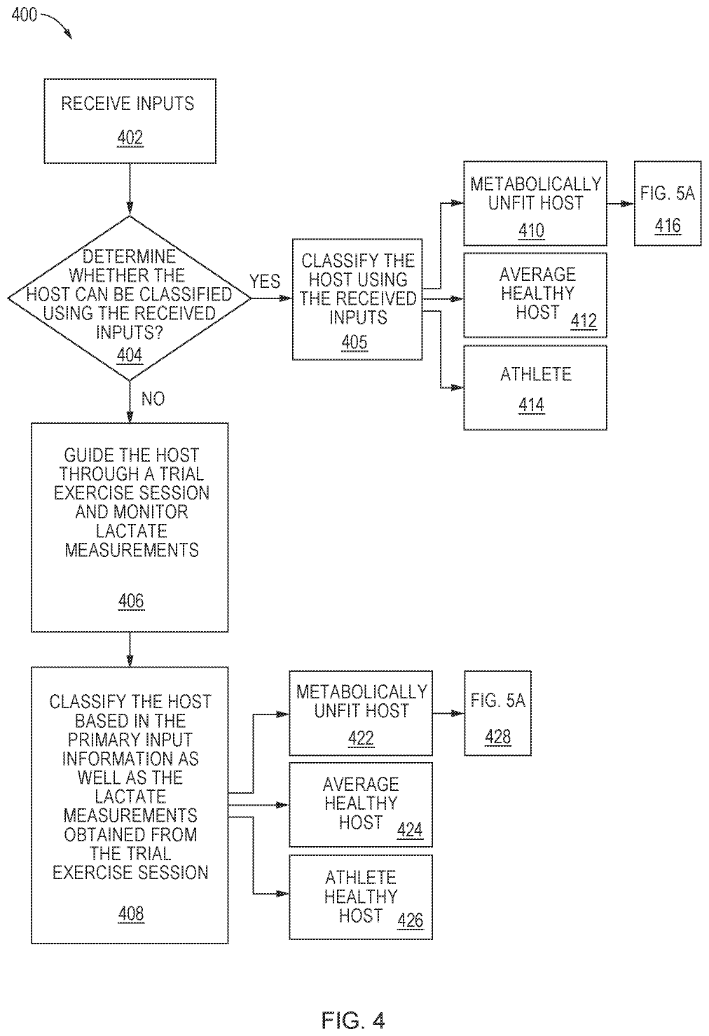

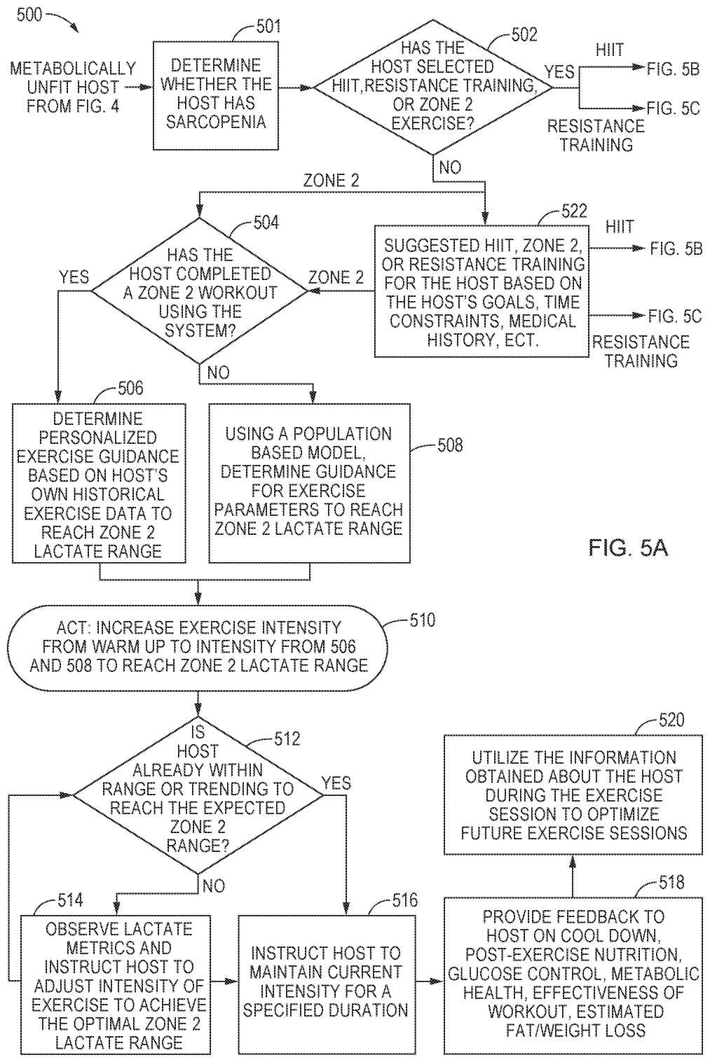

The real-time analyte data that is continuously generated by the continuous analyte monitoring system described herein, therefore, allows the therapy management system herein to classify a host and optimize an exercise session, as well as provide feedback on an exercise session, in real-time, which is technically impossible to perform using existing or conventional techniques or systems. Further, because of the real-time nature of this data, it is also humanly impossible to continuously process a real-time data stream of analyte values over time to derive meaningful data and insight using the algorithms and systems described herein to classify a host and optimize an exercise session, as well as provide feedback on an exercise session. In other words, deriving meaningful data and insight from a stream of real-time data that is continuously generated, processed, calibrated, and analyzed, using the algorithms and systems described herein, is not a task that can be mentally performed. For example, executing the algorithms described in relation to FIGS. 4-7 in real-time and on a continuous basis, which would involve using a stream of real-time data that is continuously generated by a host's continuous analyte monitoring system and/or significantly large amount of population data (e.g., hundreds or thousands of data points for each one of thousands or millions of hosts in the host population) is not a task that can be mentally performed, especially in real-time at times.

Further, certain embodiments herein are directed to a technical solution to a technical problem associated with analyte sensor systems. In particular, each analyte sensor system that is manufactured by a sensor manufacturer might perform slightly different. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current may be expressed in units of picoAmps (pA) or counts. The amount of measured analyte may be expressed as a concentration level in units of milligrams per deciliter (mg/dL), and the calibration sensitivity may be expressed in units of pA/(mg/dL) or counts/(mg/dL). The calibration baseline refers to the amount of electrical current produced by the analyte sensor when no analyte is detected, and may be expressed in units of pA or counts.

The calibration sensitivity, calibration baseline, and other information related to the sensitivity profile for the analyte sensor system may be programmed into the sensor electronics module of the analyte sensor system during the manufacturing process, and then used to convert the analyte sensor electrical signals into measured analyte concentration levels. For example, the calibration slope (calibration sensitivity) may be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels may be determined using a sensitivity function M (t) that is based on the initial in vivo sensitivity (M₀) and the final in vivo sensitivity (M_{f}). The sensitivity function M(t) may expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$A C L = count / M ( {t}_{i} ) \qquad Eq . 1$$

A calibration baseline (baseline) may also be used to determine a measured analyte concentration level (ACL) from an analyte sensor count value (count) at a time tᵢ, and Equation 2 presents one technique:

$$A C L = ( count - baseline ) / M ( {t}_{i} ) \qquad Eq . 2$$

### Example Therapy Management System Including an Example Continuous Analyte Sensor for Predicting Exercise Parameters for Exercise Optimization

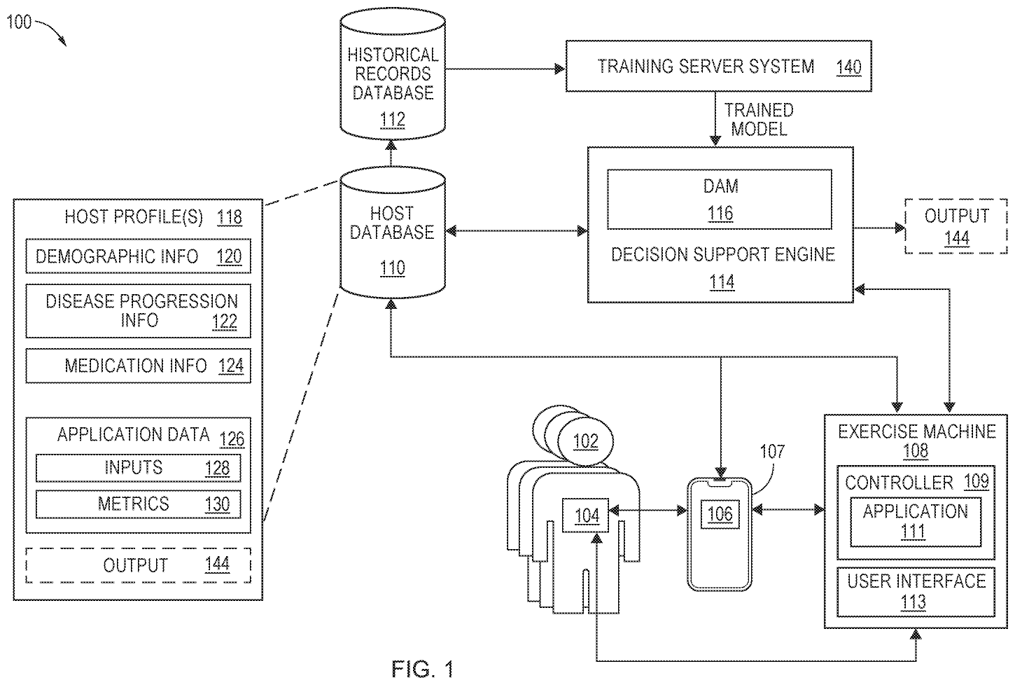

FIG. 1 illustrates an example therapy management system 100 for optimizing exercise parameters to improve metabolic fitness of metabolically unfit hosts 102 (individually referred to herein as a host and collectively referred to herein as hosts), using a continuous analyte monitoring system 104 configured to continuously measure at least lactate levels. A host, in certain embodiments, may be a metabolically unfit host, who may suffer from liver issues (e.g., disease, condition, or failure), a host with a metabolic disorder, or any other conditions impacting the metabolic fitness of the host. As discussed herein, metabolic fitness refers to a host's ability to clear lactate generally, which, in some cases, could be impacted by the host's liver issues (function, disease, failure, etc.) or other metabolic disorders.

Lactate is the conjugate base of lactic acid. Lactate is produced from pyruvate (e.g., glucose is broken down to pyruvate) through enzyme lactate dehydrogenase during normal metabolism and exercise. Approximately up to 70% of lactate is metabolized by the liver. However, in very early liver disease, such as NAFLD, lactate metabolism is altered, which may lead to elevated levels of lactate in the body. Further, as the liver disease progresses, lactate fasting level increases, lactate metabolism becomes impaired, and lactate half-life increases. Lactate elevation may be caused by such increased production, decreased clearance, or both in combination. Accordingly, lactate may need to be continuously monitored to continually assess parameters such as lactate clearance rate (also indicative of lactate half-life), lactate levels, lactate production rates, and lactate baselines for determining metabolic fitness and liver health in real-time.

In certain embodiments, therapy management system 100 includes continuous analyte monitoring system 104, a display device 107 that executes application 106, a therapy management engine 114, a host database 110, a historical records database 112, a training server system 140, and a therapy management engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, endogenous insulin, acarboxyprothrombin; acylcarnitine; endogenous insulin; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones (e.g., insulin) naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include lactate, glucose, and/or ketones, in some cases other analytes listed above may also be considered.

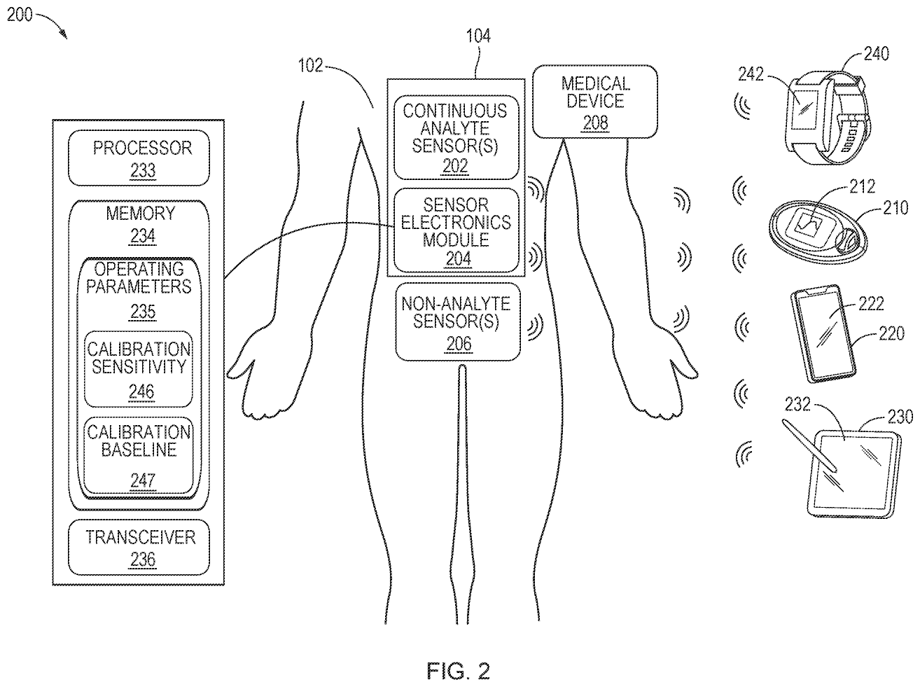

In certain embodiments, continuous analyte monitoring system 104 is configured to continuously measure one or more analytes and transmit the analyte measurements to display device 107 for use by application 106. In certain embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device such as a laptop computer, a smart watch, a fitness tracker, a cycling computer, a tablet, or any other computing device capable of executing application 106. In certain embodiments, continuous analyte monitoring system 104 may further be configured to directly transmit analyte measurements to exercise machine 108 through a wireless connection (e.g., Bluetooth connection). In certain other embodiments, exercise machine 108 may receive analyte measurements provided by continuous analyte monitoring system 104 through display device 107. Continuous analyte monitoring system 104 may be described in more detail with respect to FIG. 2.

Exercise machine 108 may be a treadmill, an elliptical, a stationary bike, a StairMaster, an indoor rower, a smart cable machine, or any other type of exercise machine that can execute a software application.

Application 106 is a mobile health application that is configured to receive and analyze analyte measurements from analyte monitoring system 104. For example, application 106 stores information about a host, including the host's analyte measurements, in a host profile 118 of the host for processing and analysis as well as for use by the therapy management engine 114 to provide therapy management support recommendations or guidance to the host. Application 111 is a software application executing on exercise machine 108 and refers to a set of instructions for, at least in part, controlling the operations of exercise machine 108. Application 111 may perform some or all of the functionality of application 106. For example, application 111 may be configured to receive and analyze analyte measurements from analyte monitoring system 104, store information about a host, including the host's analyte measurements, in a host profile 118 of the host, and/or perform some or all of the operations of therapy management engine 114.

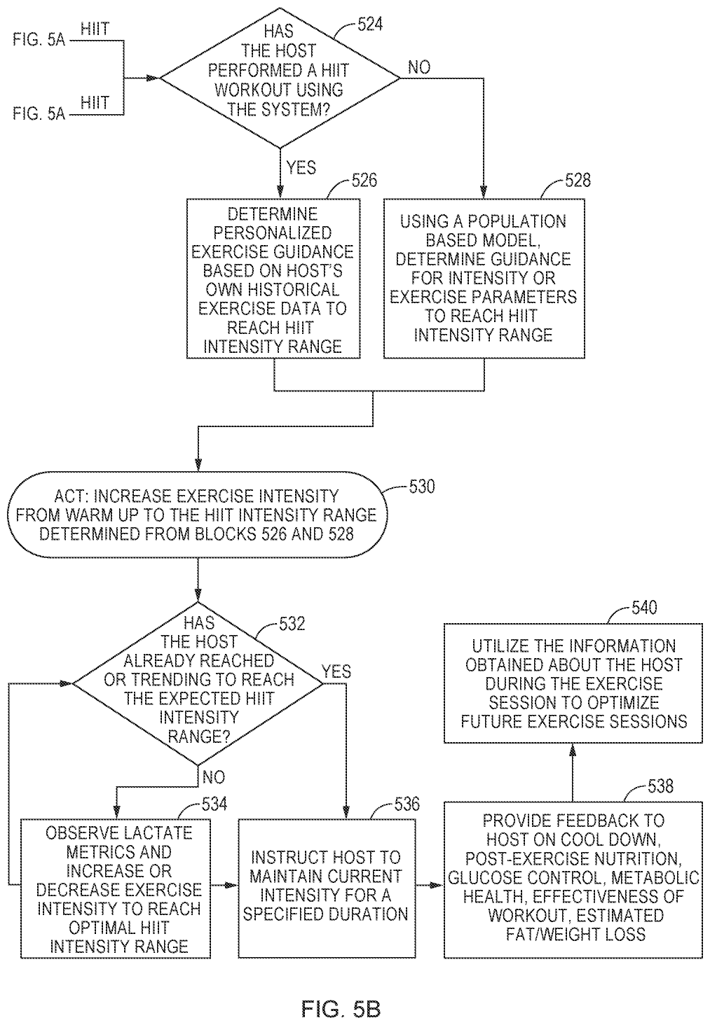

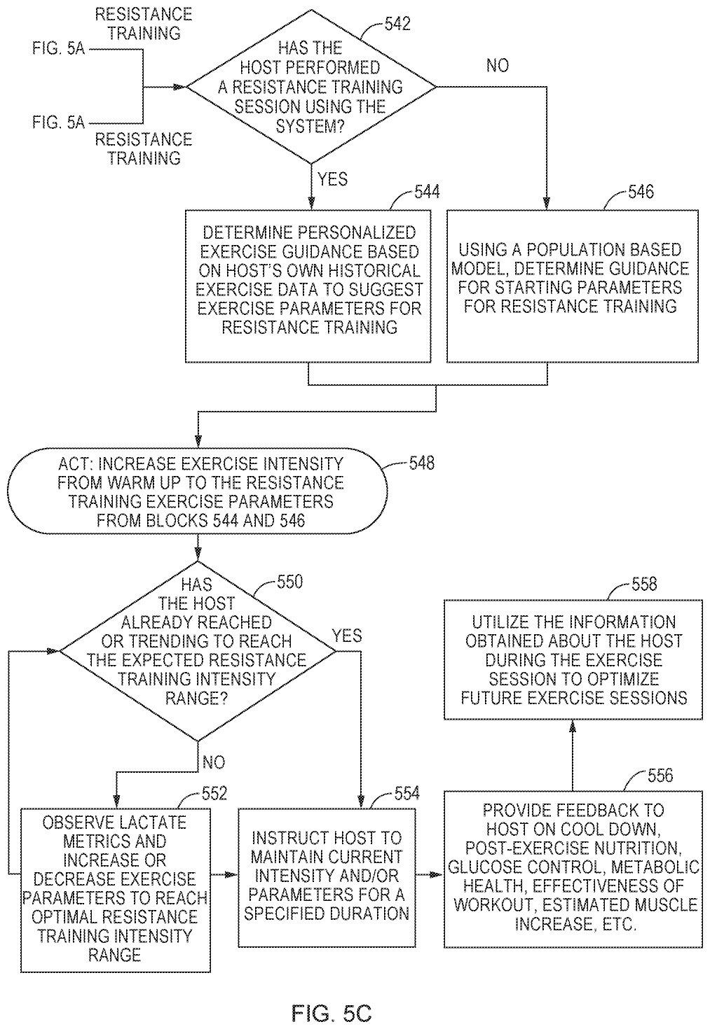

Note that, any reference to therapy management engine 114 providing a suggestion, instruction, or recommendation to the host can alternatively be automatically provided to the application 111 of exercise machine 108 to automatically implement the suggestion, instruction, or recommendation. The output of any step described in reference to FIGS. 5A-7 may be automatically sent to the application 111 of exercise machine 108 to adjust exercise parameters (e.g., speed, incline, resistance, repetitions, weight, etc.) automatically according to the corresponding suggestion, instruction or recommendation. For example, as described herein, therapy management engine 114 may instruct a host to increase intensity (e.g., speed, incline, etc.) in order to reach a desired lactate threshold. In this example, instead of instructing the host, therapy management engine 114 may communicate directly with application 111 to automatically adjust exercise parameters of exercise machine 108 to the desired increased intensity.

Therapy management engine 114 refers to a set of software instructions with one or more software modules, including data analysis module (DAM) 116. In certain embodiments, therapy management engine 114 executes entirely on one or more computing devices in a private or a public cloud. In such embodiments, application 106 and/or application 111 communicate with therapy management engine 114 over a network (e.g., Internet). In certain other embodiments, therapy management engine 114 executes partially on one or more local devices, such as display device 107 and/or exercise machine 108, and partially on one or more computing devices in a private or a public cloud. In certain other embodiments, therapy management engine 114 executes entirely on one or more local devices, such as display device 107 and/or exercise machine 108. As discussed in more detail herein, therapy management engine 114, may provide therapy management support recommendations to the host via application 106 and/or application 111. Therapy management engine 114 provides therapy management support recommendations based on information included in host profile 118.

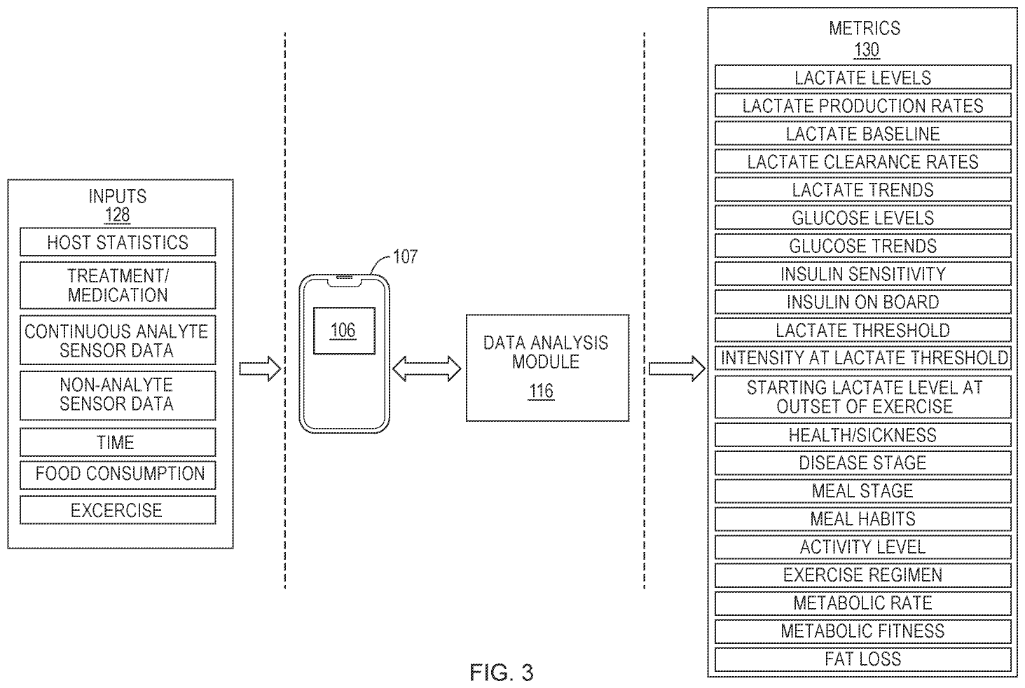

Host profile 118 may include information collected about the host from application 106. For example, application 106 provides a set of inputs 128, including the analyte measurements associated with one or more analytes received from continuous analyte monitoring system 104 that are stored in host profile 118. In certain embodiments, inputs 128 provided by application 106 include other data in addition to analyte measurements. For example, application 106 may obtain additional inputs 128 through manual host input, one or more other non-analyte sensors or devices, other applications executing on display device 107, etc. Non-analyte sensors and devices include one or more of, but are not limited to, an insulin pump, respiratory sensor, sensors or devices provided by display device 107 (e.g., accelerometer, camera, global positioning system (GPS), heart rate monitor, electrocardiogram (ECG), etc.) or other host accessories (e.g., a smart watch, a continuous positive airway pressure (CPAP) machine, or a fitness tracker), or any other sensors or devices that provide relevant information about the host (e.g., sensors on exercise equipment). Inputs 128 of host profile 118 provided by application 106 are described in further detail below with respect to FIG. 3.

DAM 116 of therapy management engine 114 is configured to process the set of inputs 128 to determine one or more metrics 130. Metrics 130, discussed in more detail below with respect to FIG. 3, may, at least in some cases, be generally indicative of the health or state of a host, such as one or more of the physiological state of a host, trends associated with the health or state of a host, etc. In certain embodiments, metrics 130 may then be used by therapy management engine 114 as input for providing guidance to a host. As shown, metrics 130 are also stored in host profile 118.

Host profile 118 also includes demographic info 120, disease info 122, and/or medication info 124. In certain embodiments, such information may be provided through host input or obtained from certain data stores (e.g., electronic medical records, etc.). In certain embodiments, demographic info 120 may include one or more of the host's age, BMI (body mass index), ethnicity, gender, etc. In certain embodiments, disease info 122 may include information about one or more diseases of a host, including relevant information pertaining to the host's metabolic fitness, liver disease, diabetes, kidney disease, and/or any conditions or diseases relevant to metabolic fitness. In certain embodiments, disease info 122 may also include the length of time since diagnosis, the level of disease control, level of compliance with disease management therapy, other types of diagnoses (e.g., heart disease, obesity), etc. In certain embodiments, disease info 122 may include hospitalizations and/or surgical history. In certain embodiments, disease info 122 may include other measures of health (e.g., heart rate, stress, sleep, etc.) or fitness (e.g., cardiovascular endurance, metabolic state, gait information, muscular strength and/or power, muscular endurance, and other measures of fitness), and/or the like.

In certain embodiments, medication info 124 may include information about the amount and type of a medication taken by a host.

In certain embodiments, medication information may include information about the consumption of one or more drugs known to damage the liver (e.g., affect lactic clearance) and/or lead to liver toxicity. One or more drugs known to damage the liver and/or lead to liver toxicity may include antibiotics such as amoxicillin/clavulanate, clindamycin, erythromycin, nitrofurantoin, rifampin, sulfonamides, tetracyclines, trimethoprim/sulfamethoxazole, and drugs used to treat tuberculosis (isoniazid and pyrazinamide), anticonvulsants such as tarbamazepine, thenobarbital, phenytoin, and valproate, antidepressants such as bupropion, fluoxetine, mirtazapine, paroxetine, sertraline, trazodone, and tricyclic antidepressants such as amitriptyline, antifungal drugs such as ketoconazole and terbinafine, antihypertensive drugs (e.g., drugs used to treat high blood pressure or sometimes kidney or heart disorder) such as captopril, enalapril, irbesartan, lisinopril, losartan, and verapamil, antipsychotic drugs such as phenothiazines (e.g., such as chlorpromazine) and risperidone, heart drugs such as amiodarone and clopidogrel, hormone regulation drugs such as anabolic steroids, birth control pills (oral contraceptives), and estrogens, pain relievers such as acetaminophen and nonsteroidal anti-inflammatory drugs (NSAIDs), and other drugs such as acarbose (e.g., used to treat diabetes), allopurinol (e.g., used to treat gout), antiretroviral therapy (ART) drugs (e.g., used to treat human immunodeficiency virus (HIV) infection), baclofen (e.g., a muscle relaxant), cyproheptadine (e.g., an antihistamine), azathioprine (e.g., used to prevent rejection of an organ transplant), methotrexate (e.g., used to treat cancer), omeprazole (e.g., used to treat gastroesophageal reflux), PD-1/PD-L1 inhibitors (e.g., anticancer drugs), statins (e.g., used to treat high cholesterol levels), and many types of chemotherapy, including immune checkpoint inhibitors.

In certain embodiments, medication information may include information about consumption of one or more drugs known to improve liver function. One or more drugs known to improve liver function may include ademetionine, avatrombopag, dehydroemetine, entecavir, glecaprevir and pibrentasvir, lamivudine, metadoxine, methionine, sofosbuvir, velpatasvir, and voxilaprevir, telbivudine, tenofovir, trientine, ursodeoxycholic acid, and the like.

In certain embodiments, host profile 118 is dynamic because at least part of the information that is stored in host profile 118 may be revised or updated over time and/or new information may be added to host profile 118 by therapy management engine 114 and/or application 106. Accordingly, information in host profile 118 stored in host database 110 provides an up-to-date repository of information related to the host.

Host database 110, in certain embodiments, refers to a storage server that operates, for example, in a public or private cloud. Host database 110 may be implemented as any type of datastore, such as relational databases, non-relational databases, key-value datastores, file systems including hierarchical file systems, and the like. In some exemplary implementations, host database 110 is distributed. For example, host database 110 may comprise a plurality of persistent storage devices, which are distributed. Furthermore, host database 110 may be replicated so that the storage devices are geographically dispersed.

Host database 110 includes host profiles 118 associated with a plurality of hosts, including hosts who similarly interact or have interacted in the past with application 106 on their own devices. Host profiles stored in host database 110 are accessible to not only application 106, but therapy management engine 114, and/or exercise machine 108 as well. Host profiles in host database 110 may be accessible to application 106, therapy management engine 114, and/or exercise machine 108 over one or more networks (not shown), such as one or more wireless networks. As described above, therapy management engine 114, and more specifically data analysis module (DAM) 116 of therapy management engine 114, can fetch inputs 128 from a host's profile 118 stored in host database 110 and compute one or more metrics 130 which can then be stored as application data 126 in the host's profile 118.

In certain embodiments, host profiles 118 stored in host database 110 may also be stored in historical records database 112. Host profiles 118 stored in historical records database 112 may provide a repository of up-to-date information and historical information for each host of application 106. Thus, historical records database 112 essentially provides all data related to each host of application 106, where data is stored using timestamps. The timestamp associated with any piece of information stored in historical records database 112 may identify, for example, when the piece of information was obtained and/or updated.

Further, in certain embodiments, historical records database 112 may include data for one or more hosts who are not hosts of continuous analyte monitoring system 104 and/or application 106. For example, analyte data for a host who has used continuous analyte monitoring system 104 and application 106 for a period of five years to optimize exercise sessions and improve the host's metabolic fitness, or a liver condition may have time series analyte data associated with the host maintained over the five-year period.

Further, in certain embodiments, historical records database 112 may include data for one or more hosts who are not hosts of continuous analyte monitoring system 104 and/or application 106. For example, historical records database 112 may include information (e.g., host profile(s)) related to one or more hosts known to be metabolically unfit, as well as information (e.g., host profile(s)) related to one or more hosts previously diagnosed with liver dysfunction. Data stored in historical records database 112 may be referred to herein as population data, which could include hundreds or thousands of data points for each one of thousands or millions of hosts in the host population. In other words, data stored in historical records database 112 and used in certain embodiments described herein could include gigabytes, terabytes, petabytes, exabytes, etc. of data.

Data related to each host stored in historical records database 112 may provide time series data collected over the lifetime of the host. For example, the data may include information about the exercise sessions of the host, including exercise parameters (e.g., speed, incline, wattage) that assisted the host in reaching a desired exercise type. The data may also include physiological information (e.g., height and weight), as well as non-analyte sensor data (e.g., heart rate, respiratory rate, etc.). Such data may indicate a type of exercise the host completed (e.g., HIIT, resistance training, or Zone 2), physiological states of the host, lactate levels of the host, glucose levels of the host, insulin levels of host, free fatty acid levels of the host, states/conditions of one or more organs of the host, habits of the host (e.g., activity levels, food consumption, etc.), medication prescribed throughout the lifetime of the disease, as well as progress of outcomes such as weight loss and metabolic fitness over time, etc.

Although depicted as separate databases for conceptual clarity, in certain embodiments, host database 110 and historical records database 112 may operate as a single database. That is, historical and current data related to hosts of continuous analyte monitoring system 104 and application 106, as well as historical data related to hosts that were not previously hosts of continuous analyte monitoring system 104 and application 106 and/or application 111, may be stored in a single database. The single database may be a storage server that operates in a public or private cloud.

FIG. 1 also illustrates exercise machine 108 including a controller 109 configured to automatically control exercise intensity by adjusting by various exercise parameters, in response to a trigger. For example, the trigger may be host input provided to a software application (“application”) 111 through, for instance, a user interface 113 of the exercise machine 108. In such an example, in response to the host input, the application 111 causes controller 109 to adjust the exercise intensity by adjusting one or more parameters, such as speed, incline, resistance, and other parameters known to one of ordinary skill in the art. The controller may adjust such exercise parameters by sending commands to various electromechanical components (e.g., actuators, motors, etc.) in the exercise machine 108, as known to one of ordinary skill in the art. Application 111 refers to a set of software instructions configured to cause the controller 109 to control the operations of exercise machine 108, among other things as described above.

In another example, the trigger may correspond to exercise intensity instructions received from a person who trains the host (e.g., a personal trainer). The personal trainer may pre-prescribe exercise sessions by type (Zone 2, resistance training, or HIIT), and application 111 of exercise machine 108 may receive instructions corresponding to the pre-prescribed exercise session. Then, controller 109 may control and maintain exercise intensity automatically according to the prescribed exercise type in order to achieve the corresponding exercise zones (Zone 2, resistance training, or HIIT).

In another example, the trigger may correspond to exercise intensity instructions received by application 111 from another device, such as display device 107, to direct the controller 109 to adjust exercise intensity. For example, by executing application 106, display device 107 may generate exercise intensity instructions that may be sent over a wired or wireless connection to exercise machine 108 to control the operations of exercise machine 108. In yet another example, application 111 may be configured to automatically (e.g., without host input and/or instructions from an external device) generate an optimized exercise intensity, in accordance with the embodiments described herein. For example, application 111 may include instructions that, partly or entirely, perform the functionality of therapy management engine 114 for generating optimized exercise intensity instructions, including exercise intensity parameters, that can be used by controller 109 to adjust exercise intensity of the host's exercise on exercise machine 108

Exercise machine 108 may be any kind of resistance machines or endless-path machines. Examples of exercise machine 108 may include a treadmill, elliptical machine, glider machine, climbing machine, rowing machine, stationary bicycle, etc.

As mentioned previously, therapy management system 100 is configured to provide exercise optimization for metabolically unfit hosts using continuous analyte monitoring system 104, including, at least, a continuous lactate sensor. In certain embodiments, to enable such optimization, therapy management engine 114 is configured to (1) provide real-time and or non-real-time exercise therapy management support (e.g., guidance) to the host and or others, including but not limited, to healthcare providers, family members of the host, caregivers of the host, etc., and/or (2) provide real-time instructions to an exercise machine (e.g., exercise machine 108) for automatically adjusting exercise intensity, including various exercise parameters, for the host exercising with the exercise machine. Therapy management support may be intended to provide optimal exercise guidance to provide metabolic fitness improvement and/or optimization and prevent disease development and/or progression (e.g., liver disease or kidney disease).

In particular, therapy management engine 114 may be used to collect information associated with a host in host profile 118 stored in host database 110, to perform analytics thereon to classify the host as a healthy host, an athlete, or a metabolically unfit host. Based on the classification, the therapy management engine 114 may then optimize exercise for the host to help the host achieve their goals. For example, if a host is classified as metabolically unfit, therapy management engine 114 may optimize exercise for the host to improve their metabolic fitness. Optimizing exercise, as described above, may include providing optimized exercise guidance to the host and/or automatically controlling the operations of an exercise machine, such as by controlling speed, incline, resistance, etc. of the machine as well as the duration of the exercise. Host profile 118 may be accessible to therapy management engine 114 over one or more networks (not shown) for performing such analytics.

In certain embodiments, therapy management engine 114 may utilize one or more trained machine learning models capable of performing analytics on information that therapy management engine 114 has collected/received from host profile 118. In the illustrated embodiment of FIG. 1, therapy management engine 114 may utilize trained machine learning model(s) provided by a training server system 140. Although depicted as a separate server for conceptual clarity, in certain embodiments, training server system 140 and therapy management engine 114 may operate as a single server or system. That is, the model may be trained and used by a single server, or may be trained by one or more servers and deployed for use on one or more other servers or systems. In certain embodiments, the model may be trained on one or many virtual machines (VMs) running, at least partially, on one or many physical services in relational and or non-relational database formats.

Training server system 140 is configured to train the machine learning model(s) using training data, which may include data (e.g., from host profiles) associated one or more hosts (e.g., hosts or non-hosts of continuous analyte monitoring system 104 and/or application 106) suffering from a metabolic disorder, previously diagnosed with varying stages of liver disease as well as hosts not previously diagnosed with liver disease or a metabolic disorder (e.g., healthy hosts, athletes, etc.). The training data may be stored in historical records database 112 and may be accessible to training server system 140 over one or more networks (not shown) for training the machine learning model(s).

The training data refers to a dataset that has been featurized and labeled. For example, the dataset may include a plurality of data records, each including information corresponding to a different host profile stored in host database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a host, which is reflected in a corresponding data record, may be a feature used in training the machine learning model. Such features may include demographic information (e.g., age, gender, ethnicity, etc.), analyte information (e.g., normal lactate ranges (e.g., lactate baseline, lactate threshold, lactate clearance rate, and/or lactate rate of change during and after exercise, etc.), exercise related information (e.g., start and end times associated with exercise, duration of exercise, exercise type and/or intensity (e.g., speed, incline, resistance, etc.), non-analyte sensor information (e.g., heart rate, temperature, etc.), liver health information (e.g., liver disease diagnosis and staging), comorbidities (e.g., kidney disease), and/or any other information relevant to classifying hosts and/or optimizing exercise for the hosts. In addition, the data record is labeled with information the corresponding model is being trained to predict. In one example, if a model is being trained to classify a host into a healthy host, an athlete, or a metabolically unfit host, then the data records in the training dataset are labeled with such classification. In another example, if a model is being trained to output optimized exercise parameters, then the data records in the training dataset are labeled with one or more of such parameters. Note that, in one example, such a model may be a multi-input single-output (MISO) model, configured to predict only one optimized exercise parameter (e.g., speed), in which case additional MISO models may be trained for each predicting one of other exercise parameters (e.g., incline, resistance, etc.). In another example, such a model may be a multi-input multi-output (MIMO) model, configured to predict multiple optimized exercise parameters (e.g., speed, incline, resistance, etc.).

The model(s) are then trained by training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical host, the model(s) may be iteratively refined to generate accurate predictions of a host's classification, optimized exercise parameters, etc.

As illustrated in FIG. 1, training server system 140 deploys these trained model(s) to therapy management engine 114 for use during runtime. For example, therapy management engine 114 may obtain host profile 118 associated with a host and stored in host database 110, use information in host profile 118 as input into the trained model(s), and output a prediction indicative of the host's classification as a metabolically unfit host or otherwise, and/or suggested exercise parameters for HIIT, resistance training, or Zone 2 exercise (e.g., shown as output 144 in FIG. 1). Output 144 generated by therapy management engine 114 may also indicate improvement in the host's metabolic health over time. Output 144 may be provided to the host (e.g., through application 106), to a caretaker of the host (e.g., a parent, a relative, a guardian, a teacher, a physical therapist, a fitness trainer, a nurse, etc.), to a physician or healthcare provider of the host, or any other individual that has an interest in the wellbeing of the host for purposes of improving the health of the host, such as, in some cases by effectuating recommended treatment. Output 144 generated by therapy management engine 114 is stored in host database 110 and is utilized to train or re-train the trained model(s).

In certain embodiments, output 144 generated by therapy management engine 114 may be stored in host profile 118. Output 144 may be indicative of a host classification (e.g., healthy host, athlete, or metabolically unfit), current or future metabolic fitness level, optimized exercise parameters, etc. Output 144 stored in host profile 118 may be continuously updated by therapy management engine 114. Accordingly, for example, previous metabolic fitness predictions and disease diagnoses, originally stored as outputs 144 in host profile 118 in host database 110 and then passed to historical records database 112, may provide an indication of the progression or improvement of the metabolic fitness of a host over time, as well as provide an indication as to the effectiveness of different exercise recommendations and/or treatments recommended to the host to improve metabolic fitness.

In certain embodiments, a host's own historical data may be used by training server system 140 to train a personalized model for the host that provides therapy management support and insight around the host's exercise goals, exercise optimization, and the host's metabolic fitness. For example, in certain embodiments, a model trained based on population data may be used to provide optimized exercise parameters to the host. However, after collecting personalized information (e.g., analyte sensor information, non-analyte sensor information, exercise type, and/or parameters, etc.) associated with one or more exercise sessions performed by the host, the personalized information may be used for further personalizing the model. For example, information obtained from one or more exercise sessions performed by the host (e.g., including information obtained during the one or more exercise sessions as well as information obtained after the one or more exercise sessions reflecting the impact of such one or more sessions) may be used to optimize exercise parameters for future exercise sessions.

Further, a host's historical data may be used as a baseline to indicate improvements or deterioration in the host's metabolic fitness based, for example, on the host's ability to clear lactate following an exercise session. As an illustrative example, a host's data from 2 weeks ago may be used as a baseline that can be compared with the host's current data to identify whether the host's metabolic fitness has improved. In certain embodiments, the model may further be able to predict or project out the host's metabolic fitness or its future improvement/deterioration based on the host's recent pattern of data (e.g., exercise data, food consumption data, etc.).

In certain embodiments, a model may be trained to provide food, lifestyle, and other types of therapy management support recommendations to help the host improve their metabolic fitness based on the host's historical data, including how different types of food and/or activities impacted the host's metabolic fitness in the past. In certain embodiments, a model may be trained to predict the underlying cause of certain improvements or deteriorations in the host's metabolic fitness. For example, application 106 may display a user interface with a graph that shows the host's metabolic fitness (e.g., liver function) with trend lines and indicate, e.g., retrospectively, how the liver functionality suffered at certain points in time.

FIG. 2 is a diagram 200 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a host, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240, as well as an exercise machine (e.g., exercise machine 108 of FIG. 1). In certain embodiments, sensor electronics module 204 may also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 may comprise one or more sensors for detecting and/or measuring analyte(s). The continuous analyte sensor 202 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 202 may be configured to continuously measure analyte levels of a host using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 202 provides a data stream indicative of the concentration of one or more analytes in the host. The data stream may include raw data signals, which are then converted into a calibrated and/or filtered data stream used to provide estimated analyte value(s) to the host.

In certain embodiments, the continuous analyte sensor 202 may be a multi-analyte sensor, configured to continuously measure multiple analytes in a host's body. For example, in certain embodiments, the continuous multi-analyte sensor 202 may be a single sensor configured to measure lactate, glucose, ketones (e.g., 3-beta-hydroxybutyrate, acetoacetate, acetone, etc.), glycerol, and/or free fatty acids in the host's body.

In certain embodiments, one or more multi-analyte sensors may be used in combination with one or more single analyte sensors. As an illustrative example, a multi-analyte sensor may be configured to continuously measure lactate and glucose and may, in some cases, be used in combination with an analyte sensor configured to measure only ketones or only potassium. Information from each of the multi-analyte sensor(s) and single analyte sensor(s) may be combined to provide therapy management support using methods described herein. In further embodiments, other non-contact and or periodic or semi-continuous, but temporally limited, measurements for physiological information may be integrated into the system such as by including weight scale information or non-contact heart rate monitoring from a sensor pad under the host while in a chair or bed, through an infra-red camera detecting temperature and/or blood flow patterns of the host, and/or through a visual camera with machine vision for height, weight, or other parameter estimation without physical contact.

In certain embodiments, the continuous analyte sensor(s) 202 may comprise a percutaneous wire that has a proximal portion coupled to the sensor electronics module 204 and a distal portion with several electrodes, such as a measurement electrode and a reference electrode. The measurement (or working) electrode may be coated, covered, treated, embedded, etc., with one or more chemical molecules that react with a particular analyte, and the reference electrode may provide a reference electrical voltage. The measurement electrode may generate the analog electrical signal, which is conveyed along a conductor that extends from the measurement electrode to the proximal portion of the percutaneous wire that is coupled to the sensor electronics module 204. After the continuous analyte monitoring system 104 has been applied to epidermis of the patient, continuous analyte sensor(s) 202 penetrates the epidermis, and the distal portion extends into the dermis and/or subcutaneous tissue under epidermis. Other configurations of continuous analyte sensor(s) 202 may also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 202 may include a single-analyte sensor configured to measure lactate concentration levels, and another single-analyte sensor configured to measure glucose concentration levels of the patient. As another illustrative example, continuous analyte sensor(s) 202 may include a single-analyte sensor configured to measure lactate concentration levels, and one or more multi-analyte sensors configured to measure glucose concentration levels, ketone concentration levels, creatinine concentration levels, etc. As yet another illustrative example, continuous analyte sensor(s) 202 may include a multi-analyte sensor configured to measure lactate concentration levels, glucose concentration levels, ketone concentration levels, creatinine concentration levels, etc. Accordingly, continuous analyte sensor(s) 202 is configured to generate at least one analog electrical signal that is proportional to the concentration level of a particular analyte, and sensor electronics module 204 is configured to convert the analog electrical signal into an analyte sensor count values, calibrate the analyte sensor count values based on the sensitivity profile of the continuous analyte sensor(s) 202 to generate measured analyte concentration levels, and transmit the measured analyte concentration level data, including the measured analyte concentration levels, to a display device, such as display devices 210, 220, and/or 230, via a wireless connection. For example, sensor electronics module 204 may be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 202 may incorporate a thermocouple within, or alongside, the percutaneous wire to provide an analog temperature signal to the sensor electronics module 204, which may be used to correct the analog electrical signal or the measured analyte data for temperature. In other embodiments, the thermocouple may be incorporated into the sensor electronics module 204 above the adhesive pad, or, alternatively, the thermocouple may contact the epidermis of the patient through openings in the adhesive pad.

In certain embodiments, the sensor electronics module 204 includes, inter alia, processor 233, storage element or memory 234, wireless transmitter/receiver (transceiver) 236, one or more antennas coupled to wireless transceiver 236, analog electrical signal processing circuitry, analog to-digital (A/D) signal processing circuitry, digital signal processing circuitry, a power source for continuous analyte sensor(s) 202 (such as a potentiostat), etc.

Processor 233 may be a general-purpose or application-specific microprocessor, an application-specific integrated circuit (ASIC), a field programmable gate array (FPGA), etc., that executes instructions to perform control, computation, input/output, etc. functions for the sensor electronics module 204. Processor 233 may include a single integrated circuit, such as a micro processing device, or multiple integrated circuit devices and/or circuit boards working in cooperation to accomplish the appropriate functionality. In certain embodiments, processor 233, memory 234, wireless transceiver 236, the A/D signal processing circuitry, and the digital signal processing circuitry may be combined into a system-on-chip (SoC).

Generally, processor 233 may be configured to sample the analog electrical signal using the A/D signal processing circuitry at regular intervals (such as the sampling period) to generate analyte sensor count values based on the analog electrical signals produced by the continuous analyte sensor(s) 202, calibrate the analyte sensor count values based on the sensitivity profile of the continuous analyte sensor(s) 202 to generate measured analyte concentration levels, and generate measured analyte data from the measured analyte concentration levels, generate sensor data packages that include, inter alia, the measured analyte concentration level data. Processor 233 may store the measured analyte concentration level data in memory 234, and generate the sensor data packages at regular intervals (such as the transmission period) for transmission by wireless transceiver 236 to a display device, such as display devices 210, 220, 230, and/or 240. Processor 233 may also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or Bluetooth Low Energy (BLE) connection. In such embodiments, the sensor data packages are transmitted in the form of Bluetooth or BLE data packets to the display device

In various embodiments, memory 234 may include volatile and nonvolatile medium. For example, memory 234 may include combinations of random access memory (RAM), dynamic RAM (DRAM), static RAM (SRAM), read only memory (ROM), flash memory, cache memory, and/or any other type of non-transitory computer-readable medium. Memory 234 may store one or more analyte sensor system applications, modules, instruction sets, etc. for execution by processor 233, such as instructions to generate measured analyte data from the analyte sensor count values, etc.

Memory 234 may also store certain sensor operating parameters 235, such as a calibration slope (or calibration sensitivity), a calibration baseline, etc. In particular, the calibration sensitivity, calibration baseline, and other information related to the sensitivity profile for the sensor electronics module 204 may be programmed into the sensor electronics module 204 during the manufacturing process, and then used to convert the analyte sensor electrical signals into measured analyte concentration levels. For example, as discussed above, the calibration slope may be used to predict an initial in vivo sensitivity (M₀) and a final in vivo sensitivity (M_{f}), which are stored in memory 234 and used to convert the analyte sensor electrical signals into measured analyte concentration levels. In certain embodiments, calibration sensitivity (M_{CC}) 246 and/or calibration baseline 247 may be stored in memory 234.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 may include hardware, firmware, and/or software that enable measurement of levels of analyte(s) via continuous analyte sensor(s) 202. For example, sensor electronics module 204 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Each of display devices 210, 220, 230, or 240 may include a display such as a touchscreen display 212, 222, 232, and/or 242 for displaying sensor data to a host and/or for receiving inputs from the host. For example, a graphical user interface (GUI) may be presented to the host for such purposes. In certain embodiments, the display devices may include other types of user interfaces such as a voice user interface instead of, or in addition to, a touchscreen display for communicating sensor data to the host of the display device and/or for receiving host inputs. Display devices 210, 220, 230, and 240 may be examples of display device 107 illustrated in FIG. 1 used to display sensor data to a host of the system of FIG. 1 and/or to receive input from the host. In certain embodiments, exercise machine 108 may also function as a display device for communicating sensor data to the host of the display device and/or for receiving host inputs.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 210 is an example of such a custom device. In certain embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch or fitness tracker, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end host) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data.

As mentioned, sensor electronics module 204 may be in communication with a medical device 208. Medical device 208 may be a passive device in some example embodiments of the disclosure. For example, medical device 208 may be an insulin pump for administering insulin to a host. For a variety of reasons, it may be desirable for such an insulin pump to receive and track lactate, glucose, ketones, glycerol and free fatty acid values transmitted from continuous analyte monitoring systems 104, where continuous analyte sensor 202 is configured to measure lactate, glucose, ketones, glycerol, and/or free fatty acids.

Further, as mentioned, sensor electronics module 204 may also be in communication with other non-analyte sensors 206. Non-analyte sensors 206 may include, but are not limited to, an altimeter sensor, an accelerometer sensor, a global positioning system (GPS) sensor, a temperature sensor, a respiration rate sensor, etc. Non-analyte sensors 206 may also include monitors such as heart rate monitors, blood pressure monitors, pulse oximeters, caloric intake monitors, indirect calorimetry devices and medicament delivery devices. One or more of these non-analyte sensors 206 may provide data to therapy management engine 114 described further below. In some aspects, a host may manually provide some of the data for processing by training server system 140 and/or therapy management engine 114 of FIG. 1.

In certain embodiments, non-analyte sensors 206 may further include sensors for measuring skin temperature, core temperature, sweat rate, and/or sweat composition.

In certain embodiments, the non-analyte sensors 206 may be combined in any other configuration, such as, for example, combined with one or more continuous analyte sensors 202. As an illustrative example, a non-analyte sensor, e.g., a temperature sensor, may be combined with a continuous lactate sensor 202 to form a lactate/temperature sensor used to transmit sensor data to the sensor electronics module 204 using common communication circuitry. As another illustrative example, a non-analyte sensor, e.g., a temperature sensor, may be combined with a multi-analyte sensor 202 configured to measure lactate and glucose to form a lactate/glucose/temperature sensor used to transmit sensor data to the sensor electronics module 204 using common communication circuitry.

In certain embodiments, a wireless access point (WAP) may be used to couple one or more of continuous analyte monitoring system 104, the plurality of display devices, medical device(s) 208, and/or non-analyte sensor(s) 206 to one another. For example, WAP 138 may provide Wi-Fi and/or cellular connectivity among these devices. Near Field Communication (NFC) and or Bluetooth may also be used among devices depicted in diagram 200 of FIG. 2.

FIG. 3 illustrates example inputs and example metrics that are calculated based on the inputs for use by the therapy management system of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and DAM 116 in the middle, and metrics 130 on the right. In certain embodiments, each one of metrics 130 may correspond to one or more values, e.g., discrete numerical values, ranges, or qualitative values (high/medium/low, stable/unstable, etc.). Application 106 obtains inputs 128 through one or more channels (e.g., manual host input, sensors, other applications executing on display device 107, an EMR system, etc.). As mentioned previously, in certain embodiments, inputs 128 may be processed by DAM 116 to output a plurality of metrics, such as metrics 130. Inputs 128 and metrics 130 may be used by training server system 140 and therapy management engine 114 to both train and deploy one or more machine learning models for classifying hosts, optimizing exercise parameters to improve metabolic fitness of metabolically unfit hosts, and other functionalities described herein.

In certain embodiments, starting with inputs 128, host statistics, such as one or more of age, height, weight, BMI, body composition (e.g., % body fat or % muscle from a computed tomography (CT) scan, a magnetic resonance imaging (MRI) scan, dual-energy X-ray absorptiometry (DEXA) scan, etc.), stature, build, or other information may also be provided as an input. In certain embodiments, host statistics are provided through a user interface, by interfacing with an electronic source such as an electronic medical record, and/or from measurement devices. In certain embodiments, the measurement devices include one or more wireless devices, e.g., Bluetooth-enabled, weight scale and/or camera, which may, for example, communicate with the display device 107 or exercise machine 108 to provide host data.

In certain embodiments, treatment/medication information is also provided as an input. Medication information may include information about the type, dosage, and/or timing of when one or more medications are to be taken by the host. Treatment information may include information regarding different lifestyle habits recommended by the host's physician. For example, the host's physician may recommend a host follow specific diet recommendations, exercise for a minimum of thirty minutes a day, or cut calories by 500 to 1,000 calories daily to improve liver health. In certain embodiments, treatment/medication information may be provided through manual host input.

In certain embodiments, analyte sensor data may also be provided as input, for example, through continuous analyte monitoring system 104. In certain embodiments, analyte sensor data may include lactate data (e.g., a host's lactate values) measured by at least a lactate sensor (or multi-analyte sensor) in continuous analyte monitoring system 104. In certain embodiments, analyte sensor data may include glucose data measured by at least a glucose sensor (or multi-analyte sensor) in continuous analyte monitoring system 104. In certain embodiments, analyte sensor data may include ketone data measured by at least a ketone sensor (or multi-analyte sensor) in continuous analyte monitoring system 104. In certain embodiments, analyte sensor data may include potassium data measured by at least a potassium sensor (or multi-analyte sensor) in continuous analyte monitoring system 104. In certain embodiments, analyte sensor data may include creatinine data measured by at least a creatinine sensor (or multi-analyte sensor) in continuous analyte monitoring system 104. In certain embodiments, analyte sensor data may include a determination of a creatinine to cystatin C ratio for the host. In certain embodiments, analyte sensor data may include point-in-time creatinine data and/or point-in-time creatinine to cystatin C ratio.

In certain embodiments, input may also be received from one or more non-analyte sensors, such as non-analyte sensors 206 described with respect to FIG. 2. Input from such non-analyte sensors 206 may include information related to a heart rate, a respiration rate, oxygen saturation, blood pressure, or a body temperature (e.g. to detect illness, physical activity, etc.) of a host. In certain embodiments, electromagnetic sensors may also detect low-power radio frequency (RF) fields emitted from objects or tools touching or near the object, which may provide information about host activity or location.

In certain embodiments, input received from non-analyte sensors may include input relating to a host's insulin delivery. In particular, input related to the host's insulin delivery may be received, via a wireless connection on a smart pen, via host input, and/or from an insulin pump. Insulin delivery information may include one or more of insulin volume, time of delivery, etc. Other parameters, such as exogenous insulin action time or duration of exogenous insulin action, may also be received as inputs.

In certain embodiments, starting with inputs 128, food consumption information may include information about one or more of meals, snacks, and/or beverages, such as one or more of the size, content (carbohydrate, fat, protein, etc.), sequence of consumption, and time of consumption. In certain embodiments, food consumption may be provided by a host through manual entry, by providing a photograph through an application that is configured to recognize food types and quantities, and/or by scanning a bar code or menu. In various examples, meal size may be manually entered as one or more of calories, quantity (“three cookies”), menu items (“Royale with Cheese”), and/or food exchanges (1 fruit, 1 dairy). In some examples, meal information may be received via a convenient user interface provided by application 106 and/or application 111.

In certain embodiments, food consumption information (the type of food (e.g., liquid or solid, snack or meal, etc.) and/or the composition of the food (e.g., carbohydrate, fat, protein, etc.)) may be determined automatically based on information provided by one or more sensors. Some example sensors may include body sound sensors (e.g., abdominal sounds may be used to detect the types of meal, e.g., liquid/solid food, snack/meal, etc.), radio-frequency sensors, cameras, hyperspectral cameras, and/or analyte (e.g., insulin, glucose, lactate, etc.) sensors to determine the type and/or composition of the food.

In certain embodiments, food consumption entered by a host may relate to lactate consumed by the host. Lactate for consumption may include any natural or designed food or beverage that contains lactate (e.g., a lactate drink, yogurt, or whole milk) or that produces lactate when consumed (e.g., fructose drink). As will be described in more detail with respect to metrics 130 computed by DAM 116, such lactate consumption information may be used by DAM 116 to calculate lactate clearance rates of the host.

In certain embodiments, exercise information is also provided as an input. Exercise information may be any information surrounding activities requiring physical exertion by the host. For example, exercise information may range from information related to low intensity (e.g., walking a few steps) and high intensity (e.g., five mile run) physical exertion. In certain embodiments, the exercise information may comprise information related to HIIT, resistance training, or Zone 2 training. In certain embodiments, exercise information may also be provided through manual host input suggesting the host will begin a specific exercise type and/or with certain exercise parameters. In certain embodiments, exercise information may also be provided by exercise machine 108, including the type, duration, and parameters of the exercise. In certain embodiments, exercise information may be provided or determined based on information provided, for example, by non-analyte sensors 206 (e.g., a temperature sensor, a heart rate monitor, a wearable blood pressure monitor, an accelerometer sensor on a wearable device such as a watch, fitness tracker, and/or patch, etc.). In certain embodiments, exercise information may be provided or determined based on information provided, for example, by continuous analyte sensor system 104 (e.g., it may be deduced that the host engaged in exercise based on their lactate, glucose, potassium, and/or ketone data). The exercise information provided by analyte and non-analyte sensors may be used as input into a model trained for predicting whether the host is engaging in exercise and/or predicting the types and/or parameters of such exercise.

When predicting that a host is exercising based on his/her analyte and non-analyte sensor data, the host may be asked to confirm if exercise is occurring, what type of exercise, and or the level of strenuous exertion being used during the exercise over a specific period. This data may be used as feedback to re-train the model to learn about the host's exercise patterns and to reduce the need for confirmation questions as time progresses and the model becomes optimized. Other data, such as time and day, location, etc. may similar be used as input into such a model for making exercise related predictions.

In certain embodiments, time may also be provided as an input, such as time of day or time from a real-time clock. For example, in certain embodiments, input analyte data may be timestamped to indicate a date and time when the analyte measurement was taken for the host.

Host input of any of the above-mentioned inputs 128 may be provided through continuous analyte sensor system 104, non-analyte sensors 206, and/or a user interface, such a user interface of display device 107 or exercise machine 108 of FIG. 1. As described above, in certain embodiments, DAM 116 determines or computes the host's metrics 130 based on inputs 128. An example list of metrics 130 is shown in FIG. 3.

In certain embodiments, lactate levels may be determined from sensor data (e.g., lactate measurements obtained from a continuous lactate sensor of continuous analyte monitoring system 104). For example, lactate levels refer to time-stamped lactate measurements or values that are continuously generated and stored over time.

In certain embodiments, lactate production rates may be determined from sensor data (e.g., lactate measurements obtained from a continuous lactate sensor of continuous analyte monitoring system 104). In particular, lactate is produced from pyruvate (e.g., glucose is broken down to pyruvate) through enzyme lactate dehydrogenase during normal metabolism and exercise. In certain embodiments, a lactate production rate may be determined by assessing an increase in lactate levels over a specified amount of time. In certain embodiments, lactate production rates may be expressed as a percentage of a maximum heart rate (e.g., 85% of maximum heart rate) or a percentage of a maximum oxygen intake (e.g., 75%). In certain other embodiments, lactate production rates may be expressed as a function of accelerometer data. For example, accelerometer data may indicate a step rate of a host over time (e.g., increasing step rate shown by increasing accelerometer data and vice versa). Each of these step rates may correlate to a lactate level of a host a specified time. Thus, step rates analyzed over time (e.g., accelerometer data) and their corresponding lactate levels may provide information about a host's lactate production rate with respect to accelerometer data. DAM 116 may continuously, semi-continuously, or periodically measure a host's lactate production rate over time and store the lactate production rates with time-stamps in the host's profile 118. Lactate production rates may be time-stamped to allow for identifying a decrease or increase of the host's lactate production over time.

In certain embodiments, a lactate baseline may be determined from sensor data (e.g., lactate measurements obtained from a continuous lactate sensor of continuous analyte monitoring system 104). A lactate baseline represents a host's normal lactate levels during periods where fluctuations in lactate production is typically not expected. A host's baseline lactate is generally expected to remain constant over time, unless challenged through an action such as the consumption of a food or beverage that contains lactate or that produces lactate, or exercise by the host. Additionally, a host's baseline lactate may also change based on the host's health, specifically in response to an improvement or decline in liver health. Further, each host may have a different lactate baseline. In certain embodiments, a host's lactate baseline may be determined by calculating an average of lactate levels over a specified amount of time where fluctuations are not expected.

For example, the baseline lactate for a host may be determined over a period of time when the host is sleeping, sitting in a chair, or other periods of time where the host is sedentary and not consuming food or medication which would reduce or increase lactate levels. In certain embodiments, DAM 116 may continuously, semi-continuously, or periodically calculate a lactate baseline and time-stamp and store the corresponding information in the host's profile 118. In certain embodiments, DAM 116 may calculate the lactate baseline using lactate levels measured over a period of time where the host is sedentary, the host is not consuming lactate, and where no external conditions exist that would affect the lactate baseline. In certain other embodiments, DAM 116 may use lactate levels measured over a period of time where the host is, at least for a subset of the period of time, engaging in exercise and/or consuming lactate and/or an external condition exists that would affect the lactate baseline. In this case, in some examples, DAM 116 may first identify which measured lactate values are to be used for calculating the baseline lactate by identifying lactate values that may have been affected by an external event, such the consumption of food, exercise, medication, or other perturbation that would disrupt the capture of a lactate baseline measurement. DAM 116 may then exclude such measurements when calculating the lactate baseline of the host. In some other examples, DAM 116 may calculate the lactate baseline by first determining a percentage of the number of lactate values measured during this time period that represent the lowest lactate values measured. DAM 116 may then take an average of this percentage to determine the lactate baseline level.

In certain embodiments, a lactate clearance rate may be determined from sensor data (e.g., lactate measurements obtained from a continuous lactate sensor of continuous analyte monitoring system 104). In particular, a host's lactate clearance rate indicates the rate at which lactate production is greater than lactate consumption. A lactate clearance rate may be indicative of liver function or metabolic fitness (e.g., the slope of a curve of lactate clearance may indicate liver function or metabolic fitness). In certain embodiments, the lactate clearance rate may be determined by calculating a slope between an initial lactate value (e.g., during a period of increased lactate levels) and a lactate baseline associated with the host. In certain embodiments, a lactate clearance rate may be calculated over time until the lactate levels of the host reach some value relative to the host's lactate baseline (e.g., 50% or 75% of lactate baseline). In certain embodiments, a lactate clearance rate may be calculated over time until the lactate levels of the host reach some value relative to a peak lactate level measured for the host at a previous time (e.g., lactate levels of the host reach 25%, 50%, and/or 75% of a peak lactate level of the host).

In certain embodiments, a lactate clearance rate may be expressed as a function of lactate half-life of a host. In particular, an inverse relationship exists between the lactate clearance rate and lactate half-life. In a diseased liver, the slope of lactate clearance is reduced as the calculated lactate half-life increases. As liver disease progresses, the slope of lactate clearance is further reduced and the calculated lactate half-life further increases. Thus, lactate half-life may be indicative of the lactate clearance rate of a host. Lactate clearance rates calculated over time may be time-stamped and stored in the host's profile 118.

In certain embodiments, a lactate trend may be determined based on lactate levels over certain periods of time. In certain embodiments, lactate trends may be determined based on lactate production rates over certain periods of time. In certain embodiments, lactate trends may be determined based on calculated lactate clearance rates over certain periods of time.

In certain embodiments, a lactate threshold may be determined from sensor data (e.g., lactate measurements obtained from continuous analyte monitoring system 104). A lactate threshold may be indicative of the lactate value of a host at which lactate production exceeds lactate clearance, which may be caused by the host engaging in high intensity, anaerobic activity. Each host may have a different lactate threshold. In certain embodiments, a lactate threshold may be determined by determining a lowest lactate value during a specified amount of time where lactate levels increase exponentially (i.e., rapidly). In certain embodiments, lactate threshold may be determined by determining a highest lactate value before an increasing work rate of the host leads to exponentially increasing lactate levels. In certain embodiments, a lactate threshold for a host may be determined over a period of time when the host is engaging in physical activity, such as Zone 2, resistance training, or HIIT. In certain embodiments, DAM 116 may continuously or periodically calculate a lactate threshold and time-stamp and store the corresponding information in the host's profile 118.

In certain embodiments, an intensity at the lactate threshold may be determined from sensor data (e.g. lactate measurements obtained from continuous analyte monitoring system 104) combined with exercise and/or non-analyte sensor data. The intensity at the lactate threshold may be the intensity (e.g. power, speed, wattage, etc.) at which the lactate threshold is met. For example, the lactate threshold may be the intensity just before the host's lactate production begins to exceed lactate clearance, as measured by a continuous lactate sensor of continuous analyte monitoring system 104. In certain embodiments, the intensity at the lactate threshold may be determined over a period of time when the host is engaging in physical activity, such as Zone 2, resistance training, or HIIT. In certain embodiments, DAM 116 may continuously or periodically calculate an intensity when or just prior to the lactate threshold being reached and time-stamp and store the corresponding information in the host's profile 118.

In certain embodiments, a starting lactate level at the outset of an exercise session may be determined from sensor data (e.g. lactate measurements obtained from continuous analyte monitoring system 104) combined with exercise and/or non-analyte sensor data. The starting lactate level at the outset of exercise may be the host's lactate level as the host is beginning an exercise session. In certain embodiments, the starting lactate level may be determined based on the level of lactate at the start of exercise, before the host's lactate begins to increase and/or decrease, or when the host inputs information related to an exercise session beginning. In certain embodiments, the starting lactate at the outset of an exercise session may be determined over a period of time when the host is engaging in exercise, such as Zone 2, resistance training, or HIIT. In certain embodiments, DAM 116 may continuously or periodically calculate a starting lactate level at the outset of an exercise session and time-stamp and store the corresponding information in the host's profile 118.

In certain embodiments, glucose levels may be determined from sensor data (e.g., blood glucose measurements obtained from a continuous lactate sensor of continuous analyte monitoring system 104).

In certain embodiments, a blood glucose trend may be determined based on glucose levels over a certain period of time.

In certain embodiments, insulin sensitivity may be determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a host's cells are to insulin. Improving insulin sensitivity for a host may help to reduce insulin resistance in the host.

In certain embodiments, insulin on board may be determined using non-analyte sensor data input (e.g., insulin delivery information) and/or known or learned (e.g. from host data) insulin time action profiles, which may account for both basal metabolic rate (e.g., update of insulin to maintain operation of the body) and insulin usage driven by activity or food consumption.

In certain embodiments, health and sickness metrics may be determined, for example, based on one or more of host input (e.g., pregnancy information or known sickness information), from physiologic sensors (e.g., temperature), activity sensors, or a combination thereof. In certain embodiments, based on the values of the health and sickness metrics, for example, a host's state may be defined as being one or more of healthy, ill, rested, or exhausted.

In certain embodiments, disease stage metrics, such as for liver disease, may be determined, for example, based on one or more of host input or output provided by therapy management engine 114 illustrated in FIG. 1. In certain embodiments, example disease stages for liver disease can include an inflammation stage (e.g., early stage where the host's liver is enlarged or inflamed), a fibrosis stage (e.g., stage with signs of scar tissue in the inflamed liver), a cirrhosis stage (e.g., stage with signs of severe scar tissue in the inflamed liver), and an end-stage liver disease (ESLD). In certain embodiments, example disease stages may be represented as a NASH score, an NAFLD fibrosis score, a Child-Pugh score, a model for ESLD (MELD) score, a meta-analysis of histological data in viral hepatitis (METAVIR) score, and the like. Additionally, hepatocellular carcinoma may often be present throughout the later stages of cirrhosis and or ESLD.

In certain embodiments, the meal state metric may indicate the state the host is in with respect to food consumption. For example, the meal state may indicate whether the host is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first).

In certain embodiments, meal habits metrics are based on the content and the timing of a host's meals. For example, if a meal habit metric is on a scale of 0 to 1, the better/healthier meals the host cats the higher the meal habit metric of the host will be to 1, in an example. Also, the more the host's food consumption adheres to a certain time schedule, the closer their meal habit metric will be to 1, in the example. In certain embodiments, the meal habit metrics may indicate whether a host has been consistently participating in a ketogenic diet (e.g., a low-carb, moderate protein, and higher-fat diet) based on meals, snacks, or beverages consumed by the host over a certain period of time.

In certain embodiments, the activity level metric may indicate the host's level of activity. In certain embodiments, the activity level metric be determined, for example based on input from an activity sensor or other physiologic sensors, such as non-analyte sensors 206. In certain embodiments, the activity level metric may be calculated by DAM 116 based on one or more of inputs 128, such as one or more of exercise information, non-analyte sensor data (e.g., accelerometer data), time, host input, etc. In certain embodiments, the activity level may be expressed as a step rate of the host. Activity level metrics may be time-stamped so that they can be correlated with the host's lactate levels at the same time.

In certain embodiments, exercise regimen metrics may indicate one or more of what type of activities the host engages in, the corresponding intensity of such activities, frequency the host engages in such activities, etc. In certain embodiments, exercise regimen metrics may be calculated based on one or more of analyte and/or non-analyte sensor data input (e.g., non-analyte sensor data input from an accelerometer, a heart rate monitor, a blood pressure monitor, a respiration rate sensor, etc.), calendar input, host input, etc.

In certain embodiments, metabolic rate is a metric that may indicate or include a basal metabolic rate (e.g., energy consumed at rest) and/or an active metabolism (e.g., energy consumed by activity, such as physical exertion). In some examples, basal metabolic rate and active metabolism may be tracked as separate outcome metrics. In certain embodiments, the metabolic rate may be calculated by DAM 116 based on one or more of inputs 128, such as one or more of exercise information, analyte sensor data, non-analyte sensor data, time, etc. In certain embodiments, the metabolic rate may be calculated and metabolic rates calculated over time may be time-stamped and stored in the host's profile 118.

In certain embodiments, metabolic fitness is a metric measuring liver function and/or ability of the liver and skeletal muscle to clear lactate efficiently. Metabolic fitness may be determined, for example, by considering the host's baseline lactate, starting lactate level at the outset of exercise, lactate concentration at various exercise intensities, and lactate clearance rate over certain periods of time and comparing these metrics to defined (e.g., population-based) threshold and/or ranges of baseline lactate, starting lactate level at the outset of exercise, lactate concentration at various exercise intensities, and lactate clearance rate metrics corresponding to various levels of metabolic fitness (e.g., healthy hosts, metabolically unfit hosts, athletes, etc.). Further, improvement or deterioration in metabolic fitness can be determined by comparing the host's current baseline lactate, starting lactate level at the outset of exercise, and lactate clearance rate over certain periods of time to the host's past baseline lactate, starting lactate level at the outset of exercise, and lactate clearance rate over certain periods of time. In certain embodiments, the metabolic fitness may be calculated over time may be time-stamped and stored in the host's profile 118.

In certain embodiments, fat loss metrics may be calculated by DAM 116 based on inputs 128, and more specifically, non-analyte sensor data such as indirect calorimetry. Indirect calorimetry may assess energy expenditure of the host and predict carbohydrate, lipid and protein utilization based on the overall energy expenditure. Based on the utilization of macronutrients (e.g. protein, fat or carbohydrates), fat loss metrics may be predicted based on an exercise session. Further, fat loss metrics may inform the host of what macronutrients to consume following an exercise session for best results (e.g. the host should consume protein but not fat following an exercise session.

In certain embodiments, potassium levels and metrics may be calculated by DAM 116 based on inputs 128. Potassium levels may be determined from sensor data (e.g. potassium measurements obtained from continuous analyte monitoring system 104). Potassium levels may also be used to determine potassium metrics such as an absolute maximum potassium level, an individualized maximum potassium level, a duration of time where potassium is above a certain threshold, an individualized Zone 2 potassium range and/or a potassium rate of change.

In certain embodiments, the absolute maximum potassium level represents a host's maximum potassium level determined to be unsafe over a period of time (e.g., hourly, weekly, daily, etc.). In certain embodiments, the absolute maximum potassium level may be consistent across all hosts (e.g., set to 5.5 mmol/L based on current medical guidelines). In certain other embodiments, each patient may have a different absolute maximum potassium level (e.g., an individualized maximum potassium level). In certain embodiments, the duration of time where potassium is above a certain threshold may be the duration of time when potassium is above the absolute maximum potassium level across all hosts, or the individualized maximum potassium level. In certain embodiments, the individualized Zone 2 potassium range may be a potassium range indicative of a Zone 2 exercise intensity for the host based on a host's historical data. In certain embodiments, a potassium level rate of change refers to a rate that indicates how one or more time-stamped potassium measurements or values change in relation to one or more other time-stamped potassium measurements or values. Potassium level rates of change may be determined over one or more seconds, minutes, hours, days, etc.

Generally different hosts have different physiology, and therefore optimizing an exercise session may require different exercise parameters for the host to reach their goals. For example, exercise parameters that may be effective for metabolically unfit hosts with a goal of improving metabolic fitness may not be effective for an athlete with a goal of increasing peak performance. As such, as described in relation to FIG. 4, classifying the type of host prior to optimizing exercise for a host (e.g., providing optimized exercise guidance and/or controlling operations of an exercise machine) ensures that the exercise parameters will be effective for a specific host to reach their specific goals.

FIG. 4 is a flow diagram depicting an example method 400 for classifying a host as a healthy host, an athlete, or a metabolically unfit host, according to certain embodiments of the present disclosure. In certain embodiments, the workflow 400 classifies the host, e.g., a host illustrated in FIG. 1, using at least a continuous lactate monitor.

Workflow 400 may be performed by therapy management system 100, including therapy management engine 114. As shown in FIG. 4, workflow 400 begins at block 402, by therapy management engine 114 receiving input data (i.e., inputs) to classify the host. The inputs may be received in a variety of ways. For example, the inputs may be received or retrieved from the host profile 118, which includes demographic info 120, disease info 122, medication info 124, inputs 128, metrics 130, etc. Inputs may also be received as host input through the user interface of a display device 107, user interface 113, and/or an exercise machine 108.

For example, the host may be asked for and provide a host classification (e.g. the opinion of the host regarding whether the host is a metabolically unfit host, a healthy host, or an athlete), host goals related to health or exercise, medical history of the host including diabetes or liver disease, demographic information of the host (age, gender, ethnicity etc.), host's historical exercise data, and metabolic adaptations, etc. In certain embodiments, demographic information may be relevant in determining the metabolic fitness of the host (e.g. the host's ability to clear lactate and glucose after a meal or exercise, and/or liver disease stage). For example, age may be indicative of metabolic fitness, as metabolic fitness changes with age. In another example, sarcopenic adults (e.g., those who have experienced skeletal muscle degradation with age) suffer from a lower metabolic fitness as there is less skeletal muscle to process blood lactate when compared to non-sarcopenic adults.

In certain embodiments, metabolic adaptations of the host such as AMP-activated protein kinase (AMPK) activation may be used to determine the host's ability to consume glucose and oxidize fat and, in turn, to classify the host. When AMPK is activated, it regulates cellular metabolism which stimulates glucose uptake and lipid oxidation to produce energy. Therapy management engine 114 may process the historical exercise related data and determine that AMPK is activated by determining total volume of exercise completed over a period of time (e.g., one week). Total volume of exercise may be calculated using the duration and intensity of the exercise, where intensity may be a function of wattage, speed, incline, heart rate, lactate levels, etc. When the total volume of exercise is calculated in future exercise time periods is equal to or greater than the total volume of exercise in one or more past exercise time periods, the therapy management engine 114 may determine that AMPK was activated by the host.

In certain embodiments, therapy management engine 114 may determine that AMPK is activated based on the host's glucose levels. For example, if the host's glucose levels demonstrate the host has not had a meal in six hours and has completed an exercise session, therapy management engine 114 may determine the host's AMPK is activated. In another example, if the host's glucose levels demonstrate that the host has not consumed food in 24 hours, therapy management engine 114 may determine the host's AMPK is activated. In certain embodiments, if the host's glucose levels have remained low (e.g., demonstrating that the host has not consumed food) in three hours, therapy management engine 114 may instruct the host to continue fasting until at least 6 hours and/or to complete an exercise session to activate AMPK.

In certain embodiments, the host's historical exercise data may include physiological data associated with the host's previous exercise sessions, including starting lactate level at the outset of exercise, change in lactate levels, lactate rate of change during exercise and the host's at-rest baseline lactate level. Physiological data associated with the host's previous exercise sessions may also include other types of analyte data (e.g., glucose data, potassium data, etc.) and non-analyte data (e.g., heart rate data, temperature data, etc.) that may be used for host classifications.

In certain embodiments, additional analyte and/or non-analyte data may be utilized in classifying a host. For example, therapy management engine 114 may monitor a host's cortisol levels, sleep data (e.g., total sleep time, sleep efficiency, sleep latency, wake after sleep onset, number of awakenings, apneas and hypopneas, body movements, rapid eye movement (REM) sleep, non-REM sleep, snoring, Polysomnography (PSG), actigraphy, subjective sleep quality, sleep environment factors, glucose levels, respiratory rate, etc.), oxygen saturation at rest, heart rate, heart rate variability, etc. to determine the host's stress level, exercise readiness, etc. The host's exercise readiness may assist therapy management engine 114 in determining the host's classification. Additionally, the host's stress level may alter a host's metabolic adaptations, such as by affecting the host's insulin effect and/or glucose release and metabolism.

In certain embodiments, a host's genetic profile information may be received as input to classify a host. For example, therapy management engine 114 may be provided epigenetic metrics and/or changes in epigenetic metrics over time, such as the host's DNA methylation and/or mi-RNA expression. Epigenetic metrics may change as a host completes more exercise sessions and/or is subject to a training regimen. A host's genetic information may assist therapy management engine 114 in classifying the host.

At block 404, therapy management engine 114 may determine whether the host can be classified as a metabolically unfit host, a healthy host, or an athlete, based on the received inputs. As an example, therapy management engine 114 may determine that the host cannot be classified if certain types of inputs, such as historical exercise data, including lactate metrics associated with historical exercise sessions, are not available, in which case therapy management engine 114 proceeds to block 406. However, if the host is classifiable, therapy management engine 114 may proceed to classify the host based on the inputs received at block 402.

At block 405, therapy management engine 114 classifies the host (e.g., using a rules-based or AI/ML model) based on the inputs received at block 402. In embodiments where a rules-based model is used, the host's inputs may be mapped to a certain classification using, for example, a rules library. For example, the rules-based model may take inputs received at block 402 and classify the host into a metabolically unfit at block 410, average healthy host at block 412, or an athlete at block 414.

An example of a rule may include classifying the host as a metabolically unfit host if the inputs indicate that the host self-identified as a metabolically unfit host. Another example rule may include classifying the host as a metabolically unfit host if the inputs indicate that the host suffers from diabetes or liver disease. Another example rule may include classifying the host as a metabolically unfit host if the inputs include past exercise data showing lactate metrics that map to lactate metrics of metabolically unfit hosts, as described in more detail in relation to blocks 408 and 422. In certain embodiments, the rules may be more granular, such that a combination of rules and a variety of inputs to output a classification. As example of such a rule may be to classify the host as a metabolically unfit host, if the host self-identified as a metabolically unfit host, has liver disease, and/or their exercise data reflect lactate metrics that are indicative of a metabolically unfit host. If the host is classified as a metabolically unfit host, then therapy management engine 114 proceeds to block 416 to optimize exercise for the host based on the embodiments described in relation to FIG. 5A. Note that the embodiments described herein focus on optimizing exercise for a metabolically unfit host and that optimizing exercise for an average healthy host and an athlete is outside the scope of this disclosure.

Similarly, the host may be instead classified as a healthy host or an athlete using the rules-based model described above. For example, lactate metrics indicated by the historical exercise data of the host may be used, as described in relation to blocks 408, 424, and 426, to classify the host as a healthy or an athlete.

In certain embodiments, instead of a rules-based model, an AI/ML (interchangeably referred to as an “ML model” for simplicity) model may be used to predict the host's classification. For example, some of all of the inputs received at block 404 may be used as input into a model that is trained to classify a corresponding host. In such cases, the model is trained using a training dataset, including historical population-based data of many hosts, who have been already classified into metabolically unfit, healthy, or athlete hosts. In such an example, the training dataset is labeled with such classifications. In certain embodiments, the output of the model may be accompanied by a confidence score. In cases where the confidence score is below a certain threshold, therapy management engine 114 may determine that the host is not classifiable and proceed to block 406.

At block 406, if the host is not classified based on the inputs at block 402, the host may be guided through a trial exercise session to monitor lactate measurements and determine the host classification based on the host's lactate response to exercise. For example, therapy management engine 114 may suggest the host complete an exercise session for a specific duration (e.g., 15 minutes) at a gradually increasing intensity (e.g., increasing speed, wattage, heart rate, incline, etc. by a specific metric each minute). In embodiments where an exercise machine is used, therapy management engine 114 may automatically instruct the exercise machine 108 to begin a trial exercise session according to the gradually increasing intensity described above. The intensity may increase at the same rate for all hosts completing a trial exercise session in order to accurately compare the host's lactate response to the lactate response of other hosts. Therapy management engine 114 may then monitor the host's lactate response to the exercise.

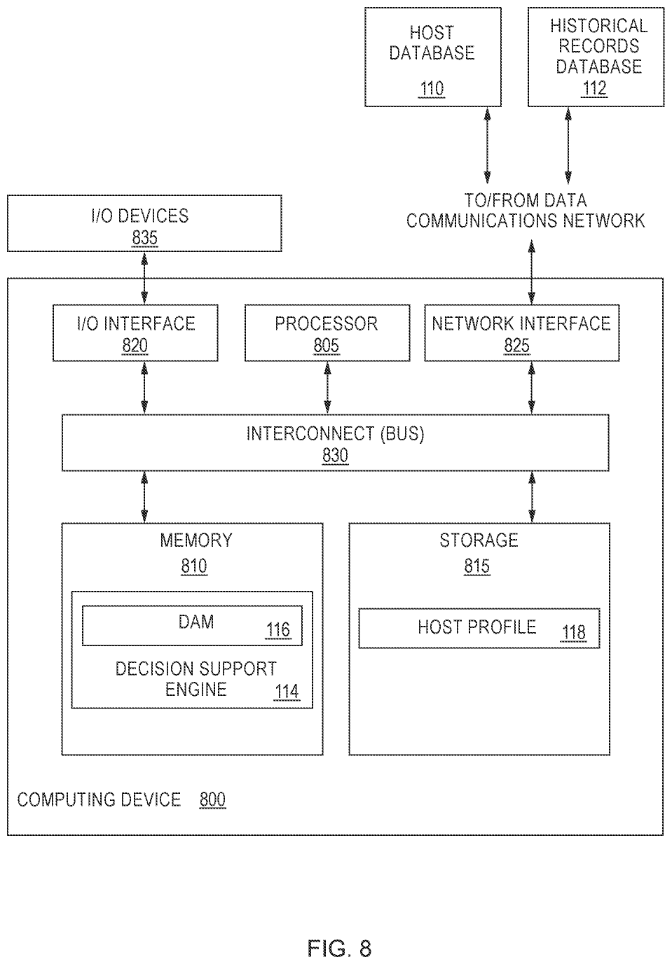

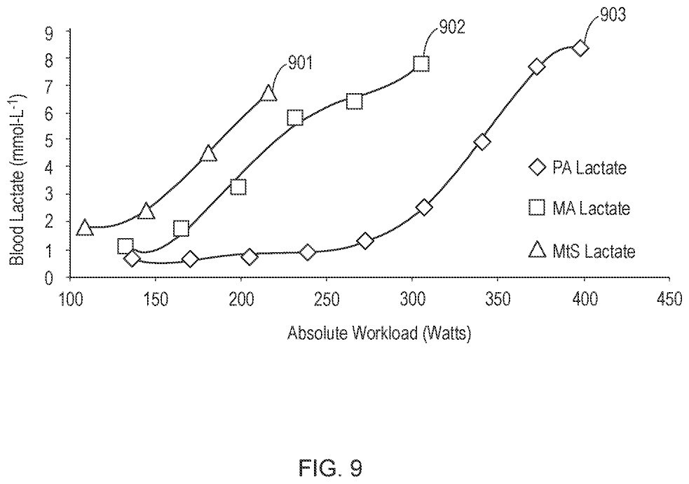

At block 408, therapy management engine 114 may be configured to classify the host into a metabolically unfit host at block 422, average healthy host at block 424, or an athlete at block 426. A host may be classified utilizing different lactate metrics obtained from the trial exercise session, including starting lactate level at the outset of exercise, the rate of change of lactate throughout the trial exercise session, the presence of a lactate “trough” (as discussed in FIG. 9) and the intensity/time at which the host reaches their lactate threshold. Other information, such as the inputs received at block 402 may also be used for classifying the host, as described below.

In certain embodiments, therapy management engine 114 may use a rules-based model to classify the host. Such a rules-based model may include rules that are defined based on the lactate metrics, obtained during the trial exercise session, a described above. For example, such rules may define ranges, based on empirical research involving population-based data, for lactate levels at the outset of exercise, the rate of change of lactate throughout the trial exercise session, the presence of a lactate “trough,” and the intensity/time at which the host reaches their lactate threshold, etc. In certain embodiments, classification of the host may include providing the host with a score based on the host's fitness within the classification. For example, a host classified as an metabolically unfit host with a goal to improve general metabolic fitness may receive a score (e.g., a score between 1-10, 10 being optimal) in addition to the classification to further monitor the host's improvement in metabolic fitness over time. In another embodiment, a host classified as an metabolically unfit host with a goal of improving strength and building muscle mass may receive a score based on the host's specific goal.

As an example of such rules, if a host begins a trial exercise session and the lactate level at the outset of exercise is 3-4 millimolar, and the rate of change of lactate is negative (as skeletal muscles begin to metabolize excess blood lactate at the start of exercise), therapy management engine 114 may deduce that the host is a metabolically unfit host, although may continue the exercise session to monitor for other lactate patterns. As the host continues to increase intensity during the trial exercise, the host may experience a lactate “trough” where the host's lactate has a negative rate of change that is beginning to approach zero. Once the rate of change is zero, as intensity continues to increase, the lactate rate of change may remain constant at zero for a time prior to beginning to increase with a positive lactate rate of change. The lactate “trough” where the rate of change of lactate approach zero during an exercise session following a negative lactate rate of change and prior to a positive rate of change may be indicative of a metabolically unfit host.

In certain embodiments, the classification ranges may be based on intensity at which the host reaches the lactate threshold or a maximal lactate steady state, among other considerations. As such, therapy management engine 114 may consider the intensity and/or time since the start of exercise at which the host reaches their lactate threshold (e.g., the lactate rate of change becomes exponentially positive following the lactate “trough”). Therapy management engine 114 may then classify the host as a metabolically unfit host depending on the intensity and the amount of time it took for the host to reach their lactate threshold. A host may then be classified as a metabolically unfit host if, for example, the host's lactate threshold occurs below an intensity of 125 watts. For instance, a metabolically unfit host may have a lactate threshold at 100 watts of intensity, or a speed of 5 miles per hour on a treadmill.

In addition to the rules described above, which are based on the lactate metrics obtained during a trial exercise session, the rules-based model may further include other rules, based on any of the information provided as part of host profile 118 and/or any information provided through host input as described above.

In certain embodiments, instead of or in addition to a rules-based model, an ML model may be used that takes the lactate metrics obtained for the host during the trial exercise session and/or other inputs received at block 402 to classify the host. In certain embodiments, in addition to the lactate metrics and inputs, the model may also receive environmental data as input to classify the host. Environmental data may include altitude at the time of the trial exercise session, as a higher altitude causes a decrease in available oxygen and, therefore, the host may produce more lactate during exercise. For example, some of the lactate metrics obtained during the trial exercise session, environmental data, and/or the inputs received at block 404 may be used as input into a model that is trained to classify a corresponding host. In such cases, the model is trained using a training dataset, including historical population-based data of many hosts, who have been already classified into metabolically unfit, healthy, or athlete hosts based at least on their corresponding lactate metrics. In such an example, the training dataset is labeled with such classifications.

In certain embodiments, the host may be classified as either an average healthy host or an athlete. For example, a host may begin a trial exercise session and the lactate level at the start of exercise may be about 1 millimolar to 1.5 millimolar. Further, in the first few minutes of the exercise session, the lactate rate of change may be slightly negative or positive, in which case therapy management engine 114 may deduce that such lactate metrics do not map to a metabolically unfit host. As such, the host may not be classified as a metabolically unfit host and therapy management engine 114 may continue monitoring the trial exercise session. As the host continues to increase intensity during the trial exercise, the host may experience a gradual increase in lactate, as the lactate rate of change begin to approach zero. Subsequently, the lactate rate of change may remain constant for a time period, prior to reaching the host reaching their lactate threshold, where lactate slope and rate of change begin to increase exponentially. The intensity and/or time from starting exercise at which the host's lactate threshold occurs may then be used in determining the classification of the host. For example, a host may be classified as a healthy host if the host's lactate threshold occurs between an intensity of 125 watts to 250 watts. Finally, a host may be classified as an athlete if the host's lactate threshold occurs at an intensity greater than 250 watts.

In certain embodiments, a host may have past exercise data, including past trial exercise session data and past classifications, stored in historical records database 112, though the host may not have completed an exercise session in an extended time period (e.g., 1-2 weeks). Therapy management engine 114 may consider the consistency of exercise when classifying or reclassifying a host. For example, a host may be classified as a healthy host based on past classifications and/or a series of exercise sessions. However, after a length of time when the host does not exercise, therapy management engine 114 may reclassify the host to determine whether the host would still be classified as a healthy host following a length of time without exercise.

FIGS. 5A-5C describe an example method for optimizing exercise sessions and providing feedback to a metabolically unfit host regarding metabolic fitness, post-exercise nutrition or glucose administration, and effectiveness of exercise sessions. As an example, after the host is classified as a metabolically unfit host, therapy management engine 114 may be configured to guide the host through a series of exercise sessions to help with improving the host's metabolic fitness. For example, the host may use application 106 or 111 to indicate that their goal is to improve their metabolic fitness and to request guidance for an exercise session (e.g., the first of a series of exercise sessions). In such an example, application 106 or 111 may then present a user interface for the host to select whether they would like to perform Zone 2, resistance training, or HIIT for their upcoming exercise session.

Method 500 begins at block 501, where therapy management engine 114 determines whether the host has sarcopenia. Sarcopenia is the age-related loss of skeletal muscle mass and strength. Therapy management engine 114 may determine the host has sarcopenia based on the host's body composition information (e.g., from a CT scan, MRI scan, and/or DEXA scan), creatinine data, and/or creatinine to cystatin C ratio as described herein. At block 502, therapy management engine 114 determines whether the host has selected a HIIT, resistance training, or Zone 2 exercise type. If the host has selected Zone 2, therapy management engine 114 will proceed directly to block 504. If the host has selected HIIT, therapy management engine 114 will proceed to the method described in relation to FIG. 5B. If the host has selected resistance training, therapy management engine 114 will proceed to the method described in relation to FIG. 5C.

If the host has not selected or denoted a type of exercise, at block 522, therapy management engine 114 may suggest Zone 2 for hosts with high baseline lactate (e.g., greater than 2 millimolar) due to liver disease, as Zone 2 would support fat oxidation and initially help the body consume lactate that the liver is not clearing. Generally, Zone 2 would be expected to be most effective for improving liver health. However, if the host's baseline lactate levels are normal, therapy management engine 114 may suggest an exercise type based on the host's goals. In certain embodiments, therapy management engine 114 may suggest HIIT or resistance training if the host is under a time constraint. In certain embodiments, therapy management engine 114 may suggest mostly resistance training, combined with some Zone 2, for a host with a goal to build more muscle, or a host determined to have sarcopenia. In certain embodiments, therapy management engine 114 may suggest resistance training if the host's goal is to build muscle mass and/or increase resting metabolic rate. In certain embodiments, based on the medical history of the host, such as medical history stored in host profile 118, therapy management engine 114 may recommend an exercise session that is unlikely to cause regression of a current injury. For example, if a host reported having ankle surgery, therapy management engine 114 may recommend a low impact Zone 2 exercise or an upper body focused resistance training exercise session. Additionally, if the host has a prescribed physical therapy regimen based on a recent surgery or injury, therapy management engine 114 may recommend the host incorporate the physical therapy regimen into their recommended exercise session.

Further, therapy management engine 114 may determine whether to recommend HIIT, resistance training, or Zone 2 exercise based on the host's lactate level at the outset of exercise. For example, if a host normally has a lactate level of 1.8 millimolar at the outset of exercise but on this day the host's lactate level is 3 millimolar at the outset of exercise, therapy management engine 114 may suggest the host complete a Zone 2 exercise session (instead of a HIIT exercise session) to reduce lactate levels.

In other embodiments, therapy management engine 114 may suggest HIIT or resistance training for improving resting metabolic rate over time, or when the host may have limited time to complete an exercise session. In certain embodiments, therapy management engine 114 may suggest a mix of one or more types of exercise based on the host's demographics and/or goals. For example, if a metabolically unfit host is determined to be sarcopenic at block 501, therapy management engine may suggest the host completes 75-80% resistance training to counteract muscle degradation and 20-25% Zone 2 exercise. In certain embodiments, a percentage may be measured based on time, calorie and/or analyte expenditure, metabolic equivalent, etc. Alternatively, if a metabolically unfit host is determined to not suffer from sarcopenia at block 501, therapy management engine 114 may suggest the host completes 20-25% resistance training and 75-80% Zone 2 exercise. If the host, at block 502 selects Zone 2 or therapy management engine 114 suggests Zone 2 to the host at block 522, then therapy management engine 114 proceeds to block 504.

At block 504, therapy management engine 114 may determine whether the host has completed a Zone 2 exercise session in the past, for which data is available, by referencing the host's profile 118. For example, therapy management engine 114 may examine the exercise data in the host profile 118 to determine if there is data relating to one or more past Zone 2 exercise sessions of the host.

At block 506, if the host has completed a Zone 2 exercise session in the past for which data is available, therapy management engine 114 determines personalized exercise guidance for the host based on the host's own historical exercise data. For example, exercise data from the host profile 118 may be used to determine a set of exercise parameters for the host's exercise session. The set of parameters may include exercise intensity (e.g., at the outset of the session and throughout), exercise duration, etc. Exercise intensity itself can be a function of speed, resistance, incline, etc. Providing personalized guidance based on the host's own historical exercise data may be accomplished in a variety of ways. For example, the host's own historical exercise data may be used in conjunction with a rules-based model to provide the set of exercise parameters for the host's exercise session. Additionally or alternatively, one or more ML models, trained based on the host's own historical data, may be used to provide the set of exercise parameters for the host's exercise session. For example, training server system 140 may retrieve exercise data related to the specific host from the host profile 118 to train one or more ML models.

At block 508, if the host has not previously completed a Zone 2 exercise session for which data is available, therapy management engine 114 may use population-based models (non-personalized) to determine exercise parameters for the host, at least until the host performs one or more exercise sessions and personal exercise data is available for the host. For example, therapy management engine 114 may use a rules-based model that may define Zone 2 exercise parameters defined based on empirical research involving population data. For example, the Zone 2 exercise parameters may be determined using rules based on parameters that have been effective for a host population similar (e.g., based on demographic variables (e.g., age, gender, weight, height, BMI, etc.)) to the host to reach the Zone 2 lactate range.

Additionally or alternatively, therapy management engine 114 may use one or more ML models to provide exercise parameters to the host to reach the Zone 2 lactate range. For example, an ML model may be trained based on population-based training data associated with metabolically unfit hosts who reached the Zone 2 lactate range, the dataset including data records each including exercise parameters, analyte data, non-analyte data, and/or other relevant information in the corresponding host's profile 118 for each exercise session. As an example, each data record in the training dataset may include time-stamped exercise parameters along with corresponding time-stamped analyte data and non-analyte data for the host's exercise sessions. The data records may be labeled with one or more exercise parameters. Using such a training dataset, the model can be trained to predict one or more exercise parameters to help the host reach the Zone 2 lactate range.

Prior to guiding the host through an exercise session based on the exercise parameters determined at blocks 506 and 508 using personalized or population-based models, therapy management engine 114 may instruct the host to begin exercise at a warm up intensity for a duration. In certain embodiments, the intensity may be one-half of the intensity later provided to the host to reach the Zone 2 lactate range. In certain embodiments, the duration of the warm up intensity may be about 5 minutes or 10 minutes.

At block 510, once the warm up duration is complete, therapy management engine 114 may instruct the host or an exercise machine to increase intensity to the exercise parameters determined in block 506 or 508.

In embodiments where the host is completing the exercise session on an exercise machine (e.g., exercise machine 108), the therapy management engine 114 may automatically set or gradually increase the intensity (e.g., speed, incline, and/or resistance, etc.). Therapy management engine 114 may automatically set the exercise machine to the exercise parameters determined at blocks 506 and 508. In embodiments where the host is not using an exercise machine, therapy management engine 114 may instruct the host to increase the intensity of their exercise by gradually increasing various exercise parameters until the optimal Zone 2 lactate range is achieved. Increasing intensity may be accomplished by altering various exercise parameters associated with an exercise machine (e.g., exercise machine 108) or expending additional effort (in the form of speed or wattage) in cases where the host is not using an exercise machine.

Typically, metabolically unfit hosts may have a starting lactate level greater than 1.8 millimolar at the outset of exercise. In some cases, a metabolically unfit host may have a starting lactate levels of 4 millimolar at the outset of exercise. Then, unlike a healthy host, an initial drop in lactate to reach a lactate “trough” (e.g., a negative rate of change of lactate) may be expected for metabolically unfit hosts, as the skeletal muscles begin to clear lactate from the body. Following such reduction in lactate levels, the lactate rate of change may begin to approach zero at around 2 millimolar, representing the optimal Zone 2 lactate range. As such, therapy management engine 114 is configured to continuously monitor the host's physiological parameters to determine the optimal exercise intensity that would allow the host to reach and remain in the lactate range where rate of change is at or approaching zero. For example, if the host does not exercise with enough intensity, the host will not reach the optimal Zone 2 lactate range. Alternatively, if the host exercises with too much intensity, the host will exceed the Zone 2 lactate range quickly, and continue to experience an increase in lactate beyond the desired range. Therefore, it is critical for therapy management engine 114 to continuously monitor the host's lactate levels, including lactate rate of change, and physiological parameters (e.g., heart rate, respiratory rate, glucose metrics, wattage, speed, power, accelerometer data, etc.) to instruct the host to increase or decrease intensity to remain within the optimal Zone 2 lactate range, as further described in relation to blocks 512 and 514.

At block 512, therapy management engine 114 may determine if the host is already within the Zone 2 lactate range or trending to reach the expected Zone 2 lactate range, for example, based on the host's lactate metrics. If the host is already within range or trending to reach Zone 2 lactate range (e.g., lactate rate of change approaching zero), therapy management engine 114 may instruct the host to maintain the current intensity.

Note that the expected Zone 2 lactate range, also referred to as defined range herein, may be between 1.9 to 2.1 millimolar, between 1.7 to 2.3 millimolar, or between 1.5 to 2.5 millimolar, for example. However, the range may be different for different hosts and therefore, therapy management engine 114 is configured to observe lactate levels and determine whether the host is approaching a lactate rate of change of or near zero, which demonstrates the host is or will be in a Zone 2 steady state lactate range. In order to determine whether the host is approaching a lactate rate of change of or near zero, therapy management engine 114 may use one of a variety of rules-based or ML models to take the host's lactate levels and/or other lactate metrics (e.g., rate of change), as well as other physiological parameters to project whether the host is soon approaching rate of change of or near zero.

In certain embodiments, therapy management engine 114 may determine whether the host has reached the Zone 2 range based on potassium metrics. For example, based on the host's historical data, therapy management engine 114 may determine an individualized potassium level range that corresponds to the host's Zone 2 steady state lactate range. During future exercise sessions, therapy management engine 114 may determine the host is in the Zone 2 lactate range based on whether the host's potassium levels are within the Zone 2 individualized potassium range.

In certain embodiments, as described above, determining whether the host has reached a certain metabolic state (e.g., Zone 2) is accomplished primarily by monitoring the host's lactate metrics. However, a host's lactate metrics may, in some cases, be unavailable and/or lactate measurements received from a continuous lactate sensor may be delayed in relation to real-time lactate blood measurements. To address the potential unavailability or the lag, in certain embodiments, therapy management engine 114 may additionally or alternatively use some of the host's physiological parameters (e.g., heart rate, respiratory rate, glucose metrics, wattage, speed, power, accelerometer data, etc.) as surrogates for lactate data. Such physiological parameters may have a direct correlation with lactate metrics and therefore may be used to estimate real-time lactate metrics during an exercise session, when such lactate metrics are not available. As such, during a first exercise session, where a continuous lactate sensor is available, lactate measurements may be obtained and correlated to the host's corresponding physiological parameters. Those physiological parameters can then be used during future exercise sessions, where lactate data is not available, as surrogates for lactate metrics that are indicative or certain metabolic states (e.g., Zone 2, resistance training, or HIIT).

At block 514, therapy management engine 114 may observe lactate metrics (e.g., lactate values and trends), and/or the host's physiological parameters and, where the host is not trending to achieve the optimal Zone 2 lactate range, therapy management engine 114 may instruct the host to adjust exercise intensity. For example, the host is considered not to be trending to achieve the desired lactate range when the host is either not exercising with enough intensity to achieve the Zone 2 lactate range (e.g., 1.9-2.1 millimolar), or the host is exercising with too much intensity such that the host's lactate levels will reach the Zone 2 lactate range but then increase beyond the desired range.

A variety of models (e.g., rules-based, machine learning or predictive algorithms) may be used to determine the optimal exercise parameters and, therefore, the ideal intensity to ensure that the host's lactate level reaches the optimal Zone 2 lactate range but does not increase beyond the Zone 2 lactate range. In certain embodiments, a rules-based model may be utilized where various rules can be defined around a set of parameters, including the host's current lactate level, the current lactate rate of change, time since outset of exercise, intensity of exercise, as well as other physiological parameters (e.g., heart rate, respiratory rate, glucose metrics, wattage, speed, power, accelerometer data, etc.). For example, one example rule may dictate that if the host's current lactate level is X, the current lactate rate of change is Y, and the time since the outset of exercise is Z, then the intensity should be Q. Q may be a set of exercise parameters that would bring the host into the Zone 2 range such as speed, resistance level, elevation, wattage, etc. The rules may get much more granular and involve a whole host of other physiological and demographic metrics. For example, a rule may say if the host is above the age of 60, has a heart rate of A, temperature of B, current lactate level of X, current lactate rate of change of Y, time since the outset of exercise of Z, then the intensity should be Q.

In certain other embodiments, one or more predictive algorithms or models may be used to predict the optimal intensity to achieve the desired Zone 2 lactate range. For example, one or more models that may be the same or different from the personalized or population-based models described in relation to blocks 506 and 508 may continuously run to take a set of inputs (e.g., host's current lactate level, the current lactate rate of change, time since outset of exercise, as well as other inputs received at block 402), and/or other physiological parameters (e.g., heart rate, respiratory rate, glucose metrics, wattage, speed, power, accelerometer data, etc.) and output the optimal intensity and/or corresponding exercise parameters for achieving the optimal Zone 2 lactate range.

When instructing the host to increase or decrease intensity, therapy management engine 114 may examine whether the host is exercising outdoors. For example, non-analyte sensor data from a temperature sensor may suggest that the host is exercising outdoors or the host selecting, for example, outdoor run or walk as exercise type may be used by therapy management engine 114 to determine the host is exercising outdoors. When the host is exercising outdoors, therapy management engine 114 may consider air temperature, air quality, humidity, wind speed, known allergens, and elevation when suggesting an increase or decrease in intensity.

For example, based on sensor data from temperature sensors, therapy management engine 114 may notify the host that the environment is too hot and it is not safe to exercise at the usual intensity (e.g., recommend the exercise intensity as a function of temperature to reach Zone 2 lactate range, even if the recommended intensity is lower/higher than historic data), or that the host should stop exercising.

Further, therapy management engine 114 may instruct the host to decrease intensity or stop exercising based on the correlation between potassium levels and temperature sensor data at higher outdoor temperatures. For example, where the host may perceive that they are at the correct intensity at high temperatures, potassium levels may increase beyond expected levels due to the high temperatures. Where the host perceives low to medium exertion but potassium sensors demonstrate increased potassium levels, the host may be at risk for a cardiac event (e.g., arrhythmia, cardiac arrest, etc.). Therefore, therapy management engine 114 may instruct the host to decrease intensity or stop exercising at high temperatures when the host's potassium levels increase beyond expected levels.

Further, therapy management engine 114 may use the correlation between lactate levels at specific intensity levels and temperature sensor data to provide more accurate intensity instructions to the host. For example, lactate levels may be higher in higher temperatures at the same intensity. Therefore, at high temperatures, therapy management engine 114 may instruct the host to exercise at a lower intensity to achieve the Zone 2 lactate range compared to the intensity required under typical temperature conditions. Conversely, at lower temperatures, the lactate response may be lower, requiring an increase in intensity to reach the optimal Zone 2 lactate range. Without considering temperature sensor data, at high temperatures, therapy management engine 114 may instruct the host to increase intensity which may cause the host to increase lactate beyond the optimal Zone 2 lactate range, and cause heat stroke or other safety concerns. At colder temperatures, therapy management engine 114 may not account for the higher intensity required to reach the Zone 2 lactate range when compared to typical temperatures causing a less effective exercise session.

In addition to temperature, when the host is exercising outdoors, therapy management engine 114 may also take into account elevation when providing intensity instructions. For example, if the host is jogging outdoors and is approaching an upcoming hill, therapy management engine 114 may instruct the host to maintain or even decrease the intensity. Not doing so may cause the host to exercise with too large of an increase in intensity when combined with the hill. This intensity may cause the host's lactate to overshoot the optimal Zone 2 lactate range. Therefore, therapy management engine 114 may take into account the change in elevation or expected change in elevation based on GPS and/or map data and adjust intensity recommendations accordingly.

Further, when the host is exercising outdoors, they may complete an exercise session (e.g., run, walk, bike, etc.) on a specific route one or more times. In such cases, therapy management engine 114 may recognize the route based on GPS data and past exercise data, and provide certain locations where the host should increase or decrease intensity in order to maintain Zone 2 lactate range.

Further, whether the host is exercising indoors or outdoors, therapy management engine 114 may determine whether the host is demonstrating signs of cardiac stress based on the host's cardiac metrics (e.g., through a heart rate monitor or an ECG). For example, therapy management engine 114 may monitor various cardiac metrics for signs of atrial flutter, tachycardia, atrial fibrillation, high heart rate, etc. If the host is experiencing abnormal cardiac metrics in combination with high lactate as a result of exercise, therapy management engine 114 may instruct the host to decrease intensity and/or stop exercising and seek medical attention for potential cardiac complications.

Following a suggested increase or decrease in intensity at block 514, therapy management engine 114 may return to block 512 to determine whether the host is within the Zone 2 lactate range or whether the host's lactate levels and lactate rate of change demonstrate that the host is trending to reach lactate levels within the Zone 2 lactate range. If not, therapy management engine 114 returns to block 514 to adjust intensity accordingly. If yes, therapy management engine 114 will proceed to block 516.

At block 516, therapy management engine 114 may instruct the host to maintain current intensity for a specified duration (e.g., 5 minutes or 10 minutes). The specified duration may be the ideal length of a Zone 2 exercise for the host's goals. The specified duration may also be based on the most effective length of exercise for the host based on past exercise data or the most effective length for historical hosts with similar demographics to the host.

At block 518, once the duration is complete, for non-diabetic hosts, therapy management engine 114 may guide the host to complete a cool down at a lower intensity (e.g., walking) and monitor lactate levels (or surrogate parameters) to ensure that the host's lactate levels have returned to baseline levels (e.g., 1.8 millimolar). An active cool down allows the body to clear excess lactate as a result of exercise and optimize fat burning post-exercise.

For hosts with Type 1 diabetes, therapy management engine 114 may recommend the host complete a cool down at a lower intensity (e.g., walking), and/or consume specific post-exercise nutrition and/or administer glucose to prevent immediate or possible hypoglycemia. For a host with Type 2 diabetes, therapy management engine 114 may also recommend that the host avoids administering insulin. In particular, aerobic exercise, such as Zone 2, lowers blood glucose levels. If the host is experiencing low blood glucose after an exercise session, therapy management engine 114 may determine whether the host's glucose levels are dangerously low or expected to become dangerously low (e.g., less than 70 mg/dL). If following a HIIT session, the host's glucose is low but not requiring immediate action, then consuming glucose or administering insulin would not be advised as the host will experience a glucose spike once the host's liver and skeletal muscles have cleared the excess lactate during active cool down. If therapy management engine 114 determines the host's glucose levels are dangerously low or expected to become dangerously low, therapy management engine 114 may hosthostinstruct the host to consume a small amount of food to increase their glucose levels following a Zone 2 exercise session to avoid hypoglycemia. A meal or drink that is high in sugar (e.g., Gatorade) may not be recommended as it would also spike lactate levels, which inhibits the cool down process, decreases fat oxidation, and decreases overall exercise effectiveness. If the host experiences an increase in lactate levels following an exercise session, the glucose levels of the host may be expected to increase as lactate levels decrease. In this example, the host may be instructed to avoid consuming food containing large amounts of glucose, and instead consume foods containing small amounts of glucose while monitoring glucose levels to avoid hyperglycemia.

Further at block 518, therapy management engine 114 may provide feedback to the host on metabolic fitness, and effectiveness of the exercise session as well as recommendations for future exercise sessions, diet recommendations, liver health status, and estimated fat oxidation and weight loss.

For example, feedback associated with metabolic fitness (or improvement therein) may be provided to the host at block 518. Metabolic fitness may be determined by the host's ability to reach the Zone 2 lactate range with a similar intensity in future exercise sessions. For example, if the host required a certain speed or wattage to reach the Zone 2 lactate range in past exercise sessions, therapy management engine 114 may monitor whether this intensity is effective to reach the Zone 2 lactate range in future exercise sessions. Over time, if the host requires greater intensity (e.g., speed or wattage) to reach the Zone 2 lactate range, the host may have increased metabolic fitness, as the host's liver and skeletal muscles are becoming more efficient at clearing lactate from the body at greater intensity.

In certain embodiments, feedback provided to the host at block 518 may include a reward and/or gamification feature. The host may receive various badges, stars and/or other rewards when the host progresses closer to their goal (e.g., a certain number of exercise sessions, a certain improvement in metabolic fitness, etc.). In certain embodiments, feedback provided to the host at display device 107, for example, may be configured to connect to the host's social media account where the host can share updates and rewards via social media accounts with followers, family, and friends. Allowing the host to share with others via social media may provide support and encouragement to the host and encourage the host to continue their exercise regimen.

Additionally, feedback associated with metabolic fitness may be based on changes in a host's lactate response to exercise over time. For example, as the host completes exercise sessions, therapy management engine 114 may provide feedback on an improvement in lactate clearance rate over time, which demonstrates improved metabolic fitness. Feedback on improved metabolic fitness may also be based on the lactate clearance over time relative to whether the host completed a cool down following the exercise session, as a cool down encourages lactate clearance. Therapy management engine 114 may determine the intensity and duration of the cool down when monitoring the host's lactate clearance rate over time. In certain embodiments, therapy management engine 114 may instruct the host to consume a specific amount of lactate (e.g., oral lactate tolerance test) while not exercising to determine lactate clearance rate and improvement in lactate clearance rate in the absence of muscle consumption of lactate during exercise. An improvement in lactate clearance rate in response to the same amount of lactate and in the absence of exercise would indicate an improvement in the host's metabolic fitness.

In certain embodiments, feedback on effectiveness of the exercise session may include monitoring caloric burn and overall energy expenditure following exercise. In certain embodiments, caloric burn and overall energy expenditure may be determined using the metabolic equivalent of task (MET) equation or the Harris-Benedict equation.

Additionally, therapy management engine 114 may provide diet recommendations tailored to specific hosts based on the host's lactate response to Zone 2 exercise. For example, diet recommendations may include specific macronutrients to consume or timing of meals relative to the exercise session. For example, following an exercise session, a host may be recommended to consume protein within a specified time period (e.g., within 20 minutes of exercise) in order to build muscle mass. In another example, the host may be instructed to not consume, or limit consumption of lactate, glucose or fructose within a specified time period (e.g., 2 hours), in order to maintain higher fat oxidation levels.

Further, therapy management engine 114 may provide feedback associated with the host's liver health. Liver health feedback may include providing the host information about a liver disease diagnosis, and/or liver disease improvement/deterioration. Information about liver disease improvement/deterioration can obtained based on monitoring for liver disease progression using lactate measurements pre- or post-exercise (e.g., fasting lactate, overnight baseline lactate, lactate clearance rates post-exercise, lactate levels during exercise), other analyte levels (e.g., liver lipid content, liver enzyme status, glycogen storage status, bilirubin levels, or insulin resistance) and liver health testing (e.g., determining liver fat through MRI-PDFF, determining liver inflammation through ALT/AST levels, determining fibrogenic activity through Pro-C3 tests, determining blood clotting time through international normalized ratio test or detecting liver elasticity through MRE imaging).

Additionally, therapy management engine 114 may project fat oxidation at rest, post-exercise and provide the host information about the projected fat oxidation. Therapy management engine 114 may consider host input (e.g., host's diet choices, sleep, medical history, or medications taken) and past exercise data (e.g., length of exercise) to determine metabolic response to exercise and fat oxidation. Metabolic response may be the host's ability to clear lactate and glucose from the body post-exercise when compared to past exercise. Fat oxidation may be determined based on the portion of carbohydrate and lipid levels that account for the total calorie burn (e.g., using indirect calorimetry). The lipid level portion of the total calorie burn may demonstrate the host's fat oxidation as a result of exercise.

In certain embodiments, therapy management engine 114 may correlate lower lactate levels during later exercise sessions as compared to previous exercise sessions with greater fat oxidation. Because blood lactate accumulation may be negatively correlated with fat oxidation, lower lactate accumulation as a result of exercise and/or improved lactate clearance rates would result in greater expected fat oxidation. For example, a host may experience maximal fat oxidation between 1.8 and 2.1 millimolar lactate. Therefore, if a host's baseline lactate is above 2.1 millimolar, then a reduction in lactate levels during may be observed and the intensity level should be increased to bring lactate levels back up to about 2.1 mM to achieve maximal fat oxidation. Additionally, following an exercise session, a lower baseline lactate level may be indicative of improved fat oxidation for the host at rest. For example, if a host has a baseline lactate level of 3 millimolar at the outset of a first exercise session and, as a result of diet and/or exercise guidance the host's baseline lactate level improves to 2 millimolar, therapy management engine 114 may determine the host has accomplished improved fat oxidation.

Therapy management engine 114 may also measure metabolic rate of a host post-exercise and provide the host with their metabolic rate or improvement in metabolic rate. Therapy management engine 114 may use peak lactate, lactate clearance rate, and lactate rate of change to predict a metabolic rate post-exercise. Therapy management engine 114 may also use indirect calorimetry, the Weir equation, or various analyte measurements (e.g., glucose, free fatty acids, beta hydroxyl butyrate and glycerol) to measure a host's metabolic rate over a series of exercise sessions to determine an improvement in metabolic rate. Improvement in metabolic rate may reflect improved liver health, improved physical fitness, and weight loss over time.

Therapy management engine 114 may also be configured to interface with a continuous positive airway pressure (CPAP) machine which may function as an indirect calorimeter. If the host uses a CPAP machine on a daily basis, utilizing a CPAP machine as an indirect calorimeter may be beneficial to gather consistent indirect calorimetry readings. For example, the CPAP may be configured as an indirect calorimeter to measure metabolic usage of specific fuel (e.g., fat or carbohydrates) via a carbon dioxide (CO2) sensor and a flow sensor to determine the rate of CO2 production. In a CPAP machine, a CO2 sensor and a flow sensor may be added to the mask or hose of the CPAP to measure oxygen consumed from inhaled air and CO2 production from exhaled air while sleeping or at rest. The use of the CPAP machine to measure energy expenditures at night may be useful to compare with the host's metabolic rate and energy expenditures following exercise.

Therapy management engine 114 may also be configured to receive input from the host following an exercise session. For example, the host may be prompted to provide input to therapy management engine 114, via a user interface of display device 107, user interface 113 of exercise machine 108, etc. Therapy management engine 114 may request the host's perceived exertion level following a Zone 2 session. In certain embodiments, the host may be given choices to select the host's perceived exertion level during the exercise session (e.g., “I feel like I can keep going at this pace” or “I am reaching my limit” or “I am feeling nauseous and cannot continue at this intensity”). Receiving input from the host regarding the perceived exertion level may assist therapy management engine 114 in suggesting an increase or decrease in intensity in future exercise sessions. For example, if the host provides input that they can continue at this pace after an exercise, where the host did not reach the optimal Zone 2 lactate range until 20 minutes of exercise, therapy management engine 114 may suggest the host exercise at a higher intensity in future exercise sessions. The higher intensity would allow the host to reach the Zone 2 lactate range more quickly to maximize exercise effectiveness without causing overexertion.

However, in some cases, a host who is taking a beta blocker medication may have a higher perceived exertion level than reality. For example, a host on a beta blocker medication may believe they are overexerting and provide input to that effect, but the host's lactate measurements may signal to therapy management engine 114 that the host was in the Zone 2 lactate range during the perceived overexertion. In such an example, therapy management engine 114 may be less likely to alter intensity in future exercise sessions based on the host's perceived exertion level.

In some cases, potassium metrics may be utilized to determine when the host is suffering from overexertion during or after an exercise session. For example, the host may be suffering from overexertion where the host's potassium levels reach an absolute maximum potassium level, an individualized maximum potassium level, or exceed a set duration of time where potassium levels are above the maximum potassium level. In another example, the host may also be suffering from overexertion if the host's potassium levels show a high rate of change, which can demonstrate that the host's potassium levels will eventually exceed an absolute or individualized maximum potassium level.

At block 520, once the host has completed the Zone 2 exercise, therapy management engine 114 may utilize the information obtained about the host during the exercise session (e.g., analyte data, non-analyte data, host inputs, etc.) to optimize future exercise sessions. For example, the information may be used in one or more rules-based models at block 506 during a future exercise session. In another example, the ML models utilized at blocks 506 or 508 may be personalized (e.g., trained/retrained) using the information. As such, for future exercise sessions, therapy management engine 114 may be able to more effectively predict the optimal exercise parameters that may get the specific host to the Zone 2 lactate range.

As described above, optimizing exercise sessions for a host may be accomplished by monitoring the host's response to exercise and providing intensity guidance based on the host's lactate metrics obtained using a continuous analyte sensor system (e.g., sensor system 104). However, in some cases, lactate metrics may not be available and/or may experience a time lag (e.g., the lactate metrics may not be indicative of real-time blood lactate levels). In cases where lactate metrics are not available and/or experiencing a lag, certain physiological parameters that have a direct correlation with lactate metrics may be used as surrogates for lactate metrics. These surrogate parameters, herein referred to as physiological parameters, may act as an estimation of real-time lactate levels. Physiological parameters may include heart rate, respiratory rate, glucose, wattage, speed, power, data from accelerometer, etc. For example, in a prior exercise session, a host may be at 120 beats per minute heart rate at the time the host reaches Zone 2 lactate range (as determined by post-exercise analysis taking into account the lag in lactate measurements). During future exercise sessions, heart rate, or other real time physiological parameters may be used to estimate the host's real time lactate level, such that when the host reaches 120 beats per minute, therapy management engine 114 may assume the host has reached Zone 2 lactate range.

In FIG. 5B, therapy management engine 114 may begin at block 524 if the model determines the host has selected a HIIT exercise type or therapy management engine 114 has suggested HIIT based on the host's goals, time commitment, etc., at block 522. At block 524, therapy management engine 114 may determine whether the host has completed a HIIT session in the past, for which data is available, by referencing the host's profile 118. For example, therapy management engine 114 may examine exercise data in the host profile 118 to determine if there is data relating to one or more past HIIT sessions of the host.

At block 526, if the host has completed a HIIT session in the past for which data is available, therapy management engine 114 determines personalized exercise guidance for the host based on the host's own historical exercise data. For example, exercise data from the host profile 118 may be used to determine a set of exercise parameters for the host's exercise session. The set of parameters may include exercise intensity (e.g., at the outset of the session and throughout), exercise duration, etc. Exercise intensity itself can be a function of speed, resistance, incline, etc. Providing personalized guidance based on the host's own historical exercise data may be accomplished in a variety of ways. For example, the host's own historical exercise data may be used in conjunction with a rules-based model to provide the set of exercise parameters for the host's exercise session. Additionally or alternatively, one or more ML models, trained based on the host's own historical data, may be used to provide the set of exercise parameters for the host's exercise session. For example, training server system 140 may retrieve exercise data related to the specific host from the host profile 118 to train one or more ML models.

At block 528, if the host has not previously completed a HIIT session for which data is available, therapy management engine 114 may use non-personalized, population-based models to determine exercise parameters for the host, at least until the host performs one or more exercise sessions and personal exercise data is available for the host. For example, therapy management engine 114 may use a rules-based model that may define HIIT exercise parameters determined based on empirical research involving population data. For example, the HIIT exercise parameters may be determined using rules based on parameters that have been effective for a host population similar to the host to reach the HIIT intensity range. In order to determine a host population similar to the host, the population-based model may consider demographic variables (e.g., age, gender, weight, height, BMI, etc.) to determine parameters to reach the HIIT intensity range.

Additionally or alternatively, therapy management engine 114 may use one or more ML models to provide exercise parameters to the host to reach the HIIT intensity range (i.e., lactate levels in the HIIT zone). For example, an ML model may be trained based on population-based training data associated with metabolically unfit hosts who reached the HIIT intensity range, the dataset including data records each including exercise parameters, analyte data, non-analyte data, and/or other relevant information in the corresponding host's profile 118 for each exercise session. As an example, each data record in the training dataset may include time-stamped analyte data and non-analyte data for the host's exercise sessions. The data records may be labeled with one or more exercise parameters. Using such a training dataset, the model can be trained to predict one or more exercise parameters to help the host reach the HIIT intensity range.

Prior to guiding the host through an exercise session based on the exercise parameters determined at blocks 526 and 528 using personalized or population-based models, therapy management engine 114 may instruct the host to begin exercise at a warm up intensity for a duration. In certain embodiments, the intensity may be one-half of the intensity later provided to the host to reach the HIIT intensity range. In certain embodiments, the duration of the warm up intensity may be about 5 minutes or 10 minutes.

At block 530, once the warm up duration is complete, therapy management engine 114 may instruct the host to increase intensity to the suggested exercise parameters as determined in block 526 or 528.

In certain embodiments, the host may be completing the exercise session on an exercise machine (e.g., exercise machine 108), and therapy management engine 114 may automatically set or gradually increase the intensity (e.g. speed, incline, and/or resistance, etc.). Therapy management engine 114 may automatically set the exercise machine to the exercise parameters determined at block 526 and 528. In embodiments where the host is not using an exercise machine, therapy management engine 114 may instruct the host to increase the intensity of their exercise by gradually increasing various exercise parameters until the ideal HIIT intensity range is achieved. Increasing intensity may be accomplished by altering various exercise parameters associated with an exercise machine (e.g., exercise machine 108) or expending additional effort (in the form of speed or wattage) in cases where the host is not using an exercise machine.

Typically, metabolically unfit hosts may have a starting lactate level greater than 1.8 millimolar at the outset of exercise. Then, unlike a healthy host, an initial drop in lactate to reach a lactate “trough” (e.g., a negative rate of change of lactate) may be expected for metabolically unfit hosts, as the skeletal muscles begin to clear lactate from the body. However, given the high intensity nature of HIIT exercise, the lactate slope may not show an initial drop and instead begin to exponentially increase as a host begins an interval of the HIIT session. In order to ensure the host reaches the HIIT intensity range (or area under the curve) and does not overshoot the HIIT intensity range, therapy management engine 114 is configured to continuously monitor the host's lactate metrics and physiological parameters to determine optimal exercise intensity to reach the HIIT intensity range.

For example, if the host does not exercise with enough intensity, the host will not reach the optimal HIIT intensity range. Alternatively, if the host exercises with too much intensity, the host will reach the HIIT intensity range more quickly, but will then experience increased lactate levels beyond the optimal HIIT intensity range. Therefore, it is critical for therapy management engine 114 to continuously monitor the host's lactate levels, including lactate rate of change, and physiological parameters to instruct the host to increase or decrease intensity to reach the optimal HIIT intensity range, as further discussed in relation to blocks 532 and 534.

At block 532, therapy management engine 114 may determine if the lactate metrics reflect HIIT intensity range or are projected to approach the desired HIIT intensity range for the host. For example, therapy management engine 114 may monitor the values associated with area under the curve of lactate over time, a lactate delta relative to the host's baseline lactate, and/or lactate rate of increase to determine whether the host has or is projected to reach the HIIT intensity range, also referred to as defined range herein. For a metabolically unfit host, reaching 5-15 millimolar lactate, for example, corresponding to the area under the curve may be indicative of the host already reaching the desired HIIT intensity range.

In order to determine whether the host is approaching a physiological state corresponding to the HIIT intensity range, therapy management engine 114 may use one of a variety of rules-based or ML models to take the host's lactate metrics as well as other physiological parameters to project whether the host is soon approaching the physiological state corresponding to the desired HIIT intensity range.

At block 534, therapy management engine 114 may observe lactate metrics and, where the host is not trending to achieve the optimal HIIT intensity range, therapy management engine 114 may instruct the host to increase or decrease exercise intensity. For example, the host is considered not to be trending to achieve the desired lactate range when the host is either not exercising with enough intensity to achieve the HIIT intensity range, or the host is exercising with too much intensity such that the host's lactate levels will increase beyond the desired HIIT intensity range.

A variety of models (e.g., rules-based, machine learning or predictive algorithms) may be used to determine the optimal exercise parameters and, therefore, the ideal intensity to ensure that the host's lactate level reaches the desired HIIT intensity range but does not increase beyond the HIIT intensity range. In certain embodiments, a rules-based model may be utilized where various rules can be defined around a set of parameters, including the host's current lactate level, the current lactate rate of change, time since outset of exercise, intensity of exercise, as well as other parameters. For example, one example rule may dictate that if the host's current lactate level is X, the current lactate rate of change is Y and the time since the outset of exercise is Z, then the intensity should be Q. Q may be a set of exercise parameters that would bring the host into the HIIT intensity range such as speed, resistance level, elevation, wattage, etc. The rules may get much more granular and involve a whole host of other physiological and demographics metrics. For example, a rule may say if the host is above the age of 60, has a heart rate of A, temperature of B, current lactate level of X, current lactate rate of change of Y, time since the outset of exercise Z, then the intensity should be Q.

In certain other embodiments, one or more predictive algorithms or models may be used to predict the optimal intensity to achieve the desired HIIT intensity range. For example, one or more models that may be the same or different from the personalized or population based models described in relation to blocks 526 and 528 may continuously run to take a set of inputs (e.g., host's current lactate level, the current lactate rate of change, time since outset of exercise, as well as other inputs received at block 402) and output the optimal intensity and/or corresponding exercise parameters for achieving the desired HIIT intensity range.

When instructing the host to increase or decrease intensity, therapy management engine 114 may examine whether the host is exercising outdoors. For example, non-analyte sensor data from a temperature sensor may suggest that the host is exercising outdoors or the host selecting, for example, outdoor run or walk as exercise type may be used by therapy management engine 114 to determine the host is exercising outdoors. When the host is exercising outdoors, therapy management engine 114 may determine air temperature, air quality, humidity, wind speed, known allergens, and elevation when suggesting an increase or decrease intensity.

For example, based on sensor data from temperature sensors, therapy management engine 114 may notify the host that the environment is too hot and it is not safe to exercise at the usual intensity (e.g., recommend the exercise intensity as a function of temperature to reach HIIT intensity range, even if the recommended intensity is lower/higher than historical data), or that the host should stop exercising.

Further, therapy management engine 114 may instruct the host to decrease intensity or stop exercising based on the correlation between potassium levels and temperature sensor data at higher outdoor temperatures. For example, where the host may perceive that they are at the correct intensity at high temperatures, potassium levels may increase beyond expected levels due to high temperatures. Where the host perceives low to medium exertion but potassium sensors demonstrate increased potassium levels, the host may be at risk for a cardiac event (e.g., arrhythmia, cardiac arrest, etc.). Therefore, therapy management engine 114 may instruct the host to decrease intensity or stop exercising at high temperatures when the host's potassium levels increase beyond expected levels.

Further, therapy management engine 114 may use the correlation between lactate levels at specific intensity levels and temperature sensor data to provide more accurate intensity instructions to the host. For example, lactate levels may be higher in higher temperatures at the same intensity. Therefore, at high temperatures, therapy management engine 114 may instruct the host to exercise at a lower intensity to achieve the HIIT intensity range compared to the intensity required under typical temperature conditions. Conversely, at lower temperatures, the lactate response may be lower, requiring an increase in intensity to reach the optimal HIIT intensity range. Without considering temperature sensor data, at high temperatures, therapy management engine 114 may instruct the host to increase intensity which may cause the host to increase lactate beyond the optimal HIIT intensity range, and cause heat stroke or other safety concerns. At colder temperatures, therapy management engine 114 may not account for the higher intensity required to reach the HIIT intensity range when compared to typical temperatures causing a less effective exercise session.

In addition to temperature, when the host is exercising outdoors, therapy management engine 114 may also take into account elevation when providing intensity instructions. For example, if the host is jogging outdoors and is approaching an upcoming hill, therapy management engine 114 may instruct the host to maintain or even decrease the intensity. Not doing so may cause the host to exercise with too large of an increase in intensity when combined with the hill. This intensity may cause the host's lactate to overshoot the optimal HIIT intensity range. Therefore, therapy management engine 114 may take into account the change in elevation or expected change in elevation based on GPS and/or map data and adjust intensity recommendations accordingly.

Further, when the host is exercising outdoors, they may complete an exercise session (e.g., run, walk, bike, etc.) on a specific route one or more times. In such cases, therapy management engine 114 may recognize the route based on GPS data and past exercise data, and provide certain locations where the host should increase or decrease intensity in order to maintain the HIIT intensity range.

Further, whether the host is exercising indoors or outdoors, therapy management engine 114 may determine whether the host is demonstrating signs of cardiac stress based on the host's cardiac metrics (e.g., through a heart rate monitor or an ECG). For example, therapy management engine 114 may monitor various cardiac metrics for signs of atrial flutter, tachycardia, atrial fibrillation, high heart rate, etc. If the host is experiencing abnormal cardiac metrics in combination with high lactate as a result of exercise, therapy management engine 114 may instruct the host to decrease intensity and/or stop exercising and seek medical attention for potential cardiac complications.

Following a suggested increase or decrease in intensity at block 534, therapy management engine 114 may return to block 532 to determine whether the host has already reached or trending to reach the expected HIIT intensity range. If not, therapy management engine 114 returns to block 534 to adjust intensity accordingly. If yes, therapy management engine 114 will proceed to block 536.

At block 536, therapy management engine 114 may instruct the host to maintain current intensity for a specified duration (e.g., 5 minutes). The specified duration may be the ideal length of a HIIT session for the host's goals. The specified duration may also be based on the most effective length of exercise for the host based on past exercise data or the most effective length for historical hosts with similar demographics to the host.

At block 538, once the duration is complete, for non-diabetic hosts, therapy management engine 114 may guide the host to complete a cool down at a lower intensity (e.g., walking) and monitor lactate levels (or physiological parameters) to ensure that the host's lactate levels have returned to baseline level (e.g., greater than 1.8 millimolar). An active cool down allows the body to clear excess lactate as a result of exercise and optimize fat burning post-exercise.

For hosts with Type 1 and Type 2 diabetes, therapy management engine 114 host may guide the host to complete a cool down at a lower intensity (e.g., walking) and/or consume specific post-exercise nutrition to prevent immediate or possible hyperglycemia. In particular, anaerobic exercise, such as HIIT, may cause high lactate peaks during exercise and a glucose spike post-exercise when the host has cleared excess lactate from the body. If following a HIIT session, the host's glucose is low but not requiring immediate action (e.g., 70 mg/dL or 3.9 millimolar), then consuming glucose or administering insulin would not be advised as the host will experience a glucose spike once the host's liver and skeletal muscles have cleared the excess lactate during active cool down. Alternatively, if the host's glucose levels are dangerously low (e.g., 54 mg/dL), the host may be instructed to consume a small amount of glucose and monitor glucose levels post-exercise to avoid hyperglycemia. A meal or drink that is high in sugar (e.g., Gatorade) may not be recommended as it would spike lactate levels, inhibiting the cool down process, decreasing fat oxidation, and decreasing overall exercise effectiveness.

Further at block 538, therapy management engine 114 may provide feedback to the host on metabolic fitness, and effectiveness of the exercise session, as well as recommendations for future exercise sessions, diet recommendations, liver health status and estimated fat oxidation and weight loss. Feedback associated with metabolic fitness may include changes in a host's lactate response to exercise over time. For example, as the host completes exercise sessions, therapy management engine 114 may provide feedback on an improvement in lactate clearance rate over time, which demonstrates improved metabolic fitness. Feedback on improved metabolic fitness may also be based on the lactate clearance over time relative to whether the host completed a cool down following the exercise session, as a cool down encourages lactate clearance. Therapy management engine 114 may determine the intensity and duration of the cool down when monitoring the host's lactate clearance rate over time. In certain embodiments, therapy management engine 114 may instruct the host to consume a specific amount of lactate (e.g., oral lactate tolerance test) while not exercising to determine lactate clearance rate and improvement in lactate clearance rate in the absence of muscle consumption of lactate during exercise. An improvement in lactate clearance rate in response to the same amount of lactate and in the absence of exercise would indicate an improvement in the host's metabolic fitness.

In certain embodiments, feedback on the effectiveness of the exercise session may include monitoring caloric burn and overall energy expenditure following exercise. In certain embodiments, caloric burn and overall energy expenditure may be determined using the metabolic equivalent of task (MET) equation or the Harris-Benedict equation. Finally, recommendations for future exercise sessions, diet recommendations, liver health status, and estimated fat oxidation and weight loss from therapy management engine 114 are discussed in detail below.

Additionally, therapy management engine 114 may provide diet recommendations tailored to specific hosts based on the host's lactate response to HIIT. For example, diet recommendations may include specific macronutrients to consume or the suggested timing of meals relative to the exercise session. For example, following an exercise session, a host may be recommended to consume protein within a specified time period (e.g., within 20 minutes of exercise). In such an example, the host would be instructed to consume protein to build muscle mass. In another example, the host may be instructed to not consume, or limit consumption of lactate, glucose, or fructose within a specified time period (e.g., 2 hours), in order to maintain higher fat oxidation levels.

Further, therapy management engine 114 may provide feedback associated with the host's liver health. Liver health feedback may include providing the host information about a liver disease diagnosis, and/or liver disease improvement/deterioration. Information about liver disease improvement/deterioration can be obtained based on monitoring for liver disease progression using lactate measurements pre- or post-exercise (e.g., fasting lactate, overnight baseline lactate, lactate clearance rates post-exercise, lactate levels during exercise), other analyte levels (e.g., liver lipid content, liver enzyme status, glycogen storage status, bilirubin levels, or insulin resistance), and liver health testing (e.g., determining liver fat through MRI-PDFF, determining liver inflammation through ALT/AST levels, determining fibrogenic activity through Pro-C3 tests, determining blood clotting time through international normalized ration test or detecting liver elasticity through MRE imaging).

Additionally, therapy management engine 114 may project fat oxidation at rest, post-exercise and provide the host information about the projected fat oxidation. Therapy management engine 114 may consider host input (e.g., the host's diet choices, sleep, medical history, or medications taken) and past exercise data (e.g., length of exercise) to determine metabolic response to exercise and fat oxidation. Metabolic response may be the host's ability to clear lactate and glucose from the body post-exercise when compared to past exercise. Fat oxidation may be determined based on the portion of carbohydrate and lipid levels that account for the total calorie burn (e.g., using indirect calorimetry). The lipid level portion of the total calorie burn may demonstrate the host's fat oxidation as a result of exercise.

In certain embodiments, therapy management engine 114 may correlate lower lactate levels over time during subsequent exercise sessions with greater fat oxidation. Because blood lactate accumulation may be negatively correlated with fat oxidation, lower lactate accumulation as a result of exercise and/or improved lactate clearance rates would result in greater expected fat oxidation. For example, a host may experience maximal fat oxidation between 1.8 and 2.1 millimolar lactate. Therefore, if a host's baseline lactate is above 2.1 millimolar, then a reduction in lactate levels during exercise to less than 2.1 millimolar would result in increased fat oxidation for the host. Additionally, following an exercise session, a lower baseline lactate level may be indicative of improved fat oxidation for the host at rest. For example, if a host has a baseline lactate level of 3 millimolar at the outset of a first exercise session and, as a result of diet and/or exercise guidance the host's baseline lactate level improves to 2 millimolar, therapy management engine 114 may determine the host has accomplished improved fat oxidation.

Therapy management engine 114 may also measure metabolic rate of a host post-exercise and provide the host with their metabolic rate or improvement in metabolic rate relative to prior metabolic rate. Therapy management engine 114 may use peak lactate, lactate clearance rate, and lactate rate of change to predict a metabolic rate post-exercise. Therapy management engine 114 may also use indirect calorimetry, the Weir equation, or analyte measurements (e.g., glucose, free fatty acids, beta hydroxyl butyrate and glycerol) to measure a host's metabolic rate over a series of exercise sessions to determine an improvement in metabolic rate. Improvement in metabolic rate may reflect improved liver health, improving physical fitness, and weight loss over time. Therapy management engine 114 may be able to determine future weight loss if the exercise regimen is continued over a time period. For example, therapy management engine 114 may be able to project expected weight loss after one month of continued exercise.

Therapy management engine 114 may also be configured to interface with a CPAP machine which may function as an indirect calorimeter. If the host uses a CPAP machine on a daily basis, utilizing a CPAP machine as an indirect calorimeter may be beneficial to gather consistent indirect calorimetry readings. For example, the CPAP may be configured as an indirect calorimeter to measure metabolic usage of specific fuel (e.g., fat or carbohydrates) via a carbon dioxide (CO2) sensor and a flow sensor to determine the rate of CO2 production. In a CPAP machine, a CO2 sensor and a flow sensor may be added to the mask or hose of the CPAP to measure oxygen consumed from inhaled air and CO2 production from exhaled air while sleeping or at rest. The use of the CPAP machine to measure energy expenditures at night may be useful to compare with the host's metabolic rate and energy expenditures following exercise to provide as feedback to the host.

Additionally, feedback associated with metabolic fitness may be provided to the host at block 538. Metabolic fitness may be determined by the host's ability to reach the HIIT intensity range with a similar intensity in the future. For example, if the host required a certain speed or wattage to reach the HIIT intensity range in past exercise sessions, therapy management engine 114 may monitor whether this intensity is effective to reach the HIIT intensity range in the future. Over time, if the host requires greater intensity (e.g., speed or wattage) to reach the HIIT intensity range, the host may have increased metabolic fitness, as the host's liver and skeletal muscles are becoming more efficient at clearing lactate from the body at greater intensity.

In certain embodiments, feedback provided to the host at block 556 may include a reward and/or gamification feature. The host may receive various badges, stars and/or other rewards when the host progresses closer to their goal (e.g., a certain number of exercise sessions, a certain improvement in metabolic fitness, etc.). In certain embodiments, feedback provided to the host at display device 107, for example, may be configured to connect to the host's social media account where the host can share updates and rewards via social media accounts with followers and friends. Allowing the host to share with others via social media may provide support and encouragement to the host and encourage the host to continue their exercise regimen.

Therapy management engine 114 may also be configured to receive input from the host. For example, the host may be prompted to provide input to therapy management engine 114, via a user interface of display device 107, user interface 113 of exercise machine 108, etc. Therapy management engine 114 may request the host's perceived exertion level following a HIIT session or an interval during a HIIT session. In certain embodiments, the host may be given choices to select the host's perceived exertion level during the exercise session (e.g., “I feel like I can keep going at this pace” or “I am reaching my limit” or “I am feeling nauseous and cannot continue at this intensity”). Receiving input from the host regarding the perceived exertion level may assist therapy management engine 114 in suggesting an increase or decrease in intensity in future exercise sessions. For example, if the host provides input that they are nauseous and cannot continue after an exercise where the host reached the HIIT intensity range early in the exercise session and continued for a few more minutes, therapy management engine 114 may suggest the host exercise at a lower intensity in the future. The lower intensity would still allow the host to reach the HIIT intensity range and provide an effective exercise without the host feeling that they cannot continue at the intensity.

However, in some cases, a host who is taking a beta blocker medication may have a higher perceived exertion level than reality. For example, a host on a beta blocker medication may believe they are overexerting and provide input to that effect, but the host's lactate measurements may signal to therapy management engine 114 that the host achieved the ideal HIIT intensity range during the perceived overexertion. In such an example, therapy management engine 114 may be less likely to alter intensity in future exercise sessions based on the host's perceived exertion level.

In some cases, potassium metrics may be utilized to determine when the host is suffering from overexertion. For example, the host may be suffering from overexertion where the host's potassium levels reach an absolute maximum potassium level, an individualized maximum potassium level, or exceed a set duration of time where potassium levels are above the maximum potassium level. In another example, the host may also be suffering from overexertion if the host's potassium levels show a high rate of change, which can demonstrate that the host's potassium levels will eventually exceed an absolute or individualized maximum potassium level.

At block 540, once the host has completed a HIIT session, therapy management engine 114 may utilize the information obtained about the host during the exercise session (e.g., analyte data, non-analyte data, host inputs, etc.) to optimize future exercise sessions. For example, the information may be used in one or more rules-based models at block 526 during a future exercise session. In another example, the ML models utilized at blocks 526 and 528 may be personalized (e.g. trained/retrained) using the information. As such, for future exercise sessions, therapy management engine 114 may be able to more effectively predict the optimal exercise parameters that may get the specific host to the HIIT intensity range.

In FIG. 5C, therapy management engine 114 may begin at block 542 if the model determines the host has selected a resistance training exercise type or if therapy management engine 114 has suggested resistance training based on the host's goals, medical history, time commitment, etc., at block 522. At block 542, therapy management engine 114 may determine whether the host has completed a resistance training session in the past, for which data is available, by referencing the host's profile 118. For example, therapy management engine 114 my examine exercise data in the host profile 118 to determine if there is data relating to one or more past resistance training sessions of the host.

At block 544, if the host has completed a resistance training session in the past for which data is available, therapy management engine 114 determined personalized exercise guidance for the host based on the host's own historical exercise data. For example, exercise data from the host profile 118 may be used to determine a set of exercise parameters for the host's exercise session. In certain embodiments, the set of exercise parameters for resistance training may include the type of equipment to be used (e.g., free weights, barbell weights, resistance bands, etc.), type of exercises to complete (e.g., squats, bicep curls, etc.) number of repetitions, number of sets, weight amounts, time between repetitions and/or sets, tempo of repetitions, speed at which repetitions are performed, target lactate levels, etc. Providing personalized guidance based on the host's own historical exercise data may be accomplished in a variety of ways. For example, the host's own historical exercise data may be used in conjunction with a rules-based model to provide the set of exercise parameters for the host's exercise session. Additionally or alternatively, one or more ML models, trained based on the host's own historical data, may be used to provide the set of exercise parameters for the host's exercise session. For example, training server system 140 may retrieve exercise data related to the specific host from the host profile 118 to train one or more ML models.

At block 546, if the host has not previously completed a resistance training session for which data is available, therapy management engine 114 may use non-personalized, population-based models to determine exercise parameters for the host, at least until the host performs one or more resistance training sessions and personal resistance training exercise data is available for the host. For example, therapy management engine 114 may use a rules-based model that may define resistance training parameters determined based on empirical research involving population data. For example, the resistance training parameters may be determined using rules based on parameters that have been effective for a host population similar to the host to reach the resistance training intensity range. In order to determine a host population similar to the host, the population-based model may consider demographic variables (e.g., age, gender, etc.) and physiological variables (e.g., weight, height, BMI, body composition, etc.) to determine parameters to reach the resistance training intensity range.

Additionally or alternatively, therapy management engine 114 may use one or more ML models to provide exercise parameters to the host to reach the resistance training intensity range (i.e., lactate levels in the resistance training zone). For example, an ML model may be trained based on population-based training data associated with metabolically unfit hosts who reached the resistance training intensity range, the dataset including data records each including exercise parameters, analyte data, non-analyte data, and/or other relevant information in the corresponding host's profile 118 for each exercise session. As an example, each data record in the training dataset may include time-stamped analyte data and non-analyte data for the host's exercise sessions. The data records may be labeled with one or more exercise parameters. Using such a training dataset, the model can be trained to predict one or more exercise parameters to help the host reach the resistance training intensity range.

Prior to guiding the host through an exercise session based on the exercise parameters determined at blocks 544 and 546 using personalized or population-based models, therapy management engine 114 may prompt the host to provide input, via a user interface of display device 107, for example, related to any injuries the host is currently suffering from or has recently suffered from. The host may also be prompted to provide therapy management engine 114 with a risk tolerance level (e.g., the amount of risk a host is willing to assume of recurrence or worsening of an injury as a result of the resistance training session). Based on injury and risk tolerance level input, therapy management engine 114 may provide exercise parameters to prevent further injury or risk of further injury to the host based on the host's risk tolerance. In certain embodiments, therapy management engine 114 may determine ideal exercise parameters for a host based on historical host population data for hosts suffering from the same or similar injuries. Exercise parameters of historical hosts with similar reported injuries may be used to determine a predicted injury risk of specific exercise parameters (e.g., a specific exercise, muscle group, or weight) and this risk value may be compared with the host-provided risk tolerance level to provide appropriate exercise parameters to the host.

Before beginning an exercise session, therapy management engine 114 may instruct the host to begin exercise at a warm up intensity for a duration. In certain embodiments, the intensity may be one-half of the intensity later provided to the host to reach the resistance training intensity range. In certain embodiments, the duration of the warm up intensity may be about 5 minutes or 10 minutes.

At block 548, once the warm up duration is complete, therapy management engine 114 may instruct the host to increase intensity to the suggested exercise parameters as determined in block 544 or 546.

Typically, metabolically unfit hosts may have a starting lactate levels greater than 1.8 millimolar at the outset of exercise. Then, unlike a healthy host or an athlete, an initial drop in lactate to reach a lactate “trough” (e.g., a negative rate of change of lactate) may be expected for metabolically unfit hosts, as the skeletal muscles begin to clear lactate from the body. However, given the high intensity, anaerobic nature of resistance training, the lactate slope may not show an initial drop and instead lactate levels may begin to exponentially increase as a host begins the prescribed repetitions and/or sets of resistance training. In order to ensure the host reaches the resistance training intensity range (or area under the curve) and does not overshoot the resistance training intensity range, therapy management engine 114 is configured to continuously monitor the host's lactate metrics and physiological parameters to determine optimal exercise intensity and/or parameters to reach the resistance training intensity range.

Further, therapy management engine 114 may determine rest periods between sets of exercises within a resistance training session based on non-analyte sensors and/or data, such as accelerometer data. For example, a correlation of heart rate and/or respiratory rate and lactate in past exercise sessions may allow therapy management engine 114 to determine when the host is in a rest period, or instruct the host to take a rest period to remain within a desired lactate level. A determination of rest periods may allow therapy management engine 114 to predict when the host's lactate metrics may become more stable and/or decrease for a short time period. Further, a determination of rest periods may allow therapy management engine 114 to determine the hosts' real-time lactate metrics for comparison to one or more target lactate metrics. Even further, based on the duration and frequency of known rest periods in an exercise session, therapy management engine 114 may recommend varying duration and frequency of rest periods in future exercise sessions to improve future exercise efficiency.

For example, if the host's exercise parameters are not optimal (e.g., the host is not exercising with enough intensity based on the sets, repetitions, or weights), the host will not reach the optimal resistance training intensity range. Alternatively, if the host exercises with parameters that are too intense, the host will reach resistance training range more quickly, but will then experience increased lactate levels beyond the optimal resistance training intensity range. Therefore, it is critical for therapy management engine to continuously monitor the host's lactate levels and physiological parameters to instruct the host to increase or decrease intensity of parameters to reach the optimal resistance training intensity range, as further discussed in relation to blocks 550 and 552. As described herein, monitoring physiological parameters in addition to lactate levels may account for a potential lag in lactate levels as the host begins a resistance training session. Further, given resistance training often involves training a single muscle group at a time (e.g., leg muscles), lactate levels may first increase in the muscles being exercised prior to being released into the blood stream, causing further lag in systemic lactate levels. Therefore, monitoring physiological parameters may assist therapy management engine 114 in providing accurate real-time feedback to the host.

At block 550, therapy management engine 114 may determine if the host's lactate metrics reflect the resistance training intensity range or are projected to approach the desired resistance training intensity range for the host. For example, therapy management engine 114 may monitor the values associated with area under the curve of lactate over time, a lactate delta relative to the host's baseline lactate levels, and/or lactate rate of increase to determine whether the host has or is projected to reach the resistance training intensity range, also referred to as defined range herein. Additionally or alternatively, therapy management engine 114 may monitor physiological parameters to determine when the host's lactate metrics reflect the resistance training intensity range or are projected to approach the desired resistance training intensity range for the host. For a metabolically unfit host, reaching 5-15 millimolar lactate such as 6-10 millimolar lactate, for example, corresponding to the area under the curve or projected area under the curve may be indicative of the host already reaching the desired resistance training intensity range.

In order to determine whether the host is approaching a physiological state corresponding to the resistance training intensity range, therapy management engine 114 may use one of a variety of rules-based or ML models to take the host's lactate metrics as well as other physiological parameters to project whether the host is soon approaching the physiological state corresponding to the desired resistance training intensity range.

At block 552, therapy management engine 114 may observe lactate metrics and, where the host is not trending to achieve the optimal resistance training intensity range, therapy management engine 114 may instruct the host to increase or decrease exercise intensity and/or adjust exercise parameters. For example, the host is considered not to be trending to achieve the desired lactate range when the host is either not exercising with enough intensity to achieve the resistance training intensity range, or the host is increasing with too much intensity such that the host's lactate levels will increase beyond the desired resistance training intensity range.

A variety of models (e.g., rules-based, machine learning or predictive algorithms) may be used to determine the optimal exercise parameters and, therefore, the ideal intensity to ensure that the host's lactate level reaches the desired resistance training intensity range but does not increase beyond the resistance training intensity range. In certain embodiments, a rules-based model may be utilized where various rules can be defined around a set of parameters, including the host's current lactate level, the current lactate rate of change, time since outset of exercise, intensity of exercise, as well as other parameters. For example, one example rule may dictate that is the host's current lactate level is X, the current lactate rate of change is Y and the time since the outset of exercise is Z, then the intensity should be Q. Q may be a set of exercise parameters that would bring the host into the resistance training intensity range such as type of exercise, number of repetitions, number of sets, rest between sets, etc. The rules may get much more granular and involve a whole host of other physiological and demographics metrics. For example, a rule may say if the host is above the age of 60, has sarcopenia, has a heart rate of A, temperature of B, current lactate level of X, current lactate rate of change of Y, then the intensity should be Q.

In certain other embodiments, one or more predictive algorithms or models may be used to predict the optimal intensity to achieve the desired resistance training intensity range. For example, one or more models that may be the same or different from the personalized or population based models described in relation to blocks 544 and 546 may continuously run to take a set of inputs (e.g., host's current lactate level, the current lactate rate of change, as well as other inputs received at block 402) and output the optimal intensity and/or corresponding exercise parameters for achieving the desired resistance training intensity range.

In certain embodiments, as described in reference to HIIT and Zone 2, therapy management engine 114 may determine a variety of factors when instructing the host whether the increase or decrease intensity. For example, therapy management engine 114 may determine whether the host is exercising outdoors, including the temperature and altitude of the environment.

Following a suggested increase or decrease in intensity at block 552, therapy management engine 114 may return to block 548 to determine whether the host has already reached or is trending to reach the expected resistance training intensity range, If not, therapy management engine 114 returns to block 552 to adjust intensity accordingly as described above. If yes, therapy management engine 114 will proceed to block 554.

At block 554, therapy management engine 114 may instruct the host to maintain a current intensity for a specified duration (e.g., 5-10 minutes). The specified duration may be the ideal length of a resistance training session for the host's goals. The specified duration may also be based on the most effective length of exercise for the host based on past exercise data, or the most effective length for historical hosts with similar demographics to the host.

At block 556, once the duration is complete, for non-diabetic hosts, therapy management engine 114 may guide the host to complete a cool down at a lower intensity (e.g., walking) and monitor lactate levels (or physiological parameters) to ensure that the host's lactate levels have returned to baseline level (e.g., greater than 1.8 millimolar). An active cool down allows the body to clear excess lactate as a result of exercise and optimize long-term energy expenditure post-exercise.

For hosts with Type 1 and Type 2 diabetes, therapy management engine 114 may guide the host to complete a cool down at a lower intensity (e.g., walking) and/or consume specific post-exercise nutrition to prevent immediate or possible hyperglycemia. Similar to HIIT exercise, resistance training is an anaerobic exercise which may cause high lactate peaks during exercise and a glucose spike post-exercise when the host has cleared excess lactate from the body. If following a resistance training session, the host's glucose is low but not requiring immediate action (e.g., 70 mg/dL or 3.9 millimolar), then consuming glucose or administering insulin would not be advised as the host will experience a glucose spike once the host's liver and skeletal muscles have cleared the excess lactate during active cool down. Alternatively, if the host's glucose levels are dangerously low (e.g., 54 mg/dL), the host may be instructed to consume a small amount of glucose and monitor glucose levels post-exercise to avoid hyperglycemia. A meal or drink that is high in sugar (e.g., Gatorade) may not be recommended as it would spike lactate levels, inhibiting the cool down process, decreasing fat oxidation, decreasing long term energy expenditure, and decreasing overall exercise effectiveness.

Further at block 556, therapy management engine 114 may provide feedback to the host on metabolic fitness, and effectiveness of the exercise session, as well as recommendations for future exercise sessions, diet recommendations, liver health status, estimated fat oxidation, and estimated weight loss. Feedback associated with metabolic fitness may include changes in a host's lactate response to exercise over time. For example, as the host completes exercise sessions, therapy management engine 114 may provide feedback on an improvement in lactate clearance rate over time, which demonstrates improved metabolic fitness. Feedback on improved metabolic fitness may also be based on the lactate clearance over time relative to whether the host completed a cool down following the exercise session, as a cool down encourages lactate clearance. Therapy management engine 114 may determine the intensity and duration of the cool down when monitoring the host's lactate clearance rate over time. In certain embodiments, therapy management engine 114 may instruct the host to consume a specific amount of lactate (e.g., oral lactate tolerance test) while not exercising to determine lactate clearance rate and improvement in lactate clearance rate in the absence of muscle consumption of lactate during exercise. An improvement in lactate clearance rate in response to the same amount of lactate and in the absence of exercise would indicate an improvement in the host's metabolic fitness.

In certain embodiments, feedback on the effectiveness of the exercise session may include monitoring caloric burn and overall energy expenditure following exercise. In certain embodiments, caloric burn and overall energy expenditure may be determined using the metabolic equivalent of task (MET) equation or the Harris-Benedict equation. Finally, recommendations for future exercise sessions, diet recommendations, liver health status, and estimated fat oxidation and weight loss from therapy management engine 114 are discussed in detail below.

Additionally, therapy management engine 114 may provide diet recommendations tailored to specific hosts based on the host's lactate response to resistance training. For example, diet recommendations may include specific macronutrients to consume or the suggested timing of meals relative to the exercise session. For example, following an exercise session, a host may be recommended to consume protein within a specified time period (e.g., within 20 minutes of exercise). In such an example, the host would be instructed to consume protein to build muscle mass. In another example, the host may be instructed to not consume, or limit consumption of lactate, glucose, or fructose within a specified time period (e.g., 2 hours), in order to maintain higher fat oxidation levels and greater long-term energy expenditure.

Further, therapy management engine 114 may provide feedback associated with the host's liver health. Liver health feedback may include providing the host information about a liver disease diagnosis, and/or liver disease improvement or deterioration. Information about liver disease improvement or deterioration can be obtained based on monitoring for liver disease progression using lactate measurements pre- or post-exercise (e.g., fasting lactate, overnight baseline lactate, lactate clearance rates post-exercise, lactate levels during exercise), other analyte levels (e.g., liver lipid content, liver enzyme status, glycogen storage status, bilirubin levels, or insulin resistance), and liver health testing (e.g., determining liver fat through MRI-PDFF, determining liver inflammation through ALT/AST levels, determining fibrogenic activity through Pro-C3 tests, determining blood clotting time through international normalized ration test or detecting liver elasticity through MRE imaging).

Additionally, therapy management engine 114 may project fat oxidation at rest, post-exercise and provide the host information about the projected fat oxidation. Therapy management engine 114 may consider host input (e.g., the host's diet choices, sleep, medical history, or medications taken) and past exercise data (e.g., length of exercise) to determine metabolic response to exercise and fat oxidation. Metabolic response may be the host's ability to clear lactate and glucose from the body post-exercise when compared to past exercise. Fat oxidation may be determined based on the portion of carbohydrate and lipid levels that account for the total calorie burn (e.g., using indirect calorimetry). The lipid level portion of the total calorie burn may demonstrate the host's fat oxidation as a result of exercise.

In certain embodiments, therapy management engine 114 may correlate lower lactate levels over time during subsequent exercise sessions with greater fat oxidation. Because blood lactate accumulation may be negatively correlated with fat oxidation, lower lactate accumulation as a result of exercise and/or improved lactate clearance rates would result in greater expected fat oxidation.

Therapy management engine 114 may also measure metabolic rate of a host post-exercise and provide the host with their metabolic rate or improvement in metabolic rate relative to prior metabolic rate. Therapy management engine 114 may use peak lactate, lactate clearance rate, and lactate rate of change to predict a metabolic rate post-exercise. Therapy management engine 114 may also use indirect calorimetry, the Weir equation, or analyte measurements (e.g., glucose, free fatty acids, beta hydroxyl butyrate and glycerol) to measure a host's metabolic rate over a series of exercise sessions to determine an improvement in metabolic rate. Improvement in metabolic rate may reflect improved liver health, improving physical fitness, and weight loss over time. Therapy management engine 114 may be able to determine future weight loss if the exercise regimen is continued over a time period. For example, therapy management engine 114 may be able to project expected weight loss after one month of continued exercise.

Therapy management engine 114 may also be configured to interface with a CPAP machine which may function as an indirect calorimeter. If the host uses a CPAP machine on a daily basis, utilizing a CPAP machine as an indirect calorimeter may be beneficial to gather consistent indirect calorimetry readings. For example, the CPAP may be configured as an indirect calorimeter to measure metabolic usage of specific fuel (e.g., fat or carbohydrates) via a carbon dioxide (CO2) sensor and a flow sensor to determine the rate of CO2 production. In a CPAP machine, a CO2 sensor and a flow sensor may be added to the mask or hose of the CPAP to measure oxygen consumed from inhaled air and CO2 production from exhaled air while sleeping or at rest. The use of the CPAP machine to measure energy expenditures at night may be useful to compare with the host's metabolic rate and energy expenditures following exercise to provide as feedback to the host.

Additionally, feedback associated with metabolic fitness may be provided to the host at block 556. Metabolic fitness may be determined by the host's ability to reach the resistance training intensity range with a similar intensity in the future. For example, if the host required a certain speed or wattage to reach the resistance training intensity range in past exercise sessions, therapy management engine 114 may monitor whether this intensity is effective to reach the resistance training intensity range in the future. Over time, if the host requires greater intensity (e.g., higher weight, greater number of repetitions, greater number of sets, less rest time, etc.) to reach the resistance training intensity range, the host may have increased metabolic fitness, as the host's liver and skeletal muscles are becoming more efficient at clearing lactate from the body at greater intensity.

In certain embodiments, feedback provided to the host at block 538 may include a reward and/or gamification feature. The host may receive various badges, stars and/or other rewards when the host progresses closer to their goal (e.g., a certain number of exercise sessions, a certain improvement in metabolic fitness, etc.). In certain embodiments, feedback provided to the host at display device 107, for example, may be configured to connect to the host's social media account where the host can share updates and rewards via social media accounts with followers and friends. Allowing the host to share with others via social media may provide support and encouragement to the host and encourage the host to continue their exercise regimen.

Therapy management engine 114 may also be configured to receive input from the host. For example, the host may be prompted to provide input to therapy management engine 114, via a user interface of display device 107, etc. Therapy management engine 114 may request the host's perceived exertion level following a resistance training session or a specific set or exercise during a resistance training session. In certain embodiments, the host may be given choices to select the host's perceived exertion level during the exercise session (e.g., “I could not finish the recommended number of repetitions” or “I am reaching my limit”). Receiving input from the host regarding the perceived exertion level may assist therapy management engine 114 in suggesting an increase or decrease in exercise parameters and/or intensity in future exercise sessions. For example, if the host provides input that they are nauseous and cannot continue after an exercise where the host reached the resistance training intensity range early in the exercise session and continued for a few more sets, therapy management engine 114 may suggest the host complete less sets or less repetitions in the future. The lower number of repetitions or sets would still allow the host to reach the resistance training intensity range and provide an effective exercise session without the host feeling that they cannot continue or complete the exercise session.

However, in some cases, a host who is taking a beta blocker medication may have a higher perceived exertion level than reality. For example, a host on a beta blocker medication may believe they are overexerting and provide input to that effect, but the host's lactate measurements may signal to therapy management engine 114 that the host achieved the ideal resistance training intensity range during the perceived overexertion. In such an example, therapy management engine 114 may be less likely to alter intensity in future exercise sessions based on the host's perceived exertion level.

In some cases, potassium metrics may be utilized to determine when the host is suffering from overexertion. For example, the host may be suffering from overexertion where the host's potassium levels reach an absolute maximum potassium level, an individualized maximum potassium level, or exceed a set duration of time where potassium levels are above the maximum potassium level. In another example, the host may also be suffering from overexertion if the host's potassium levels show a high rate of change, which can demonstrate that the host's potassium levels will eventually exceed an absolute or individualized maximum potassium level.

At block 558, once the host has completed a resistance training session, therapy management engine 114 may utilize the information obtained about the host during the exercise session (e.g., analyte data, non-analyte data, host inputs, etc.) to optimize future exercise sessions. For example, the information may be used in one or more rules-based models at block 544 during a future exercise session. In another example, the ML models utilized at blocks 544 and 546 may be personalized (e.g. trained/retrained) using the information. As such, for future exercise sessions, therapy management engine 114 may be able to more effectively predict the optimal exercise parameters that may get the specific host to the resistance training intensity range.

### Example Methods and Systems for Optimizing Exercise Parameters to Improve the Metabolic Fitness of Metabolically Unfit Hosts Using Continuously Monitored Analyte Data

FIG. 6 is a flow diagram illustrating an example method 600 for providing therapy management support using one or more continuous analyte sensors configured to measure or otherwise indicate, at least, a host's lactate levels, and/or non-analyte sensors, in accordance with certain aspects of the present disclosure. For example, method 600 may be performed to provide therapy management support to a host using a continuous analyte monitoring system 104, as illustrated in FIGS. 1 and 2.

Method 600 may be performed by therapy management system 100 to collect data, including for example, analyte data, host information, and non-analyte sensor data mentioned above, to (1) classify whether the host is a healthy host, an athlete, or a metabolically unfit host; (2) optimize exercise for one or more of a series of exercise sessions for the host based on exercise type and host classification. In other words, the therapy management system presented herein may offer information to direct and help optimize exercise parameters, improve exercise effectiveness, and improve metabolic fitness of a metabolically unfit host.

Method 600 is described below with reference to FIGS. 1 and 2 and their components. Specifically, method 600 provides optimization of exercise by first determining the host's classification. In some cases, as described in relation to block 404 of FIG. 4, a host may not provide enough input or the therapy management engine 114 may require additional information that can be obtained from a trial exercise session to more accurately determine the host classification. The host may be metabolically unfit, a healthy host or an athlete, which may be determined from host profile 118, inputs 128, or based on the host's lactate response during a trial exercise session, for example. Note that, while method 600 is described in a particular order herein, one or more steps of method 600 may be performed concurrently and/or one or more steps of method 600 may be omitted. Specifically, as described in reference to FIG. 4, the host may provide enough input for therapy management engine 114 to classify the host without completing a trial exercise session. Therefore, blocks 602 and 604 described below may be optional and method 600 may begin at block 606.

At block 602, method 600 begins by optionally continuously monitoring a plurality of analytes of the host, such as host 102 illustrated in FIG. 1, during a trial exercise time period to obtain analyte data associated with the plurality of analytes including at least lactate. Block 602 may be performed by continuous analyte monitoring system 104 illustrated in FIGS. 1 and 2, and more specifically, continuous analyte sensor(s) 202 illustrated in FIG. 2, in certain embodiments. For example, continuous analyte monitoring system 104 may comprise a continuous lactate sensor 202 to measure the host's lactate levels.

While the main analyte for measurement described herein is lactate, in certain embodiments, other analytes may be considered. In particular, combining lactate measurements with additional analyte data may help to further inform the analysis around metabolic fitness. For example, monitoring additional types of analytes, such as glucose, insulin, glycerol, glycogen, and/or free fatty acids measured by continuous analyte monitoring system 104, may provide additional insight into the effectiveness of an exercise, and the host's metabolic fitness. Additionally, monitoring other analytes may provide supplemental information used to determine feedback for further optimizing future exercise (e.g., determining optimal exercise parameters given a host's goals and/or classification).

In certain embodiments described herein, analyte combinations, e.g., measured and collected by one (e.g., multi-analyte) or more sensors, for metabolic fitness feedback, include lactate and at least one of glucose, insulin, glycerol, glycogen, and/or free fatty acids; however, other analyte combinations may be considered for providing exercise optimization and/or metabolic fitness feedback.

For example, in certain embodiments, at block 602, continuous analyte monitoring system 104 may continuously monitor at least lactate levels of a host during a trial exercise session. In certain embodiments, the trial exercise session may be directed by or prompted by therapy management engine 114 to determine the classification of the host.

In such embodiments, the measured lactate concentrations may be used to further inform feedback on metabolic fitness and host classification.

In addition to continuously monitoring one or more analytes of a host during a first time period to obtain analyte data at block 602, optionally, in certain embodiments, at block 604, method 600 may also include monitoring other sensor data during the trial exercise time period using one or more other non-analyte sensors or devices. Block 604 may be performed by non-analyte sensors 206 and/or medical device 208 of FIG. 2, in certain embodiments.

As mentioned previously, non-analyte sensors and devices include one or more of, but are not limited to, an insulin pump, respiration rate sensor, sensors or devices provided by display device 107 or exercise machine 108 (e.g., accelerometer, camera, global positioning system (GPS), heart rate monitor, wearable blood pressure monitor, etc.) or other host accessories (e.g., a smart watch or fitness tracker), or any other sensors or devices that provide relevant information about the host (e.g., sensors on exercise equipment). Metrics, such as metrics 130 illustrated in FIG. 3, may be calculated using measured data from each of these additional sensors. Further as illustrated in FIG. 3, metrics 130 calculated from non-analyte sensor or device data may include metabolic rate, health/fitness (e.g. body temperature), heart rate, respiratory rate, etc. In certain embodiments, described in more detail below, metrics 130 calculated from non-analyte sensor or device data may be used to further inform feedback on metabolic fitness.

One or more non-analyte sensors and/or devices may be worn by a host to aid in the detection of exercise periods of the host. Non-analyte sensors such as an accelerometer and heart rate monitor, for example, may confirm the host is exercising. For example, exercise may be determined based on an elevated heart rate of the host based on heart rate monitor data, or an increase in movement based on accelerometer data. Other types of non-analyte sensor data that may be utilized to determine physical activity include accelerometer data, step rate data, exercise equipment power meter data, GPS data, heart rate data (e.g., heart rate reserve and HRV), EKG data, EMG data, respiration rate data, temperature data, blood pressure data, galvanic skin response data, oxygen uptake data, sleep data, impedance data, etc.

As the host is exercising in the trial exercise time period, the measured and collected data from analyte and non-analyte sensors, when compared to measurements during sedentary periods, may be used to analyze at least skeletal muscles and/or liver function during each of the identified periods. In particular, about 70% of lactate is cleared by the liver with contributions from the kidneys, heart and skeletal muscle during periods of sedentary activity by the host. The amount of lactate cleared by the liver may be lower than 70% during periods of physical exertion by the host (e.g., due to additional lactate being cleared the skeletal muscles and the heart). Accordingly, in certain embodiments, measured and collected data from periods of exercise and periods of sedentary activity by the host may be used to understand lactate clearance performed by the liver, kidney, heart, and/or skeletal muscles during each of these identified periods. As described in more detail below, understanding percentages of lactate clearance performed by different organs of the body may help to isolate lactate clearance performed by only the liver to better understand liver function, and any impairment where it may exist, to inform recommendations for exercise to improve liver function and provide feedback on liver disease progression and/or improvement.

At block 606, method 600 continues by classifying (e.g. discrete classification or continuous classification) the host based on analyte and non-analyte sensor data obtained from the trial exercise session. Block 606, in certain embodiments, may be performed by therapy management engine 114. As described in relation to FIG. 4 (e.g., blocks 405 and 408), host may be classified as metabolically unfit, healthy, or an athlete. In the case of a metabolically unfit host, therapy management engine 114 may identify early signs of metabolic dysfunction during the trial exercise session. Metabolic dysfunction may include impaired lactate metabolism by the liver, which results in a higher starting lactate at the outset of exercise, as well as delayed lactate clearance rate. Accordingly, high starting lactate at the outset of exercise and delayed lactate clearance rate may support a metabolically unfit classification of the host. The classification of the host may be stored in host profile 118 or historical records database 112.

At block 608, method 600 continues by optimizing an exercise session for the host based on the host's classification, as discussed in reference to FIGS. 5A-5C. For example, as described in relation to FIGS. 5A-5C, based on the host's classification and goals, therapy management engine may recommend a set of exercise parameters (e.g., type of exercise for the host (e.g., Zone 2, resistance training or HIIT), exercise intensity (e.g., speed, resistance, incline, effort level, etc.), exercise duration, etc.). At block 610, method 600 continues by monitoring the plurality of analytes continuously during the exercise session to obtain analyte data and/or monitoring other sensor data (e.g., non-analyte data) of the host. Based on the analyte and non-analyte data, therapy management engine 114 determines whether the exercise session was optimal for the host and the host's goals as discussed in relation to FIGS. 5A-5C (e.g., blocks 520, 540, and 558). Following block 610, method 600 may continue to block 612 and provide feedback to the host on metabolic fitness, and effectiveness of the exercise session, as well as recommendations for future exercise sessions, diet recommendations, liver health status, and estimated fat oxidation and weight loss, as described in relation FIGS. 5A and 5C (e.g., blocks 518, 538, and 556).

Alternatively or additionally, method 600 may proceed from block 610 to block 614. At block 614, method 600 continues by optimizing a subsequent exercise session for the host based on the monitored analyte and non-analyte data during the exercise session. Following the optimization of the exercise session at block 614, therapy management engine 114 may return to block 610 and monitor the host's analyte and non-analyte data during the subsequent exercise session to continue optimizing the host's exercise sessions based on the host's analyte and non-analyte data in response to exercise sessions over time and/or providing feedback to the host based on the exercise sessions.

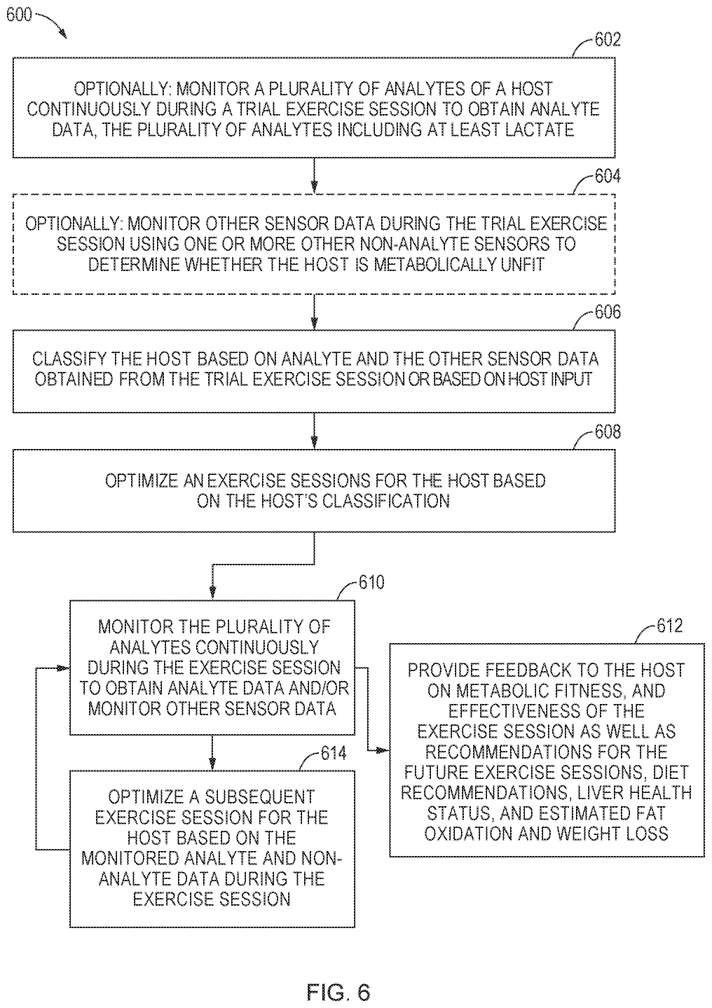

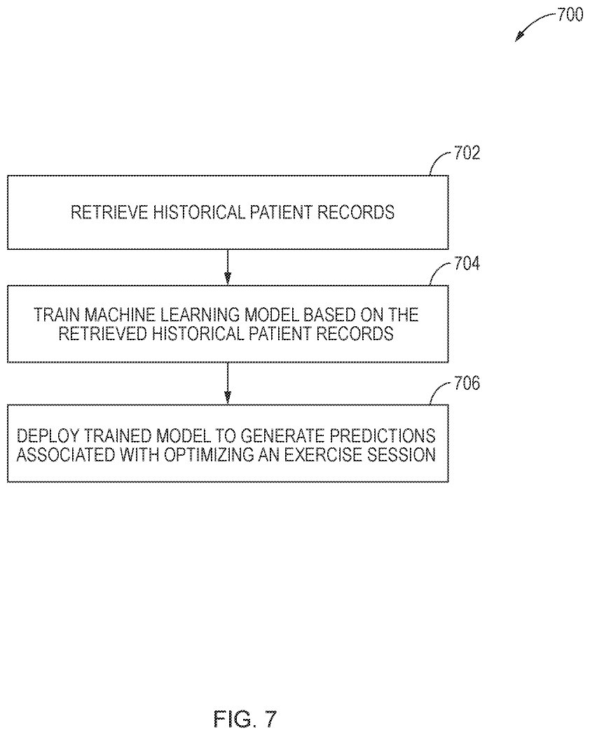

In certain embodiments, machine learning models deployed by therapy management engine 114 include one or more models trained by training server system 140, as illustrated in FIG. 1. FIG. 7 describes in further detail techniques for training the machine learning model(s) deployed by therapy management engine 114 for classifying a host, and/or optimizing an exercise session for a host, according to certain embodiments of the present disclosure.

FIG. 7 is a flow diagram depicting a method 700 for training machine learning models to classify a host, and/or optimize an exercise session for the host. In certain embodiments, the method 700 is used to train models to optimize an exercise session for a host, as illustrated in FIG. 1.

Method 700 begins, at block 702, by a training server system, such as training server system 140 illustrated in FIG. 1, retrieving data from a historical records database, such as historical records database 112 illustrated in FIG. 1. As mentioned herein, historical records database 112 may provide a repository of up-to-date information and historical information for hosts of a continuous analyte monitoring system and connected mobile health application, such as hosts of continuous analyte monitoring system 104 and application 106 or application 111 illustrated in FIG. 1, as well as data for one or more patients who are not, or were not previously, hosts of continuous analyte monitoring system 104 and/or application 106 or application 111. In certain embodiments, historical records database 112 may include one or more data sets of historical patients who are metabolically unfit, healthy hosts, and/or athletes.

Retrieval of data from historical records database 112 by training server system 140, at block 702, may include the retrieval of all, or any subset of, information maintained by historical records database 112. For example, where historical records database 112 stores information for 100,000 patients (e.g., non-hosts and hosts of continuous analyte monitoring system 104 and application 106 and/or application 111), data retrieved by training server system 140 to train one or more machine learning models may include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language may enable aggregation of healthcare historical records for baseline assessment in addition to the aggregation of de-identifiable patient data from a cloud based repository. Similarly, when integrating into the medical record databases, the integration may be accomplished by directly interfacing with the electronic medical record system or through one or more intermediary systems (e.g., an interface engine, etc.).

As an illustrative example, at block 702, training server system 140 may retrieve information for 100,000 patients with various classifications (e.g., metabolically unfit, healthy, athlete) stored in historical records database 112 to train a model to optimize one or more exercise sessions for the host and provide feedback to the host. Each of the 100,000 patients may have a corresponding data record (e.g., based on their corresponding host profile)), stored in historical records database 112. Each host profile 118 may include information, such as information discussed with respect to FIG. 3.

The training server system 140 then uses information in each of the records to train an artificial intelligence or ML model (for simplicity referred to as “ML model” herein). Examples of types of information included in a patient's host profile were provided above. The information in each of these records may be featurized (e.g., manually or by training server system 140), resulting in features that can be used as input features for training the ML model. For example, a patient record may include or be used to generate features related to the patient's demographic information (e.g., an age of a patient, a gender of the patient, etc.), analyte information, such as lactate metrics (e.g., lactate clearance rates, lactate area under the curve, an average change (e.g., average delta) in lactate clearance, other lactate metrics described herein), metabolic fitness information, non-analyte information (e.g., physiological parameters described above), and/or any other data points in the patient record (e.g., inputs 128, metrics 130, etc.). Features used to train the machine learning model(s) may vary in different embodiments.

In certain embodiments, each historical patient record retrieved from historical records database 112 is further associated with a label indicating a host classification, (e.g., a healthy host, an athlete, or a metabolically unfit host), exercise parameters, etc. What the record is labeled with would depend on what the model is being trained to predict.

At block 704, method 700 continues by training server system 140 training one or more machine learning models based on the features and labels associated with the historical patient records. In some embodiments, the training server does so by providing the features as input into a model. This model may be a new model initialized with random weights and parameters, or may be partially or fully pre-trained (e.g., based on prior training rounds). Based on the input features, the model-in-training generates some output. In certain embodiments, the output may include classification of the host, exercise parameters to optimize a host's exercise session based on the host's classification, and/or feedback to the host on metabolic fitness, effectiveness of one or more exercise sessions, recommendations for future exercise sessions, diet recommendations, liver health status, estimated fat oxidation, estimated weight loss, or similar outputs. Note that the output could be in the form of a classification, a recommendation, and/or other types of output.

In certain embodiments, training server system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to optimize a host's exercise session and provide feedback to the host more accurately.

One of a variety of machine learning algorithms may be used for training the model(s) described above. For example, one of a supervised learning algorithm, a neural network algorithm, a deep neural network algorithm, a deep learning algorithm, etc. may be used.

At block 706, training server system 140 deploys the trained model(s) to make predictions associated with optimizing an exercise session during runtime. In some embodiments, this includes transmitting some indication of the trained model(s) (e.g., a weights vector) that can be used to instantiate the model(s) on another device. For example, training server system 140 may transmit the weights of the trained model(s) to therapy management engine 114, which could execute on display device 107, exercise machine 108, etc. The model(s) can then be used to determine, in real-time, exercise parameters to optimize an exercise session of a host using application 106, and/or make other types of recommendations discussed above. In certain embodiments, the training server system 140 may continue to train the model(s) in an “online” manner by using input features and labels associated with new patient records.

Further, similar methods for training illustrated in FIG. 7 using historical patient records may also be used to train models using patient-specific records to create more personalized models for making predictions associated with host classification, and/or exercise session optimization for the host. For example, a model trained using historical patient records that is deployed for a particular host, may be further re-trained after deployment. For example, the model may be re-trained after the model is deployed for a specific patient to create a more personalized model for the patient. The more personalized model may be able to more accurately make exercise optimization suggestions for the patient based on the patient's own data (as opposed to only historical patient record data), including the patient's own inputs 128 and metrics 130.

FIG. 8 is a block diagram depicting a computing device 800 configured to execute a therapy management engine (e.g., therapy management engine 114), according to certain embodiments disclosed herein. Although depicted as a single physical device, in embodiments, computing device 800 may be implemented using virtual device(s), and/or across a number of devices, such as in a cloud environment. As illustrated, computing device 800 includes a processor 805, memory 810, storage 815, a network interface 825, and one or more I/O interfaces 820. In the illustrated embodiment, processor 805 retrieves and executes programming instructions stored in memory 810, as well as stores and retrieves application data residing in storage 815. Processor 805 is generally representative of a single CPU and/or GPU, multiple CPUs and/or GPUs, a single CPU and/or GPU having multiple processing cores, and the like. Memory 710 is generally included to be representative of a random-access memory. Storage 815 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 835 (such as keyboards, monitors, etc.) can be connected via the I/O interface(s) 820. Further, via network interface 825, computing device 800 can be communicatively coupled with one or more other devices and components, such as host database 110. In certain embodiments, computing device 800 is communicatively coupled with other devices via a network, which may include the Internet, local network(s), and the like. The network may include wired connections, wireless connections, or a combination of wired and wireless connections. As illustrated, processor 805, memory 810, storage 815, network interface(s) 825, and I/O interface(s) 820 are communicatively coupled by one or more interconnects 830. In certain embodiments, computing device 800 is representative of display device 107 associated with the host. In certain embodiments, as discussed above, the display device 107 can include the host's laptop, computer, smartphone, and the like. In another embodiment, computing device 800 is a server executing in a cloud environment. In another embodiment, computing device 800 is representative of exercise machine 108.

In the illustrated embodiment, storage 815 includes host profile 118. Memory 810 includes therapy management engine 114, which itself includes DAM 116.

FIG. 9 is a graph illustrating three lactate signal traces of three different hosts throughout three corresponding exercise sessions with increasing workload. In particular, the three lactate signal traces correspond to three different types of hosts: a metabolically unfit host having lactate signal trace 901, a healthy host having lactate signal trace 902, and an athlete host having lactate signal trace 903. Though not shown on this graph, a metabolically unfit host's lactate signal trace 901 may begin their corresponding exercise session at 0 watts of absolute workload with a starting lactate of, e.g., 3 millimolar. Upon exercising, the metabolically unfit host's lactate level may decrease to around 2 millimolar, for example. The initial decrease at the outset of exercise may be called the lactate “trough” and may only occur in metabolically unfit hosts.

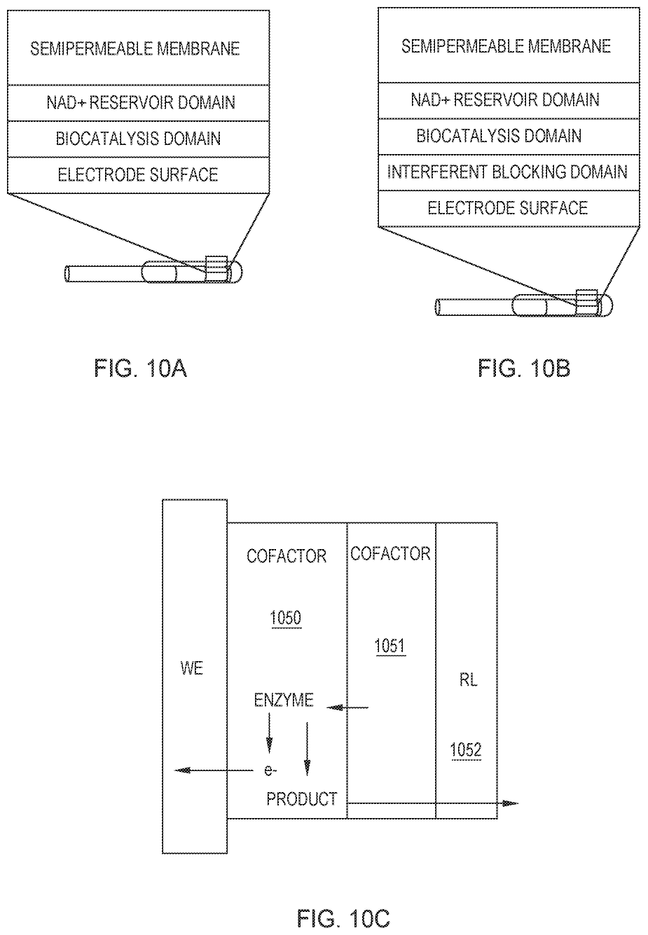

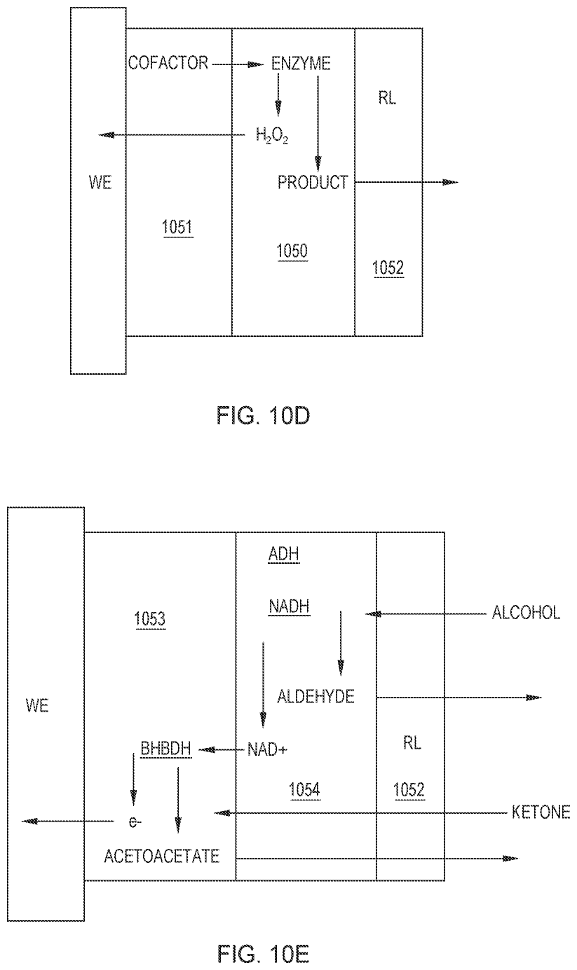

As described above, continuous analyte monitoring system 104, described in relation to FIG. 1, may be a multi-analyte sensor system including a multi-analyte sensor. FIG. 10A-14 describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl) carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro) chemical reactions involved in sensing, but forms chemical bonds with the (electro) chemically active components of the membrane. In some examples these bonds are covalent. In one example, a tether may be formed in solution prior to one or more interlayers of a membrane being formed, where the tether bonds two (electro) chemically active components directly to one another or alternately, the tether(s) bond (electro) chemically active component(s) to polymeric backbone structures. In another example, (electro) chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose: galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine: xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD (P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD (P)+) and reduced form (NAD(P)H) (H=hydrogen). The reaction of NAD+ and NADH is reversible, thus, the coenzyme can continuously cycle between the NAD (P)⁺/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD (P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD (P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD (P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 10A. With reference to FIG. 10B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 10A-10B, one or more of the membranes provides a NAD+ reservoir domain providing a reservoir for NAD+. In one example, one or more interferent blocking membranes is used, and potentiostat is utilized to measure H2O2 production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium (bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a host or health care provider.

Another example of a continuous ketone analyte detection configuration employing electrode-associated mediator-coupled diaphorase/NAD+/dehydrogenase is depicted below:

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In one example, the sensing region comprises one or more NADH: acceptor oxidoreductases and one or more NAD-dependent dehydrogenases. In one example, sensing region comprises one or more NADH: acceptor oxidoreductases and one or more NAD (P)-dependent dehydrogenases with NAD (P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 μm thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine) chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE (400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 10A-10B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. In certain embodiments, an enzyme domain 1050 comprising an enzyme (Enzyme) with an amount of cofactor (Cofactor) is positioned proximal to at least a portion of a working electrode (“WE”) surface, where the WE comprises an electrochemically reactive surface. In one example, a second membrane 1051 comprising an amount of cofactor is positioned adjacent the first enzyme domain. The amount of cofactor in the second membrane can provide an excess for the enzyme, e.g., to extend sensor life. One or more resistance domains 1052 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 10D depicts an alternative enzyme domain configuration comprising a first membrane 1051 with an amount of cofactor that is positioned more proximal to at least a portion of a WE surface. Enzyme domain 1050 comprising an amount of enzyme is positioned adjacent the first membrane.

In the membrane configurations depicted in FIGS. 10C-10D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 10E depicts another continuous multi-analyte membrane configuration, where {beta}-hydroxybutyrate dehydrogenase BHBDH in a first enzyme domain 1053 is positioned proximate to a working electrode WE and second enzyme domain 1054, for example, comprising alcohol dehydrogenase (ADH) and NADH is positioned adjacent the first enzyme domain. One or more resistance domains RL 1052 may be deployed adjacent to the second enzyme domain 1054. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with a oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD (P)+). So as to provide a continuous source of NAD(P)H or NAD (P)+, NADH oxidase or NADPH oxidases is used to oxidize the NAD(P)H or NAD (P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

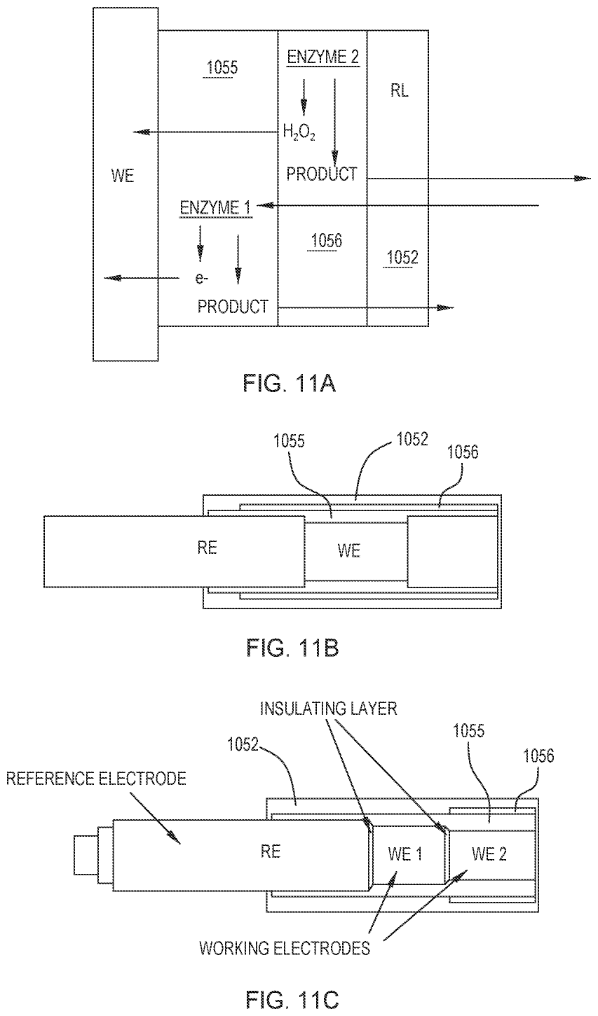

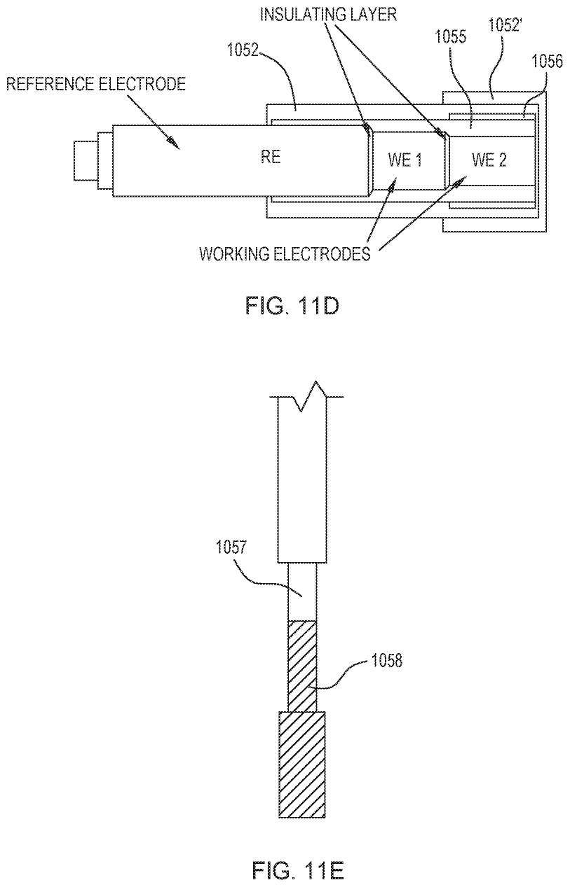

In one example, at least a dual enzyme domain configuration in which each layer contains one or more specific enzymes and optionally one or more cofactors is provided. In a broad sense, one example of a continuous multi-analyte sensor configuration is depicted in FIG. 11A where a first membrane 1055 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 1056 (EZL2) with at least one second enzyme (Enzyme 2) is positioned adjacent 1055 ELZ1, and is generally more distal from WE than EZL1. One or more resistance domains (RL) 1052 can be provided adjacent EZL2 1056, and/or between EZL1 1055 and EZL2 1056. The different enzymes catalyze the transformation of the same analyte, but at least one enzyme in EZL2 1056 provides hydrogen peroxide and the other at least one enzyme in EZL1 1055 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 11A, a first analyte diffuses through RL 1052 and into EZL2 1056 resulting in peroxide via interaction with Enzyme 2. Peroxide diffuses at least through EZL1 1055 to WE and transduces a signal that corresponds directly or indirectly to the first analyte concentration. A second analyte, which is different from the first analyte, diffuses through RL 1052 and EZL2 1056 and interacts with Enzyme 1, which results in electron transfer to WE and transduces a signal that corresponds directly or indirectly to the second analyte concentration.

As shown in FIG. 11B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 1056 providing hydrogen peroxide and the at least other enzyme in EZL1 1055 not providing hydrogen peroxide, e.g., providing electron transfer to the WE surface corresponding directly or indirectly to a concentration of the analyte.

In one example, an inner layer of the at least two enzyme domains EZL1, EZL2 1055, 1056 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 1055 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 1055 is directly adjacent the WE.

The second layer of at least dual enzyme domain (the outer layer EZL2 1056) of FIG. 11B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 1056 and through the inner layer EZL1 1055 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 1055, 1056) associated with each electrode. The two (or more) signals can be broken down into components to detect the individual signal and signal artifacts generated by each of EZL1 1055 and EZL2 1056 in response to the detection of two analytes. In some examples, each EZL detects a different analyte. In other examples, both EZLs detect the same analyte.

In another alternative exemplary configuration, as shown in FIGS. 11C-11D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate. In another example, the first analyte detected by WE1 is glucose, and the second analyte detected by WE2 is ketones.

Thus, FIGS. 11C-11D depict exemplary configurations of a continuous multi-analyte sensor construct in which EZL1 1055, EZL2 1056 and RL 1052 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 11C-11D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 11C that covers the reference electrode and WE1, WE2. An addition resistance domain is provided in the configuration of FIG. 11D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H2O2 producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 11C-11D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 11D, such an arrangement of RL's is depicted, where an additional RL 1052′ is adjacent WES2 but substantially absent from WES1.

In one example of measuring two different analytes, the above configuration comprising enzyme domain EZL1 1055 comprising one or more enzyme(s) and one or more mediators for at least one enzyme of EZL1 to provide for direct electron transfer to the WES1 and determining a concentration of at least a first analyte. In addition, enzyme domain EZL2 1056 can comprise at least one enzyme that provides peroxide (e.g., hydrogen peroxide) or consumes oxygen during catalysis with its substrate. The peroxide or the oxygen produced in EZL2 1056 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 1056. The combinations of electrode material and enzyme(s) as disclosed herein are examples and non-limiting.

In one example, the potentials of P1 and P2 can be separated by an amount of potential so that both signals (from direct electron transfer from EZL1 1055 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2 #P1 relationship, such as oxygen, and at least one enzyme-substrate combination that provide the other electrolysis compound.

For example, a continuous multi-analyte sensor configuration, for choline and glucose, in which enzyme domains EZ1 1055, EZ2 1056 were associated with different WEs, e.g., platinum WE2, and gold WE1 was prepared. In this exemplary case, EZL1 1055 contained glucose oxidase and a mediator coupled to WE1 to facilitate electron direct transfer upon catalysis of glucose, and EZL2 1056 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 11E, an example of such composite electrode surfaces is shown, in which an extended platinum covered wire 1057 is half coated with carbon 1058, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 1055) and glucose sensing (glucose oxidase in EZL2 1056). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

### Glycerol Sensor Configurations

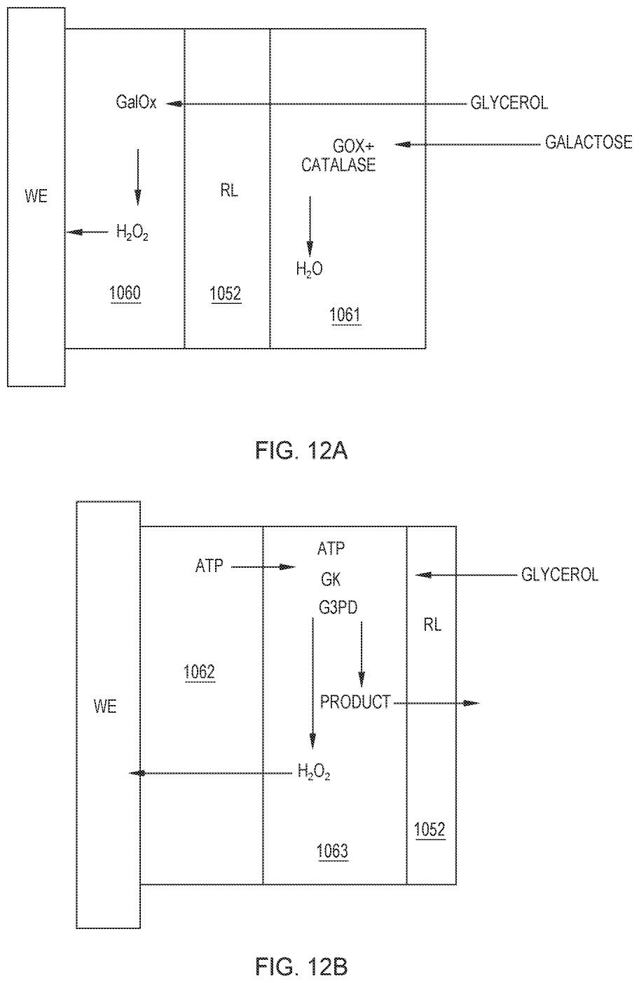

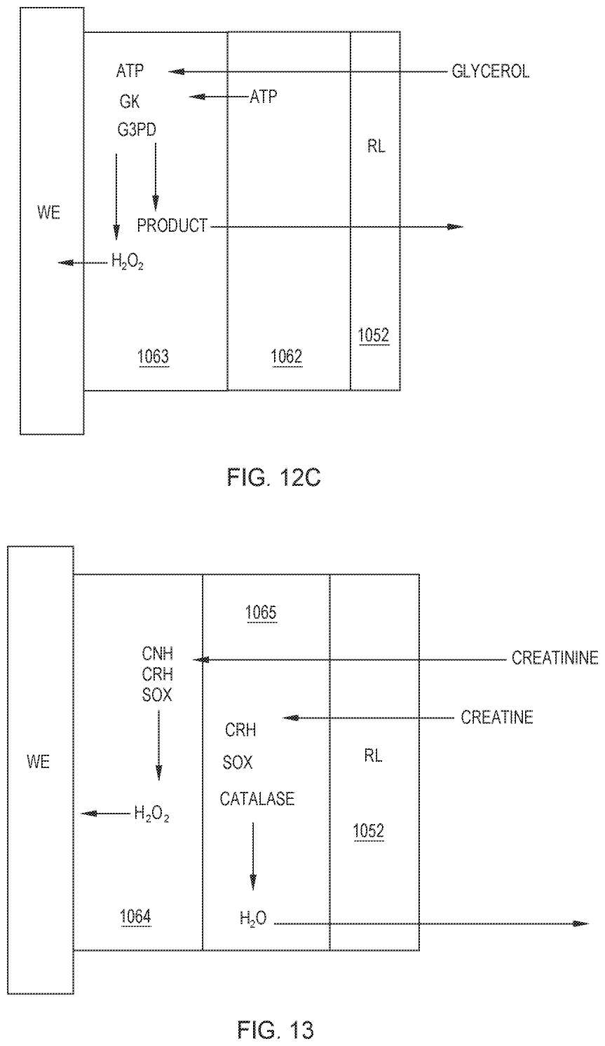

As shown in FIG. 12A, an exemplary continuous glycerol sensor configuration is depicted where a first enzyme domain EZL1 1060 comprising galactose oxidase is positioned proximal to at least a portion of a WE surface. A second enzyme domain EZL2 1061 comprising glucose oxidase and catalase is positioned more distal from the WE. As shown in FIG. 12A, one or more resistance domains (RL) 1052 are positioned between EZL1 1060 and EZL2 1061. Additional RLs can be employed, for example, adjacent to EZL2 1061.

Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/l for galactose, and ˜100 umol/l for glycerol), which compliments the aforementioned configurations.

If the GalOx present in EZL1 1060 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 1060 and EZL2 1061 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 1061 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 1052. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 12B and 12C, exemplary sensor configurations are depicted where in one example (FIG. 12B), one or more cofactors (e.g. ATP) 1062 is proximal to at least a portion of an WE surface. One or more enzyme domains 1063 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 1062. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 12C, where one or more enzyme domains 1063 comprising G3PD, at least one cofactor and at least one regenerating enzyme, are positioned proximal to at least a portion of WE surface, with one or more cofactor reservoirs 1062 adjacent to the enzyme domains comprising G3PD and more distal from the WE surface, and one or more RL's 1052 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (POX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 13 depicts an exemplary continuous sensor configuration for creatinine. In the example of FIG. 13, the sensor includes a first enzyme domain 1064 comprising CNH, CRH, and SOX are adjacent a working electrode WE, e.g., platinum. A second enzyme domain 1065 is positioned adjacent the first enzyme domain and is more distal from the WE. One or more resistance domains (RL) 1052 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

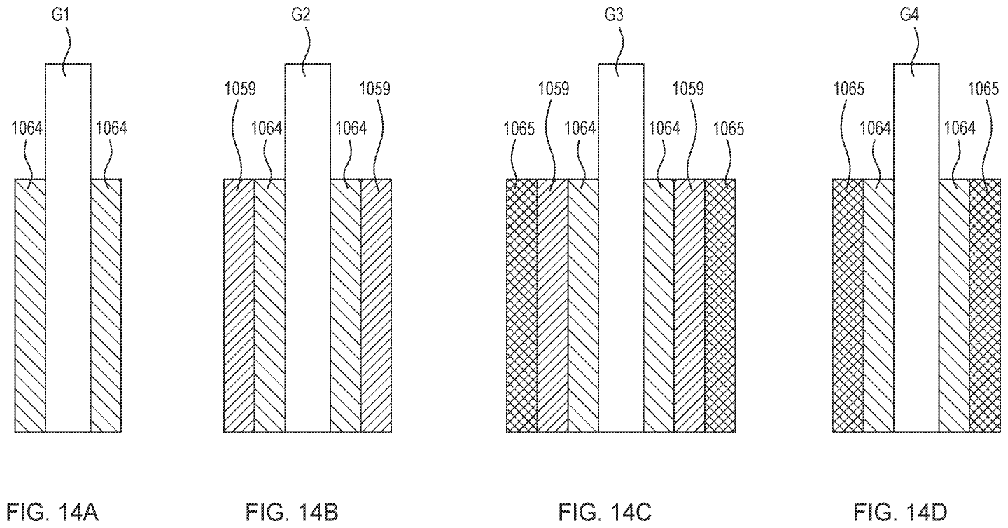

FIG. 14A-14D depict alternative continuous lactose sensor configurations. Thus, in an enzyme domain EZL1 1064 most proximal to WE (G1), comprising GalOx and lactase, provides a lactose sensor that is sensitive to galactose and lactose concentration changes and is essentially non-transducing of glucose concentration. As shown in FIGS. 12B-12D, additional layers, including non-enzyme containing layers 1059, and a lactase enzyme containing layer 1065, and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

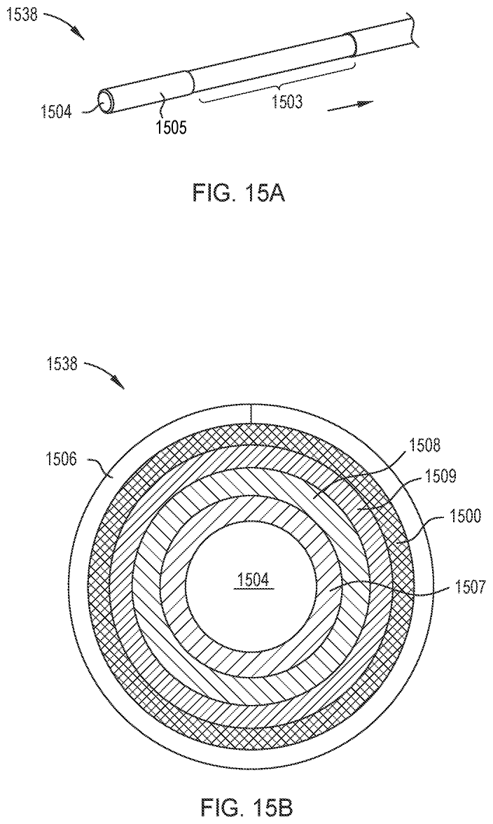

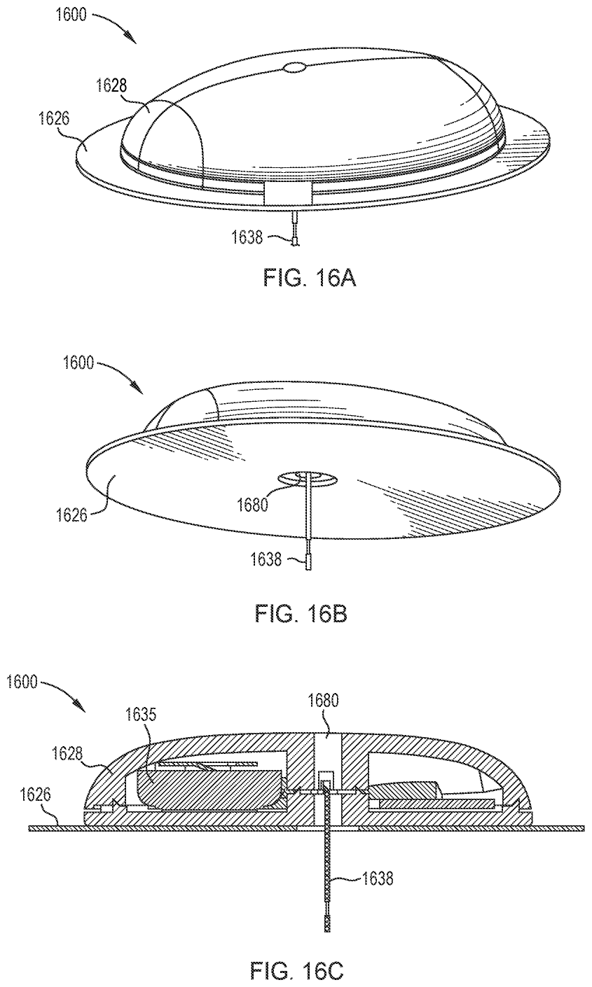

In certain embodiments, continuous analyte monitoring system 104 may be a lactate sensor, as discussed in reference to FIG. 1. FIGS. 15A-16C describe an example lactate sensor systems used to measure lactate, according to certain embodiments of the present disclosure.

FIG. 15A shows one exemplary embodiment of the physical structure of lactate sensor 1538. In this embodiment, a radial window 1503 is formed through an insulating layer 1505 to expose an electroactive working electrode of conductor material 1504. Although FIG. 15A shows a coaxial design, any form factor or shape such as a planar sheet may alternatively be used. A variety of lactate sensor designs are described in Rathee et al. “Biosensors based on electrochemical lactate detection: A comprehensive review,” Biochemistry and Biophysics Reports 5 (2016) pages 35-54, and also Rasaei et al. “Lactate Biosensors: current status and outlook” in Analytical and Bioanalytical Chemistry, September 2013, both of which are incorporated herein by reference in their entireties.

FIG. 15B is a cross-sectional view of the electroactive section of the example sensor of FIG. 15A showing the exposed electroactive surface of the working electrode surrounded by a sensing membrane in one embodiment. Such sensing membranes are present in a variety of lactate sensor designs. As shown in FIG. 15B, a sensing membrane may be deposited over at least a portion of the electroactive surfaces of the sensor (working electrode and optionally reference electrode) and provides protection of the exposed electrode surface from the biological environment, diffusion resistance of the analyte, a catalyst for enabling an enzymatic reaction, limitation or blocking of interferants, and/or hydrophilicity at the electrochemically reactive surfaces of the sensor interface.

Thus, the sensing membrane may include a plurality of domains, for example, an electrode domain 1507, an interference domain 1508, an enzyme domain 1509 (for example, including lactate oxidase), and a resistance domain 1500, and can include a high oxygen solubility domain, and/or a bioprotective domain (not shown). The membrane system can be deposited on the exposed electroactive surfaces using known thin film techniques (for example, spraying, electro-depositing, dipping, or the like). In one embodiment, one or more domains are deposited by dipping the sensor into a solution and drawing out the sensor at a speed that provides the appropriate domain thickness. However, the sensing membrane can be disposed over (or deposited on) the electroactive surfaces using any known method as will be appreciated by one skilled in the art.

The sensing membrane generally includes an enzyme domain 1509 disposed more distally situated from the electroactive surfaces than the interference domain 1508 or electrode domain 1507. In some embodiments, the enzyme domain is directly deposited onto the electroactive surfaces. In the preferred embodiments, the enzyme domain 1509 provides an enzyme such as lactose oxidase to catalyze the reaction of the analyte and its co-reactant.

The sensing membrane can also include a resistance domain 1500 disposed more distal from the electroactive surfaces than the enzyme domain 1509 because there exists a molar excess of lactate relative to the amount of oxygen in blood. However, an enzyme-based sensor employing oxygen as co-reactant is preferably supplied with oxygen in non-rate-limiting excess for the sensor to respond accurately to changes in analyte concentration rather than having the reaction unable to utilize the analyte present due to a lack of the oxygen co-reactant. This has been found to be an issue with glucose concentration monitors and is the reason why the resistance domain is included. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 2 or 3 mM. However, in a clinical setting, a linear response to glucose levels is desirable up to at least about 20 mM. To allow accurate determination of higher glucose levels, the resistance domain in the glucose monitoring context can be 200 times more permeable to oxygen than glucose. This allows an oxygen concentration high enough to make the glucose concentration the determining factor in the rate of the detected electrochemical reaction.

In some embodiments, for the lactate sensors described herein, the resistance domain can be thinner, and have a smaller difference in analyte vs. oxygen permeability, such as 50:1, or 10:1 oxygen to lactate permeability. In some embodiments, this makes the lactate sensor more sensitive to low lactate levels such as 0.5 mM or lower up to 3 or 4 mM. The resistance domain may be configured such that lactate is the rate limiting reactant at 3 mM lactate or lower, thus allowing accurate threshold detection at around 2 mM. The resistance domain may further be configured to allow oxygen to be the rate limiting reactant at lactate concentrations greater than 10 mM. These ranges may be narrowed further in some embodiments, for example the resistance domain may be configured such that lactate is the rate limiting reactant at 4 mM lactate or lower, and such that oxygen is the rate limiting reactant at lactate concentrations greater than 6 mM. In this way, the sensor itself can be optimized for early sepsis detection. It will also be appreciated that in addition to lactate, other analyte sensors can be combined with the lactate sensor described herein, such as sensors suitable for ketones, ethanol, glycerol, glucose, hormones, viruses, or any other biological component of interest.

FIGS. 16A-16C illustrate an exemplary implementation of a sensor system 104 implemented as a wearable device such as an on-skin sensor assembly 1600. As shown in FIGS. 16A-16B, on-skin sensor assembly comprises a housing 1628. An adhesive patch 1626 can couple the housing 1628 to the skin of the host. The adhesive 1626 can be a pressure sensitive adhesive (e.g., acrylic, rubber based, or other suitable type) bonded to a carrier substrate (e.g., spun lace polyester, polyurethane film, or other suitable type) for skin attachment. The housing 1628 may include a through-hole 1680 that cooperates with a sensor inserter device (not shown) that is used for implanting the sensor 1638 under the skin of a subject.

The wearable sensor assembly 1600 includes sensor electronics 1635 operable to measure and/or analyze lactate concentration indicators sensed by lactate sensor 1638. As shown in FIG. 16C, in this implementation the sensor 1638 extends from its distal end up into the through-hole 1680 and is routed to a sensor electronics 1635, typically mounted on a printed circuit board 1635 inside the housing 1628. The sensor electrodes are connected to the sensor electronics 1635. These kinds of analyte monitors are currently used in commercially available glucose monitoring systems used by diabetics, and the design principles used there can be used for an lactate monitor as well.

The housing 1628 of the sensor assembly 1600 can include a user interface for delivering messages to the patient regarding sepsis status. Because the lactate sensors described herein may, in some examples, not be a monitor that a patient will wear regularly as is the case with glucose monitors, in such examples, they may not need to include many of the features present in other monitor types such as regular wireless transmission of analyte concentration data. Accordingly, a simple user interface to just deliver warnings can be implemented. In some embodiments, the user interface could be a single light-emitting diode (LED) that is illuminated when the sensor electronics determines sepsis risk is present. Two LEDs or a two-color LED could be green when the monitor is operational and detects low risk, and red when a sepsis risk is detected and a warning is issued. The monitor may be configured to revert back to a green or low risk condition if measurements return to values appropriate for that output. To provide additional flexibility in delivering messages to patients such as error messages, time remaining to wear the device, etc., a simple dot matrix character display could be used (for example less than 200 pixels a side or a configurable 20 character LCD) that would still be inexpensive and power efficient.

### Planar Analyte Sensors

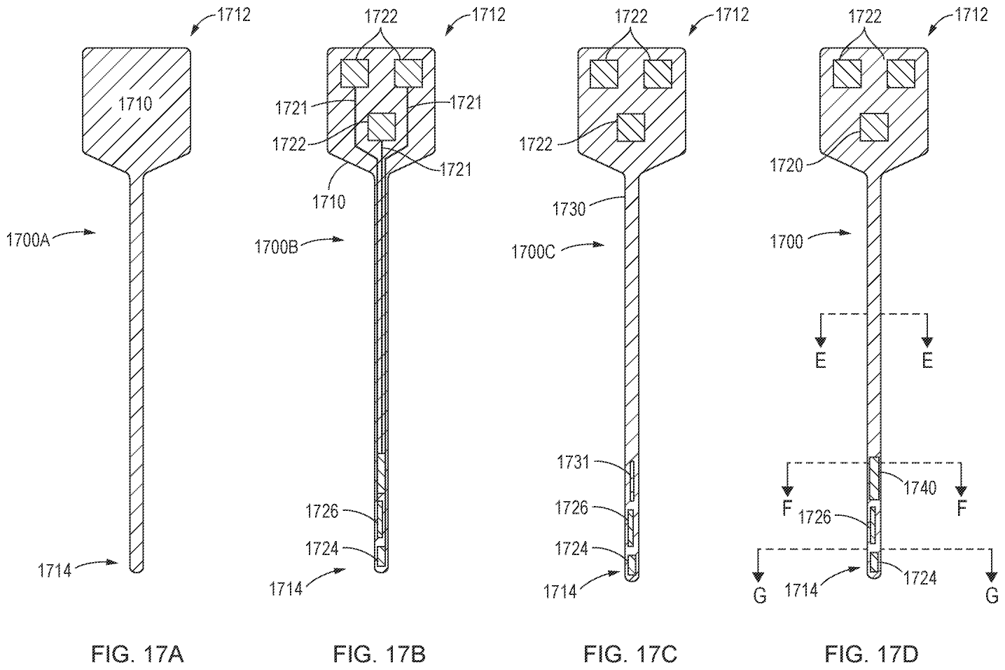

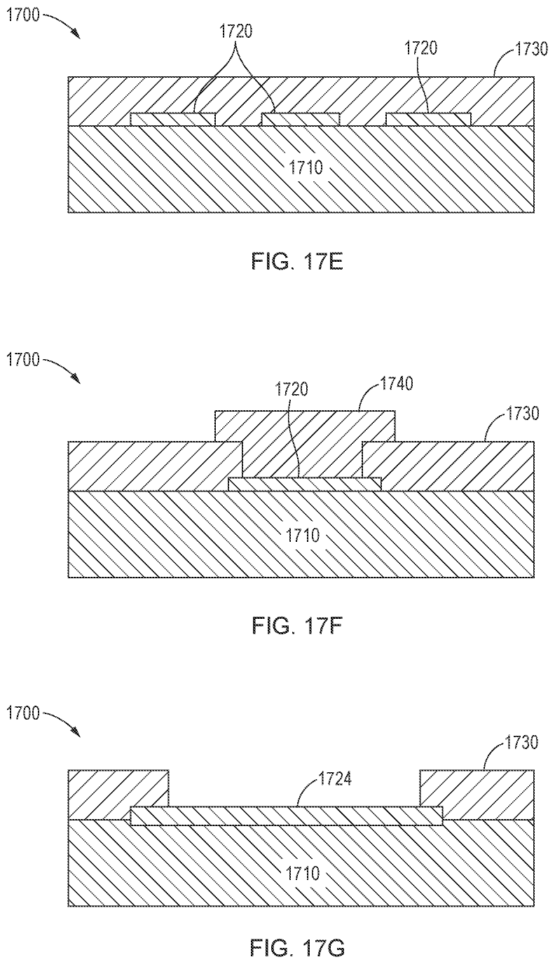

FIGS. 17A-17G depict schematic diagrams of planar analyte sensors. Each of the planar analyte sensors discussed herein can be configured to measure concentrations of one or more analytes. Planar analyte sensors can be readily manufactured and create reproducible results. Planar analyte sensors can be configured to monitor, including to continuously monitor, at least one analyte, and, in some examples, two or more analytes. The planar analyte sensors can be configured differently and may be described based on the geometry of their electrode layouts. The sensor types can include single-sided or double-sided layouts. In single-sided layouts as depicted in FIGS. 17A-17G, the electrodes can be conductive traces and can be in a co-planar arrangement, a stacked arrangement, or a staggered arrangement. In double-sided layouts, the electrodes can be in a co-planar arrangement (aligned along a shared plane in a single layer along each substrate side), a stacked arrangement (aligned along a shared plane perpendicular to the substrate side(s)), or a staggered arrangement (offset along or more plane or axis), as well as arrangements where connector pads are on a single side of the sensor, or arrangements where connector pads are on both sides of the sensor.

FIGS. 17A-17G illustrate a single sided co-planar analyte sensor assembly 1700, in accordance with an example. The sensor assembly can have a first end 1712 and a second end 1714. The sensor assembly 1700 can include substrate 1710, conductive traces 1721, connector pads 1722, working electrode 1724, counter electrode 1726, insulator 1730, and reference electrode 1740. In sensor assembly 1700, a single-sided planar configuration is used. In the sensor assembly 1700, a three-electrode sensor is shown, with a working electrode (WE) 1724, a counter electrode (CE) 1726 and a reference electrode (RE) 1740. In sensor assembly 1700, the electrodes are co-planar. In one or more implementations, an underlay configuration of single sided co-planar analyte sensor assembly 1700 or other sensor assemblies as discussed herein may enable one or more of a reference electrode (e.g., 1740) or a counter electrode (e.g., 1726) to be moved off of the sensor assembly 1700 or other sensor assembly as discussed herein to a position outside the body (e.g., as part of a wearable device such as a smartwatch)—rather than be deployed subcutaneously. This has the advantage of freeing up space in the host (e.g., in a wound pocket where the sensor assembly 1700 is inserted), where the single sided co-planar analyte sensor assembly 1700 or other sensor assemblies is deployed, for an additional working electrode and/or for detecting an additional analyte.

Moreover, by configuring a wearable device as discussed herein to include the reference (1740) and counter (1726) electrodes, such that those electrodes are externally deployed, an amount of material inserted into the body is limited. As a result, a foreign body response of a host, e.g., the response the host's immune system, may be reduced. In one or more configurations, for instance, reference electrodes (1740) may include or otherwise be formed of silver chloride (AgCl). Some hosts may have sensitivity issues with silver chloride, however. Thus, configuring the wearable device as discussed herein to include the reference electrode (1740) rather than incorporating the reference electrode (1740) as part of the in vivo portion of sensor assemblies discussed herein may reduce an immune response of such hosts, such as to reduce eye and/or skin irritation.

FIGS. 17A-17D depict top-down schematic views of the sensor assembly 1700 being produced. FIGS. 17E-17G depict cross-sectional schematic views of the sensor assembly 1700 at varying points along the length of the sensor assembly 1700.

The sensor assembly 1700 can extend between the first end 1712 and the second end 1714 and be substantially planar along its length, as measured from the first end 1712 to the second end 1714. The first end 1712 can be, for example, a connection end, such as for allowing electrical connection of the sensor assembly 1700 to a reader, computer, or other component for interpretation of signals detected with the sensor assembly 1700. The first end 1712 can host one or more connector pads 1722.

The second end 1714 can be, for example, a sensing end, for connection with or implantation in a patient, such as for detecting lactate or other analytes. The second end 1714 can host the electrodes 1724, 1726, and 1740. The second end 1714 can be the implantable portion of the sensor assembly 1700. The first end 1712 of the sensor that has the connector pads 1722 can be the proximal end of the sensor assembly 1700. The second end 1714 with the implantable portion of the sensor that contains the sensing electrodes can be the distal end of the sensor assembly 1700.

Shown in FIG. 17A, the substrate 1710 can extend between the first end 1712 and the second end 1714. The substrate 1710 can be a relatively planar material, for example, the substrate 1710 can be a thin flexible layer for hosting the other components. In some cases, the substrate 1710 can be a polymeric film, such as liquid crystal polymer (LCP), polyimide (PI), polyethylene terephthalate (PET), combinations thereof, or similar polymeric films. The substrate 1710 can have a thickness of about 25 to about 450 μm, such as a thickness of about 75 to 100 μm. In some examples, a substrate thickness of about 40 μm to about 80 μm may be used.

The conductive traces 1721, connector pads 1722, working electrode 1724, and counter electrode 1726 can be made from a conductive layer 1720 built on the substrate. The connector pads 1722 can be situated on or at the first end 1712 of the assembly 1700 and allow for electrical connection of the sensor assembly 1700. The working electrode 1724 and the counter electrode 1726 can be sensing electrodes exposed at the second end 1714 of the assembly 1700 for implantation and sensing of an analyte in a patient environment. The conductive traces 1721 can connected the electrodes 1724, 1726, to the connector pads 1722.

Shown in FIG. 17B, the conductive layer 1720 can be built up on the substrate 1710 with the conductive traces 1721, connector pads 1722, working electrode 1724, and counter electrode 1726 in a single plane or layer. The conductive layer 1720 can, for example, be made of a sputtered metal, such as titanium/gold/platinum or platinum/gold/platinum sputtered metal layers. In this case, relevant sensing surfaces such as at the working electrode 1724 can have exposed platinum for electrical connection and sensing. The reference electrode 1740 can be deposited on a base metal pad, and can be connected through additional conductive traces.

In some examples, the conductive layer 1720 is formed from a single conductor, such as gold or platinum. In other examples, the conductive layer 1720 or can be formed from more than one material, such as a thin palladium layer that is covered with gold and platinum. The composition, geometry, and exposed conductor surfaces can depend on the manufacturing method, desired mechanical properties, and requirements of the sensing chemistry. For example, the base conductive material can be formed by a less expensive material, such as silver, which is covered in strategic locations by platinum for the active sensing surfaces. In some cases, gold can be plated as the base conductor, which can be covered with platinum in order to provide both mechanical robustness and an active sensing surface for sensing hydrogen peroxide.

The conductive layer 1720, including the working electrode 1724, counter electrode 1726, connector pads 1722, and conductive traces 1721, can be formed by a variety of techniques, such as plating, sputtering, or printing. To form the structure of patterning of the conductive layers, standard photolithographic techniques, laser ablation, or printing (e.g., inkjet or screen printing) can be used.

Although certain electrode designations are shown in the supporting document, it should be understood that the size, shape, and electrode identity can be changed depending on a specific use case, such as a particular analyte to be determined. The general size and shape of the sensor is 3 mm to 4 mm wide at the proximal end (connector end) and 300-500 μm wide in the narrow implantable distal end. The overall length of the sensor is dependent on the requirements of the wearable/inserter but are generally between 15 mm and 25 mm.

Shown in FIG. 17C, the insulator 1730 can be layered on top of the conductive layer 1720 as desired. Insulating materials can be referred to as “solder mask,” “dielectric,” or “insulator.” These materials can be used to protect the conductive traces from exposure to the sample matrix and environment, as well as improve the accuracy and reliability of measurements by defining the sensing electrode area. An opening 1731 can be made for later deposition of the reference electrode 1740.

Here, the insulator 1730 can be made of an electrically insulating material deposited on top of the conductive layer to protect the conductive traces 1721 and define the openings for the connector pads 1722, and the electrodes 1724, 1726, in addition to an opening 1731 for the reference electrode 1740. The insulator 1730 can be, for example, a thin layer of solder mask.

Shown in FIG. 17D, the reference electrode 1740 material can be deposited over the designated reference electrode opening in the insulator 1730. The reference electrode 1740 material can be, for example, a silver/silver chloride formulation. It can be deposited on the designated sensing electrode pad. This reference electrode material can be deposited by a printing technique, such as screen printing, or by discrete dispense, such as a jet-valve dispenser.

FIGS. 17E-17G depict cross-section of the assembly 1700 at varying points along the body of the assembly. FIG. 17E shows a cross-section at line E-E of FIG. 17D, in a central portion of the assembly 1700. At this part of the assembly 1700, the conductive traces 1721 can be seen between the insulator 1730 and the substrate 1710. FIG. 17F shows a cross-section at line F-F of FIG. 17D, near the second end 1714 of the assembly 1700. At this part of the assembly 1700, the reference electrode 1740 can be seen on top of the conductive traces 1721. FIG. 17G depicts a cross-section at line G-G of the assembly, near the second end 1714. Here, the working electrode 1724 can be seen. The assembly 1700 is a single sided, co-planar arrangement for the electrodes 1724, 1726, 1740.

### EXAMPLE EMBODIMENTS

Implementation examples are described in the following numbered clauses:

Clause 1: A monitoring system, comprising: a continuous analyte sensor configured to generate a first set of analyte measurements associated with analyte levels of a host; and a sensor electronics module coupled to the continuous analyte sensor and configured to receive and process the first set of analyte measurements.

Clause 2: The monitoring system of Clause 1, further comprising a first receiver coupled to the sensor electronics module and configured to receive the first set of analyte measurements periodically.

Clause 3: The monitoring system of Clause 2, wherein the first receiver is further configured to process the first set of analyte measurements.

Clause 4: The monitoring system of any one of Clause 2-3, wherein the first receiver is further configured to display the first set of analyte measurements.

Clause 5: The monitoring system of any one of Clauses 1-4, wherein the continuous analyte sensor comprises: a substrate, a working electrode disposed on the substrate, a reference electrode disposed on the substrate, wherein the first set of analyte measurements generated by the continuous analyte sensor correspond to an electromotive force at least in part based on a potential difference generated between the working electrode and the reference electrode.

Clause 6: The monitoring system of any one of Clauses 1-5, wherein the continuous analyte sensor comprises a continuous lactate sensor, and the first set of analyte measurements include lactate measurements.

Clause 7: The monitoring system of Clause 6, wherein the first set of analyte measurements are received periodically.

Clause 8: The monitoring system of any one of Clauses 1-7, further comprising: one or more memories comprising executable instructions; one or more processors in data communication with the one or more memories and configured to execute the executable instructions to: classify a host as a metabolically unfit host based on the first set of analyte measurements obtained during a trial exercise session or input received from the host; and optimize an exercise session for the host based on the classification of the host.

Clause 9: The monitoring system of any one of Clauses 1-7, further comprising: one or more memories comprising executable instructions; one or more processors in data communication with the one or more memories and configured to execute the executable instructions to: determine a first fitness level of the host using the first set of analyte measurements obtained during a first period, the determination comprising: determining one or more exercise sessions for the host; determining a set of exercise parameters including intensity, exercise session type, or exercise session duration; obtaining a first set of lactate measurements indicative of a host response to the exercise during the one or more exercise sessions; and calculating, based on the first set of lactate measurements, a fitness level of the host.

Clause 10: The monitoring system of Clause 9, further comprising: receiving real time lactate measurements for the host during an exercise session; determining a target lactate level for the exercise session based on a fitness level of the host; providing feedback to the host to adjust an activity level of the host to achieve lactate levels associated with an optimized lactate measurement for the exercise session; monitoring the lactate measurements for the host after the feedback is provided to determine if the exercise session is optimized based on the adjustments; and further adjusting or maintaining the activity level to maintain a continuous lactate measurement output of the host during the exercise session.

Clause 11: The monitoring system of any one of Clauses 9-10, further comprising monitoring the host over multiple exercise sessions to determine a change in the fitness level of the host as a lactate baseline of the host or analyte measurements to a same exercise session changes.

Clause 12: The monitoring system of Clause 8, wherein the classifying the host as a metabolically unfit host based on the first set of analyte measurements comprises detecting a starting lactate level of 3 millimolar and a lactate trough in the first set of analyte measurements during the trial exercise session.

Clause 13: The monitoring system of any one of Clauses 8 or 12, wherein the classifying the host as a metabolically unfit host based on the first set of analyte measurements comprises detecting a threshold baseline lactate and a lactate trough in the first set of analyte measurements during the trial exercise session.

Clause 14: The monitoring system of any one of Clauses 8 or 12-13, wherein the classifying the host as a metabolically unfit host based on the first set of analyte measurements comprises correlating the lactate trough of the first set of analyte measurements with an exercise parameter to classify the host as the metabolically unfit host.

Clause 15: The monitoring system of any one of Clauses 8, or 12-14, wherein the classifying the host as a metabolically unfit host is further based on non-analyte data obtained from a non-analyte sensor during the trial exercise session, wherein the non-analyte data includes accelerometer data, heart rate data, heart rate variability data, oxygen saturation data, blood pressure data, or body temperature data.

Clause 16: The monitoring system of Clause 8, wherein the input received from the host include self classification information, health goals of the host, exercise goals of the host, or historical exercise data of the host.

Clause 17: The monitoring system of Clause 8, wherein optimizing the exercise session for the host comprises: determining exercise parameters for the exercise session based on the classification of the host or the first set of analyte measurements obtained during the trial exercise session; and transmitting an electronic signal to an exercise machine to cause the exercise machine to operate based on the determined exercise parameters.

Clause 18: The monitoring system of Clause 17, wherein optimizing the exercise session further comprises: monitoring a second set of analyte measurements of the host during the exercise session; determining the second set of analyte measurements is within a defined range for the exercise session; determining to maintain the exercise parameters for a specified duration of time; and causing the exercise machine to continue to operate based on the determined exercise parameters for a specified duration of time.

Clause 19: The monitoring system of any one of Clauses 8, or 17-18, wherein optimizing the exercise session further comprises: providing feedback to the host on an effectiveness of the exercise session.

Clause 20: The monitoring system of Clause 19, wherein the effectiveness of the exercise session is determined based on a caloric burn and an overall energy expenditure following the exercise session.

Clause 21: The monitoring system of any one of Clauses 18-19, wherein the one or more processors are further configured to: optimize a future exercise session based on the second set of analyte measurements and non-analyte data of the host during the exercise session.

Clause 22: The monitoring system of Clause 8, wherein optimizing the exercise session for the host comprises: determining exercise parameters for the exercise session based on the classification of the host or the first set of analyte measurements obtained during the trial exercise session; and instructing the host to exercise according to the determined exercise parameters.

Clause 23: The monitoring system of Clause 22, wherein instructing the host to exercise according to the determined exercise parameters comprises the host manually adjusting a current exercise parameter on an exercise machine to reach the determined exercise parameters.

Clause 24: The monitoring system of Clause 23, wherein exercise parameters comprise speed, incline, resistance, repetitions, or weight.

Clause 25: The monitoring system of Clause 24, wherein optimizing the exercise session further comprises: monitoring a second set of analyte measurements of the host during the exercise session; determining the second set of analyte measurements is within a defined range for the exercise session; and instructing the host to maintain the exercise parameters for a specified duration of time.

Clause 26: The monitoring system of any one of Clauses 8, or 24-25, wherein optimizing the exercise session further comprises: providing feedback to the host on an effectiveness of the exercise session.

Clause 27: The monitoring system of Clause 26, wherein the effectiveness of the exercise session is determined based on a caloric burn and an overall energy expenditure following the exercise session.

Clause 28: The monitoring system of any one of Clauses 25-26, wherein the one or more processors are further configured to: optimize a future exercise session based on the second set of analyte measurements and non-analyte data of the host during the exercise session.

Clause 29: A method for optimizing an exercise session for a host, comprising: classifying a host as a metabolically unfit host based on the first set of analyte measurements obtained during a trial exercise session or input received from the host; and optimizing an exercise session for the host based on the classification of the host.

Clause 30: A method for determining a fitness level of a host, comprising: determining a first fitness level of the host using the first set of analyte measurements obtained during a first period, the determination comprising: determining one or more exercise sessions for the host; determining a set of exercise parameters including intensity, exercise session type, or exercise session duration; obtaining a first set of lactate measurements indicative of a host response to the exercise during the one or more exercise sessions; and calculating, based on the first set of lactate measurements, a fitness level of the host.

Clause 31: The method of Clause 30, further comprising: receiving real time lactate measurements for the host during an exercise session; determining a target lactate level for the exercise session based on a fitness level of the host; providing feedback to the host to adjust an activity level of the host to achieve lactate levels associated with an optimized lactate measurement for the exercise session; monitoring the lactate measurements for the host after the feedback is provided to determine if the exercise session is optimized based on the adjustments; and further adjusting or maintaining the activity level to maintain a continuous lactate measurement output of the host during the exercise session.

Clause 32: The method of any one of Clauses 30-31, further comprising monitoring the host over multiple exercise sessions to determine a change in the fitness level of the host as a lactate baseline of the host or analyte measurements to a same exercise session changes.

Clause 33: The method of Clause 29, wherein the classifying the host as a metabolically unfit host based on the first set of analyte measurements comprises detecting a starting lactate level of 3 millimolar and a lactate trough in the first set of analyte measurements during the trial exercise session.

Clause 34: The method of any one of Clauses 29 or 33, wherein the classifying the host as a metabolically unfit host based on the first set of analyte measurements comprises detecting a threshold baseline lactate and a lactate trough in the first set of analyte measurements during the trial exercise session.

Clause 35: The method of any one of Clauses 29 or 33-34, wherein the classifying the host as a metabolically unfit host based on the first set of analyte measurements comprises correlating the lactate trough of the first set of analyte measurements with an exercise parameter to classify the host as the metabolically unfit host.

Clause 36: The method of any one of Clauses 29, or 33-35, wherein the classifying the host as a metabolically unfit host is further based on non-analyte data obtained from a non-analyte sensor during the trial exercise session, wherein the non-analyte data includes accelerometer data, heart rate data, heart rate variability data, oxygen saturation data, blood pressure data, or body temperature data.

Clause 37: The method of Clause 29, wherein the input received from the host include self classification information, health goals of the host, exercise goals of the host, or historical exercise data of the host.

Clause 38: The method of Clause 29, wherein optimizing the exercise session for the host comprises: determining exercise parameters for the exercise session based on the classification of the host or the first set of analyte measurements obtained during the trial exercise session; and transmitting an electronic signal to an exercise machine to cause the exercise machine to operate based on the determined exercise parameters.

Clause 39: The method of Clause 38, wherein optimizing the exercise session further comprises: monitoring a second set of analyte measurements of the host during the exercise session; determining the second set of analyte measurements is within a defined range for the exercise session; determining to maintain the exercise parameters for a specified duration of time; and causing the exercise machine to continue to operate based on the determined exercise parameters for a specified duration of time.

Clause 40: The method of any one of Clauses 29, or 38-39, wherein optimizing the exercise session further comprises: providing feedback to the host on an effectiveness of the exercise session.

Clause 41: The method of Clause 40, wherein the effectiveness of the exercise session is determined based on a caloric burn and an overall energy expenditure following the exercise session.

Clause 42: The method of any one of Clauses 39-40, wherein the method further comprises optimizing a future exercise session based on the second set of analyte measurements and non-analyte data of the host during the exercise session.

Clause 43: The method of Clause 42, wherein optimizing the exercise session for the host comprises: determining exercise parameters for the exercise session based on the classification of the host or the first set of analyte measurements obtained during the trial exercise session; and instructing the host to exercise according to the determined exercise parameters.

Clause 44: The method of Clause 43, wherein instructing the host to exercise according to the determined exercise parameters comprises the host manually adjusting a current exercise parameter on an exercise machine to reach the determined exercise parameters.

Clause 45: The method of Clause 44, wherein exercise parameters comprise speed, incline, resistance, repetitions, or weight.

Clause 46: The method of clause 45, wherein optimizing the exercise session further comprises: monitoring a second set of analyte measurements of the host during the exercise session; determining the second set of analyte measurements is within a defined range for the exercise session; and instructing the host to maintain the exercise parameters for a specified duration of time.

Clause 47: The method of any one of Clauses 29, or 45-46, wherein optimizing the exercise session further comprises: providing feedback to the host on an effectiveness of the exercise session.

Clause 48: The method of Clause 47, wherein the effectiveness of the exercise session is determined based on a caloric burn and an overall energy expenditure following the exercise session.

Clause 49: The method of any one of Clauses 46-47, wherein the method further comprises optimizing a future exercise session based on the second set of analyte measurements and non-analyte data of the host during the exercise session.

Clause 50: A non-transitory computer readable medium comprising instructions that when executed by one or more processors, cause the one or more processors to perform a method of optimizing an exercise session for a host, comprising: classifying a host as a metabolically unfit host based on the first set of analyte measurements obtained during a trial exercise session or input received from the host; and optimizing an exercise session for the host based on the classification of the host.

Clause 51: A non-transitory computer readable medium comprising instructions for causing a computing environment to perform a method of determining a fitness level of a host, comprising: determining a first fitness level of the host using the first set of analyte measurements obtained during a first period, the determination comprising: determining one or more exercise sessions for the host; determining a set of exercise parameters including intensity, exercise session type, or exercise session duration; obtaining a first set of lactate measurements indicative of a host response to the exercise during the one or more exercise sessions; and calculating, based on the first set of lactate measurements, a fitness level of the host.

Clause 52: The medium of Clause 52, further comprising: receiving real time lactate measurements for the host during an exercise session; determining a target lactate level for the exercise session based on a fitness level of the host; providing feedback to the host to adjust an activity level of the host to achieve lactate levels associated with an optimized lactate measurement for the exercise session; monitoring the lactate measurements for the host after the feedback is provided to determine if the exercise session is optimized based on the adjustments; and further adjusting or maintaining the activity level to maintain a continuous lactate measurement output of the host during the exercise session.

Clause 53: The medium of any one of Clauses 51-52, further comprising monitoring the host over multiple exercise sessions to determine a change in the fitness level of the host as a lactate baseline of the host or analyte measurements to a same exercise session changes.

Clause 54: The medium of Clause 50, wherein the classifying the host as a metabolically unfit host based on the first set of analyte measurements comprises detecting a starting lactate level of 3 millimolar and a lactate trough in the first set of analyte measurements during the trial exercise session.

Clause 55: The medium of any one of Clauses 50 or 54, wherein the classifying the host as a metabolically unfit host based on the first set of analyte measurements comprises detecting a threshold baseline lactate and a lactate trough in the first set of analyte measurements during the trial exercise session.

Clause 56: The medium of any one of Clauses 50 or 54-55, wherein the classifying the host as a metabolically unfit host based on the first set of analyte measurements comprises correlating the lactate trough of the first set of analyte measurements with an exercise parameter to classify the host as the metabolically unfit host.

Clause 57: The medium of any one of Clauses 50 or 54-56, wherein the classifying the host as a metabolically unfit host is further based on non-analyte data obtained from a non-analyte sensor during the trial exercise session, wherein the non-analyte data includes accelerometer data, heart rate data, heart rate variability data, oxygen saturation data, blood pressure data, or body temperature data.

Clause 58: The medium of Clause 50, wherein the input received from the host include self classification information, health goals of the host, exercise goals of the host, or historical exercise data of the host.

Clause 59: The medium of Clause 50, wherein optimizing the exercise session for the host comprises: determining exercise parameters for the exercise session based on the classification of the host or the first set of analyte measurements obtained during the trial exercise session; and transmitting an electronic signal to an exercise machine to cause the exercise machine to operate based on the determined exercise parameters.

Clause 60: The medium of Clause 59, wherein optimizing the exercise session further comprises: monitoring a second set of analyte measurements of the host during the exercise session; determining the second set of analyte measurements is within a defined range for the exercise session; determining to maintain the exercise parameters for a specified duration of time; and causing the exercise machine to continue to operate based on the determined exercise parameters for a specified duration of time.

Clause 61: The medium of any one of Clauses 50, or 59-60, wherein optimizing the exercise session further comprises: providing feedback to the host on an effectiveness of the exercise session.

Clause 62: The medium of clause 61, wherein the effectiveness of the exercise session is determined based on a caloric burn and an overall energy expenditure following the exercise session.

Clause 63: The medium of any one of Clauses 60-61, wherein the method further comprises optimizing a future exercise session based on the second set of analyte measurements and non-analyte data of the host during the exercise session.

Clause 64: The medium of Clause 63, wherein optimizing the exercise session for the host comprises: determining exercise parameters for the exercise session based on the classification of the host or the first set of analyte measurements obtained during the trial exercise session; and instructing the host to exercise according to the determined exercise parameters.

Clause 65: The medium of Clause 64, wherein instructing the host to exercise according to the determined exercise parameters comprises the host manually adjusting a current exercise parameter on an exercise machine to reach the determined exercise parameters.

Clause 66: The medium of Clause 65, wherein exercise parameters comprise speed, incline, resistance, repetitions, or weight.

Clause 67: The medium of Clause 66, wherein optimizing the exercise session further comprises: monitoring a second set of analyte measurements of the host during the exercise session; determining the second set of analyte measurements is within a defined range for the exercise session; and instructing the host to maintain the exercise parameters for a specified duration of time.

Clause 68: The medium of any one of Clauses 50 or 66-67, wherein optimizing the exercise session further comprises: providing feedback to the host on an effectiveness of the exercise session.

Clause 69: The medium of Clause 68, wherein the effectiveness of the exercise session is determined based on a caloric burn and an overall energy expenditure following the exercise session.

Clause 70: The medium of any one of Clauses 67-68, wherein the method further comprises optimizing a future exercise session based on the second set of analyte measurements and non-analyte data of the host during the exercise session.

Clause 71: A monitoring system, comprising: one or more memories comprising executable instructions; one or more processors in data communication with the one or more memories and configured to execute the executable instructions to: classify a host as a metabolically unfit host based on the first set of analyte measurements obtained during a trial exercise session or input received from the host; and optimize an exercise session for the host based on the classification of the host.

### ADDITIONAL CONSIDERATIONS

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured to generate a first set of analyte measurements associated with analyte levels of a host; and
a sensor electronics module coupled to the continuous analyte sensor and configured to receive and process the first set of analyte measurements.

2. The monitoring system of claim 1, wherein the continuous analyte sensor comprises:
a substrate,
a working electrode disposed on the substrate,
a reference electrode disposed on the substrate, wherein the first set of analyte measurements generated by the continuous analyte sensor correspond to an electromotive force at least in part based on a potential difference generated between the working electrode and the reference electrode.

3. The monitoring system of claim 1, wherein:
the continuous analyte sensor comprises a continuous lactate sensor, and
the first set of analyte measurements include lactate measurements.

4. The monitoring system of claim 3, further comprising:
one or more memories comprising executable instructions;
one or more processors in data communication with the one or more memories and configured to execute the executable instructions to:
classify a host as a metabolically unfit host based on the first set of analyte measurements obtained during a trial exercise session or input received from the host; and
optimize an exercise session for the host based on the classification of the host.

5. The monitoring system of claim 4, wherein the classifying the host as a metabolically unfit host based on the first set of analyte measurements comprises detecting a starting lactate level of 3 millimolar and a lactate trough in the first set of analyte measurements during the trial exercise session.

6. The monitoring system of claim 5, wherein the classifying the host as a metabolically unfit host based on the first set of analyte measurements comprises correlating the lactate trough of the first set of analyte measurements with an exercise parameter to classify the host as the metabolically unfit host.

7. The monitoring system of claim 4, wherein the classifying the host as a metabolically unfit host is further based on non-analyte data obtained from a non-analyte sensor during the trial exercise session, wherein the non-analyte data includes accelerometer data, heart rate data, heart rate variability data, oxygen saturation data, blood pressure data, or body temperature data.

8. The monitoring system of claim 4, wherein the input received from the host include self classification information, health goals of the host, exercise goals of the host, or historical exercise data of the host.

9. The monitoring system of claim 4, wherein optimizing the exercise session for the host comprises:
determining exercise parameters for the exercise session based on the classification of the host or the first set of analyte measurements obtained during the trial exercise session; and
transmitting an electronic signal to an exercise machine to cause the exercise machine to operate based on the determined exercise parameters.

10. The monitoring system of claim 9, wherein optimizing the exercise session further comprises:
monitoring a second set of analyte measurements of the host during the exercise session;
determining the second set of analyte measurements is within a defined range for the exercise session;
determining to maintain the exercise parameters for a specified duration of time; and
causing the exercise machine to continue to operate based on the determined exercise parameters for a specified duration of time.

11. The monitoring system of claim 4, wherein optimizing the exercise session further comprises:
providing feedback to the host on an effectiveness of the exercise session.

12. The monitoring system of claim 11, wherein the effectiveness of the exercise session is determined based on a caloric burn and an overall energy expenditure following the exercise session.

13. The monitoring system of claim 10, wherein the one or more processors are further configured to:
optimize a future exercise session based on the second set of analyte measurements and non-analyte data of the host during the exercise session.

14. The monitoring system of claim 4, wherein optimizing the exercise session for the host comprises:
determining exercise parameters for the exercise session based on the classification of the host or the first set of analyte measurements obtained during the trial exercise session; and
instructing the host to exercise according to the determined exercise parameters.

15. The monitoring system of claim 14, wherein instructing the host to exercise according to the determined exercise parameters comprises the host manually adjusting a current exercise parameter on an exercise machine to reach the determined exercise parameters.

16. The monitoring system of claim 15, wherein exercise parameters comprise speed, incline, resistance, repetitions, or weight.

17. The monitoring system of claim 14, wherein optimizing the exercise session further comprises:
monitoring a second set of analyte measurements of the host during the exercise session;
determining the second set of analyte measurements is within a defined range for the exercise session; and
instructing the host to maintain the exercise parameters for a specified duration of time.

18. The monitoring system of claim 17, wherein optimizing the exercise session further comprises:
providing feedback to the host on an effectiveness of the exercise session.

19. The monitoring system of claim 18, wherein the effectiveness of the exercise session is determined based on a caloric burn and an overall energy expenditure following the exercise session.

20. The monitoring system of claim 18, wherein the one or more processors are further configured to:
optimize a future exercise session based on the second set of analyte measurements and non-analyte data of the host during the exercise session.
